(19) United States
(12) Patent Application Publication
(10) Pub. No.: US 2025/0296932 A1
(43) Pub. Date: Sep. 25, 2025

(54) PHARMACEUTICAL COMPOSITIONS COMPRISING HIV INTEGRASE INHIBITORS

(71) Applicant: Gilead Sciences, Inc., Foster City, CA (US)

(72) Inventors: Kenneth S. Matthews, San Francisco, CA (US); Hurik Muradyan, San Mateo, CA (US); Elham Nejati, Hillsborough, CA (US)

(21) Appl. No.: 19/066,318

(22) Filed: Feb. 28, 2025

Related U.S. Application Data
(60) Provisional application No. 63/560,226, filed on Mar. 1, 2024.
(60) Provisional application No. 63/673,408, filed on Jul. 19, 2024.

(51) Int. Cl. C07D 471/12; A61K 9/00; A61K 9/20; A61K 31/551; A61P 31/18

(52) U.S. Cl. CPC C07D 471/12; A61K 9/0053; A61K 9/2018; A61K 9/2054; A61K 31/551; A61P 31/18

(57) ABSTRACT

The present disclosure relates generally to pharmaceutical compositions (e.g., solid oral dosage forms) of the compound of Formula I:

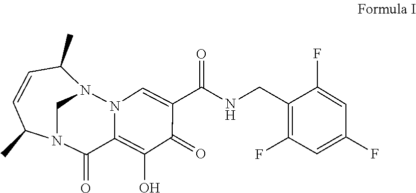

Also disclosed are methods of treating or preventing human immunodeficiency virus (HIV) infection in a human, including orally administering to the human the solid oral dosage forms or pharmaceutical compositions disclosed herein.

### CROSS-REFERENCE TO RELATED APPLICATIONS

This application claims priority to U.S. Provisional Application No. 63/560,226, filed Mar. 1, 2024, and U.S. Provisional Application No. 63/673,408, filed Jul. 19, 2024. The entire contents of these applications are hereby incorporated by reference in their entireties.

### TECHNICAL FIELD

The present invention provides pharmaceutical compositions comprising the HIV integrase inhibitor (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide, or a pharmaceutically acceptable salt thereof, and methods of treating or preventing HIV in a subject comprising orally administering to the subject a therapeutically effective amount of said pharmaceutical composition, optionally in combination with one or more other therapeutic agents.

### BACKGROUND

There is an ongoing need for antiviral agents and methods for treating HIV viral infections. There is also a constant need to develop methods for preparation and purification of the antiviral agents, as well as prepare improved pharmaceutical formulations of the same. The pharmaceutical compositions disclosed herein help meet these and other needs.

### SUMMARY

The present disclosure provides pharmaceutical compositions (e.g., solid oral dosage forms) comprising a compound of Formula I:

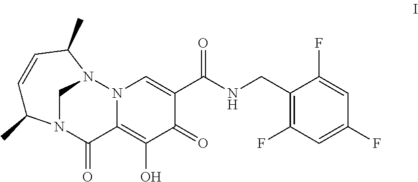

(1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide
or a pharmaceutically acceptable salt thereof, and one or more excipients.

The present disclosure also provides methods of treating or preventing HIV in a subject comprising orally administering to the subject a therapeutically effective amount of said dosage form, optionally in combination with one or more other therapeutic agents.

In an aspect, provided herein is a solid oral dosage form comprising a compound of Formula I or a pharmaceutically acceptable salt thereof, in an amount of about 5 mg to about 1400 mg, wherein the amount is based on the free acid form.

In an aspect, provided herein is a solid oral dosage form comprising a compound of Formula I or a pharmaceutically acceptable salt thereof, in an amount of about 5 mg to about 1400 mg, wherein the amount is based on the free acid form; a filler; a disintegrant; a binder; and a lubricant.

In an aspect, provided herein is a pharmaceutical composition comprising about 5 wt. % to about 35 wt. % of a compound of Formula I or a pharmaceutically acceptable salt thereof; about 50 wt. % to about 90 wt. % of a filler; about 1 wt. % to about 10 wt. % of a disintegrant; about 1 wt. % to about 10 wt. % of a binder; and about 0.1 wt. % to about 5 wt. % of a lubricant.

In an aspect, provided herein is a method of treating or preventing HIV infection in a human, comprising orally administering to the human a solid oral dosage form or a pharmaceutical composition disclosed herein.

### BRIEF DESCRIPTION OF THE DRAWINGS

FIG. 1. Shows the XRPD pattern XRPD pattern for the compound of Formula I, Form I.

FIG. 2. Shows the DSC curve for the compound of Formula I, Form I

FIG. 3. Shows the TGA curve for the compound of Formula I, Form I.

FIG. 4. Shows the DVS curve for the compound of Formula I, Form I.

FIG. 5. Shows the ball and stick drawing of the compound of Formula I, Form I.

FIG. 6. Shows the XRPD pattern for the compound of Formula I, Form II.

FIG. 7. Shows the DSC curve for the compound of Formula I, Form II.

FIG. 8. Shows the TGA curve for the compound of Formula I, Form II.

FIG. 9. Shows the XRPD pattern for the compound of Formula I, Sodium Salt, Form I.

FIG. 10. Shows the DSC curve for the compound of Formula I, Sodium Salt, Form I.

FIG. 11. Shows the TGA curve for the compound of Formula I, Sodium Salt, Form I.

FIG. 12. Shows the DVS curve for the compound of Formula I, Sodium Salt Form I.

FIG. 13. Shows the XRPD pattern for the compound of Formula I, Sodium Salt, Form II.

FIG. 14. Shows the DSC curve for the compound of Formula I, Sodium Salt, Form II.

FIG. 15. Shows the TGA curve for the compound of Formula I, Sodium Salt, Form II.

FIG. 16. Shows the XRPD pattern for the compound of Formula I, Sodium Salt, Form III.

FIG. 17. Shows the XRPD pattern for the compound of Formula I, Sodium Salt, Form IV.

FIG. 18. Shows the XRPD pattern for the compound of Formula I, Potassium Salt, Form I.

FIG. 19. Shows the DSC curve for the compound of Formula I, Potassium Salt, Form I.

FIG. 20. Shows the TGA curve for the compound of Formula I, Potassium Salt, Form I.

FIG. 21. Shows the DVS curve for the compound of Potassium Salt, Form I.

FIG. 22. Shows the atomic displacement ellipsoid drawing of the Compound of Formula I, Potassium Salt, Form I.

FIG. 23. Shows the XRPD pattern for the compound of Formula I, Potassium Salt, Form II.

FIG. 24. Shows the TGA curve for the compound of Formula I, Potassium Salt, Form II.

FIG. 25. Shows the XRPD pattern for the compound of Formula I, Potassium Salt, Form III.

FIG. 26. Shows the XRPD pattern for the compound of Formula I, Diethylamine Salt, Form I.

FIG. 27. Shows the DSC curve for the compound of Formula I, Diethylamine Salt, Form I.

FIG. 28. Shows the TGA curve for the compound of Formula I, Diethylamine Salt, Form II.

FIG. 29. Shows the DVS curve for the compound of Formula I, Diethylamine Salt, Form II.

FIG. 30. Shows the XRPD pattern for the compound of Formula I, Ammonia Salt.

FIG. 31. Shows the DSC curve for the compound of Formula I, Ammonia Salt.

FIG. 32. Shows the TGA curve for the compound of Formula I, Ammonia Salt.

FIG. 33. Shows the DVS curve for the compound of Formula I, Ammonia Salt.

FIG. 34. Shows the XRPD pattern for the compound of Formula I, Calcium Salt, Form I.

FIG. 35. Shows the DSC curve for the compound of Formula I, Calcium Salt, Form I.

FIG. 36. Shows the XRPD pattern for the compound of Formula I, Calcium Salt, Form II.

FIG. 37. Shows the XRPD pattern for the compound of Formula I, Calcium Salt, Form III.

FIG. 38. Shows the XRPD pattern for the compound of Formula I, Magnesium Salt, Form I.

FIG. 39. Shows the DSC curve for the compound of Formula I, Magnesium Salt, Form I.

FIG. 40. Shows the XRPD pattern for the compound of Formula I, Magnesium Salt, Form II.

FIG. 41. Shows the XRPD pattern for the compound of Formula I, N-Butylamine Salt.

FIG. 42. Shows the DSC curve for the compound of Formula I, N-Butylamine Salt.

FIG. 43. Shows the TGA curve for the compound of Formula I, N-Butylamine Salt.

FIG. 44. Shows the DVS curve for the compound of Formula I, N-Butylamine Salt.

FIG. 45. Shows the XRPD pattern for the compound of Formula I, diethanolamine salt.

FIG. 46. Shows the XRPD pattern for the compound of Formula I, ethylenediamine salt.

FIG. 47. Shows the XRPD pattern for the compound of Formula I, morpholine salt.

FIG. 48. Shows the XRPD pattern for the compound of Formula I, L-Arginine Salt, Form I.

FIG. 49. Shows the XRPD pattern for the compound of Formula I, L-Arginine Salt, Form II.

FIG. 50. Shows the XRPD pattern for the compound of Formula I, L-Arginine Salt, Form III.

FIG. 51. Shows the XRPD pattern for the compound of Formula I, trans-Ferulic Cocrystal, Form I.

FIG. 52. Shows the DSC curve for the compound of Formula I, trans-Ferulic Cocrystal, Form I.

FIG. 53. Shows the TGA curve for the compound of Formula I, trans-Ferulic Cocrystal, Form I.

FIG. 54. Shows the DVS curve for the compound of Formula I, trans-Ferulic Cocrystal, Form I.

FIG. 55. Shows the XRPD pattern for the compound of Formula I, trans-Ferulic Cocrystal, Form II.

FIG. 56. Shows the DSC curve for the compound of Formula I, trans-Ferulic co-crystal, Form II.

FIG. 57. Shows the TGA curve for the compound of Formula I, trans-Ferulic co-crystal, Form II.

FIG. 58. Shows the XRPD pattern for the compound of Formula I, Tromethamine Cocrystal.

FIG. 59. Shows the DSC curve for the compound of Formula I, Tromethamine Cocrystal.

FIG. 60. Shows the TGA curve for the compound of Formula I, Tromethamine Cocrystal.

FIG. 61. Shows a flow diagram illustrating exemplary preparation of the solid oral dosage forms (e.g., tablets) disclosed herein.

FIG. 62. Shows a flow diagram illustrating exemplary preparation of spray-dried dispersions (SDD) disclosed herein.

FIG. 63. Shows the potency fold change (relative to WT) for the compound of Formula I when tested for antiviral activity against HIV-1 mutants resistant to NRTI, NNRTI, PI, and CAI.

FIG. 64. Shows the potency fold change (relative to WT) for the compound of Formula I, bictegravir, and raltegravir when tested for antiviral activity against HIV-1 mutants resistant to INSTI.

### DETAILED DESCRIPTION

The present disclosure relates to pharmaceutical compositions comprising the HIV integrase inhibitor (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (Compound of Formula I, see below), which was disclosed in WO 2022/159387.

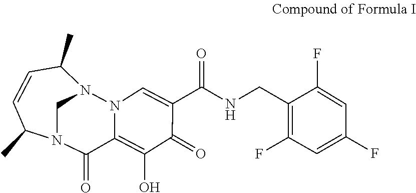

The pharmaceutical compositions disclosed herein can be useful for treating or preventing an HIV infection (e.g., HIV-1 and/or HIV-2) in a subject (e.g., a human) by administering a therapeutically effective amount of the compound of Formula I, or pharmaceutically acceptable salt thereof. In some embodiments, the compound of Formula I, or a pharmaceutically acceptable salt thereof, is administered as a monotherapy (i.e., in the absence of an additional therapeutic agent). In some embodiments, the compound of Formula I, or a pharmaceutically acceptable salt thereof, is administered in combination with one or more other therapeutic agents, such as anti-HIV agents. In some embodiments, the compound of Formula I, or a pharmaceutically acceptable salt thereof, is administered orally.

### 1. Definitions

Unless the context requires otherwise, throughout the present description and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment described herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “crystalline form” is meant to refer to a certain lattice configuration of a crystalline substance (e.g., a salt or a cocrystal). Different crystalline forms of the same substance typically have different crystalline lattices (e.g., unit cells) which are attributed to different physical properties that are characteristic of each of the crystalline forms. In some instances, different lattice configurations have different water or solvent content giving rise to solvated or hydrated crystalline forms. The term “solvated,” as used herein, is meant to refer to a crystalline form that includes solvent molecules in the crystalline lattice. The term “hydrated,” as used herein, is meant to refer to a crystalline form that is solvated, where the solvent is water and water molecules are included in the crystalline lattice. Example “hydrated” crystalline forms include hemihydrates, monohydrates, dihydrates, and the like. Other hydrated forms such as channel hydrates and the like are also included within the meaning of the term. The term “fully hydrated” is meant to refer to where the water content of the hydrate is present in the expected stoichiometric amounts. The term “partially hydrated” is meant to refer to where the water content of the hydrate is present in less than the expected stoichiometric amounts (e.g., where some of the water of a monohydrate has been removed). Similarly, the term “unsolvated” or “anhydrous” refers to a crystalline form being substantially free of solvent or water, respectively, although some residual solvent or water may be present, for example, left over from the processes used to prepare the crystalline form.

The different crystalline forms can be identified by solid state characterization methods such as by X-ray powder diffraction (XRPD). Other characterization methods such as differential scanning calorimetry (DSC), thermogravimetric analysis (TGA), and dynamic vapor sorption (DVS) further help identify the form as well as help determine stability and solvent/water content.

An XRPD pattern of reflections (peaks) is typically considered a fingerprint of a particular crystalline form. Unless otherwise stated, XRPD patterns referred to herein were conducted on a diffractometer (PANanalytical XPERT-PRO, PANanalytical B.V., Almelo, Netherlands) using copper radiation (Cu Kα, λ=1.5418 Å). Samples were prepared for analysis by depositing the powdered sample in the center of an aluminum holder equipped with a zero background plate. The generator was operated at a voltage of 45 kV and amperage of 40 mA. Slits used were Soller 0.02 rad., antiscatter 1.0°, and divergence. The sample rotation speed was 2 sec. Scans were performed from 2 to 40° 20 during 15 min with a step size of 0.0167° 2θ. Data analysis was performed by X'Pert Highscore version 2.2c (PANalytical B.V., Almelo, Netherlands) and X'Pert data viewer version 1.2d (PANalytical B.V., Almelo, Netherlands).

It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The d₉₀ values referred herein describe the size where ninety percent of particles in a sample have a smaller particle size than the specified d₉₀ value. For example, a d₉₀ of about 4 μm, means that 90% of the particles in the sample are smaller than 4 μm.

“Excipient” includes without limitation any pharmaceutically acceptable adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, basifying agent, solubilizer, glidant, filler, binder, lubricant, disintegrant, pH modifier, or coating agent, which has been approved by the United States Food and Drug Administration as being acceptable for use in humans or domestic animals. In some embodiments, such components are present in admixture within a solid oral dosage form (e.g., tablets).

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66 (1), 1-19 (1977), and Remington: The Science and Practice of Pharmacy, R. Hendrickson, ed., 21st edition, Lippincott, Williams & Wilkins, Philadelphia, PA, (2005), at p. 732, Table 38-5, both of which are hereby incorporated by reference herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

As used herein, the terms “prevention” or “preventing” refers to the administration of a compound, composition, or pharmaceutically salt according to the present disclosure pre- or post-exposure of the human to the virus but before the appearance of symptoms of the disease, and/or prior to the detection of the virus in the blood. The terms also refer to prevention of the appearance of symptoms of the disease and/or to prevent the virus from reaching detectible levels in the blood. The term includes both pre-exposure prophylaxis (PrEP), as well as post-exposure prophylaxis (PEP) and event driven or “on demand” prophylaxis. The term also refers to prevention of perinatal transmission of HIV from mother to baby, by administration to the mother before giving birth and to the child within the first days of life. The term also refers to prevention of transmission of HIV through blood transfusion.

“Subject” refers to an animal, such as a mammal (e.g., a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a composition or a compound or pharmaceutically acceptable salts, isomer, or a mixture thereof, described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition responsive to HIV activity. The therapeutically effective amount may vary depending on the subject, and the disease or condition being treated, the weight and age of the subject, the severity of the disease or condition, and the manner of administering, which can readily be determined by one of ordinary skill in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”.

The invention herein is also meant to encompass all pharmaceutically acceptable salts and/or co-crystals of the compound of Formula I being isotopically-labeled by having one or more atoms replaced by an atom having a different atomic mass or mass number. Examples of isotopes that can be incorporated into the described compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled salts and/or co-crystals of tenofovir alafenamide, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts and/or co-crystals of the compound of Formula I can generally be prepared by conventional techniques known to those skilled in the art.

### II. Compound of Formula I

The methods and pharmaceutical compositions described herein utilize the compound of Formula I, wherein the compound of Formula I is in the form of a free acid and/or a pharmaceutically acceptable salt thereof. In some embodiments, the pharmaceutical composition comprises the compound of Formula I. In some embodiments, the pharmaceutical composition comprises a pharmaceutically acceptable salt of the compound of Formula I. The compound of Formula I, or pharmaceutically acceptable salt thereof, can be crystalline, amorphous, or a combination thereof. In some embodiments, the compound of Formula I, or pharmaceutically acceptable salt thereof, is crystalline. In some embodiments, the compound of Formula I, or pharmaceutically acceptable salt thereof, is amorphous.

### Formula I, Form I

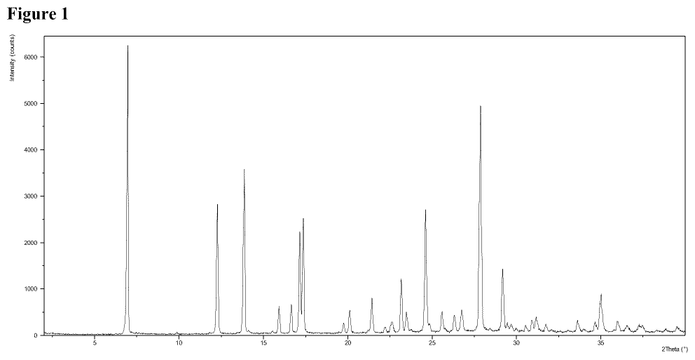

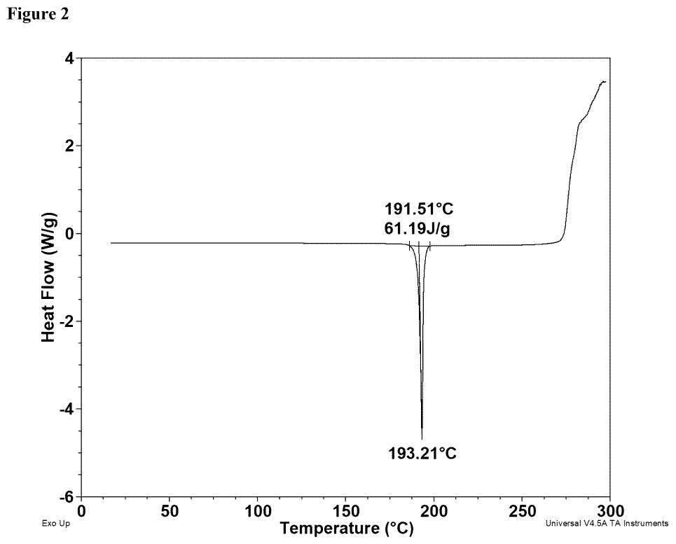

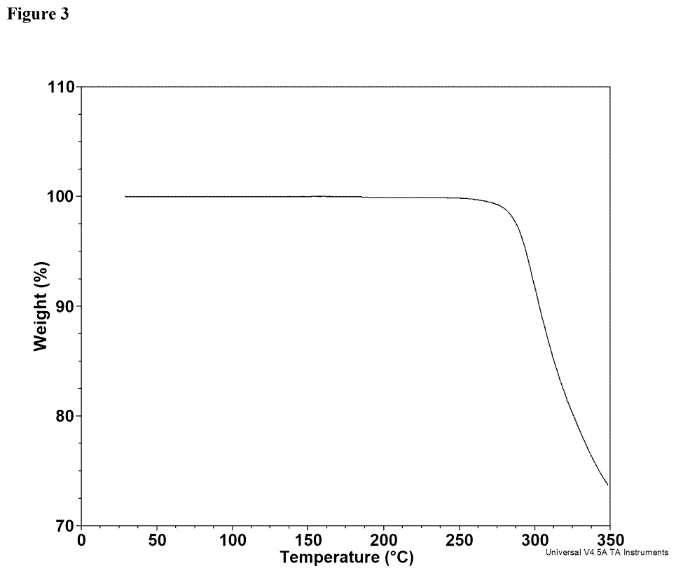

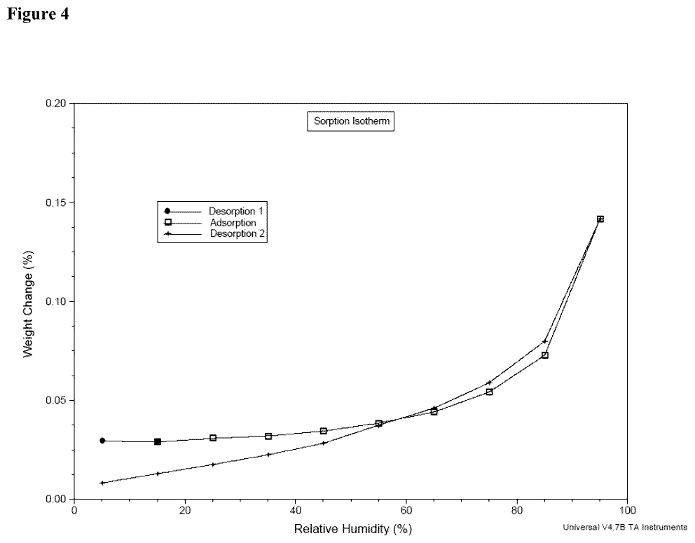

In some embodiments, the compound of Formula I is crystalline. In some embodiments, the compound of Formula I is crystalline Form I (Formula I, Form I), wherein the crystal structure exhibits an X-ray powder diffraction (XRPD) pattern substantially as shown in FIG. 1. Formula I, Form I may exhibit a differential scanning calorimetry (DSC) thermogram substantially as shown in FIG. 2. Formula I, Form I may exhibit a thermogravimetric analysis (TGA) graph substantially as shown in FIG. 3. Formula I, Form I may exhibit a dynamic vapor sorption (DVS) curve substantially as shown in FIG. 4.

The term “substantially as shown in” when referring, for example, to an XRPD pattern, a DSC thermogram, or a TGA graph includes a pattern, thermogram or graph that is not necessarily identical to those depicted herein, but that falls within the limits of experimental error or deviations when considered by one of ordinary skill in the art.

In some embodiments, Formula I, Form I has an XRPD pattern displaying at least two, at least three, at least four, at least five, or at least six of the degree 2θ-reflections with the greatest intensity as the XRPD pattern substantially as shown in FIG. 1.

In some embodiments, Formula I, Form I has an XRPD pattern comprising 2θ-reflections (+/−0.2 degrees 2θ) at 7.0°, 13.9°, and 27.9°. In some embodiments, Formula I, Form I has an XRPD pattern comprising 2θ-reflections (+/−0.2 degrees 2θ) at 7.0°, 13.9°, and 27.9°, and one, two or three of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 13.9°, and 27.9°, and one or two of the 2θ-reflections (+/−0.2 degrees 2θ) at 12.3°, 17.4°, and 24.6°. In some embodiments, Formula I, Form I has an XRPD pattern comprising 20-reflections (+/−0.2 degrees 2θ) at 7.0°, 13.9°, and 27.9°, and one of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 13.9°, and 27.9°, and two of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 12.3°, 13.9°, 17.4°, and 24.6°, and 27.9°. In some embodiments, Formula I, Form I has an XRPD pattern comprising any three 2θ-reflections (+/−0.2 degrees 2θ) selected from the group consisting of 7.0°, 12.3°, 13.9°, 17.4°, 24.6°, and 27.9°.

In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one, two, or three of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one or two of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and two of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising three of the 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least three of the 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least four of the 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least five of the 2θ-reflections (+/−0.2 degrees 2θ) at 7.0°, 12.3°, 13.9°, 17.4°, 21.4°, 23.2°, 24.6°, 27.9°, and 29.2°.

In some embodiments, Formula I, Form I has an XRPD pattern comprising at least three of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising at least four of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising at least five of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°.

In some embodiments, Formula I, Form I has an XRPD pattern comprising peaks at:

| Pos. | Rel. Int. |
|---|---|
| [°2Th.] | [%] |
| 7.0 | 100 |
| 12.3 | 44 |
| 13.9 | 57 |
| 15.9 | 9 |
| 16.7 | 10 |
| 17.1 | 34 |
| 17.4 | 40 |
| 20.1 | 8 |
| 21.4 | 12 |
| 22.6 | 4 |
| 23.2 | 18 |
| 24.6 | 41 |
| 25.6 | 7 |
| 26.3 | 5 |
| 26.7 | 7 |
| 27.8 | 42 |
| 27.9 | 76 |
| 29.2 | 20 |
| 31.2 | 4 |
| 35.0 | 12 |
| 36.6 | 2 |
| 37.4 | 2 |

In some embodiments, Formula I, Form I, is characterized by a DSC curve comprising an endothermic transition with an onset at about 192° C. In some embodiments, Formula I, Form I, is characterized by a DSC curve substantially as shown in FIG. 2.

In some embodiments, Formula I, Form I is unsolvated. In some embodiments, Formula I, Form I, is characterized by a TGA curve substantially as shown in FIG. 3.

In some embodiments, Formula I, Form I, is characterized by a DVS curve substantially as shown in FIG. 4. In some embodiments, Formula I, Form I, absorbs about 0.15% of water up to 95% RH at 25° C.

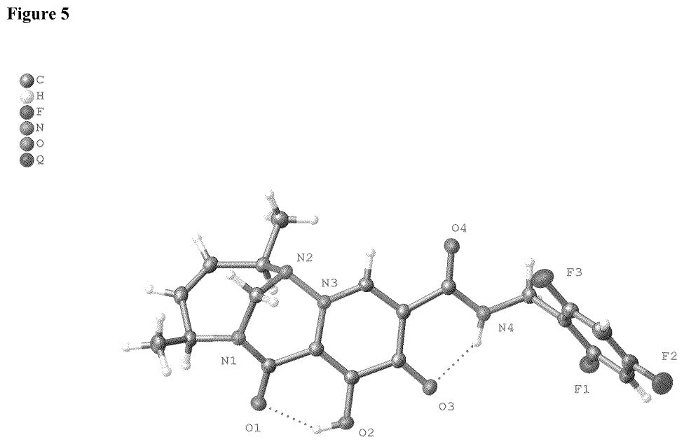

The single crystal data collected on Formula I, Form I are summarized in Table 1 below and also shown in FIG. 5. The crystal system of Formula I, Form I is monoclinic and the space group is P21. The cell parameters and calculated volume are: a=8.9146 (2) Å, b=8.6717 (2) Å, c=12.7101 (3) Å, α=90°, β=93.3170 (10)°, γ=90°, V=980.95 (4) Å³. The molecular weight is 448.40 g mol⁻¹ with Z=2, resulting in a calculated density of 1.518 g cm⁻³.

TABLE 1
Crystal Data and Data Collection Parameters for Formula I, Form I

| | |
|---|---|
| Empirical formula | C₂₁H₁₉F₃N₄O₄ |
| Formula weight (g mol⁻¹) | 448.40 |
| Temperature (K) | 100 |
| Wavelength (Å) | 1.54184 |
| Crystal system | monoclinic |
| Space group | P2₁ |
| Unit cell parameters | |
| a = 8.9146(2) Å | α = 90° |
| b = 8.6717(2) Å | β = 93.3170(10)° |
| c = 12.7107(3) Å | γ = 90° |
| Unit cell volume (Å³) | 980.95(4) |
| Cell formula units, Z | 2 |
| Calculated density (g cm⁻³) | 1.518 |
| Absorption coefficient (mm⁻¹) | 1.081 |
| F(000) | 464 |
| Crystal size (mm³) | 0.23 × 0.18 × 0.16 |
| Total reflections collected | 18571 |
| Index ranges | −10 ≤ h ≤ 10; −10 ≤ k ≤ 10; −15 ≤ l ≤ 15 |
| θ range for data collection | θₘᵢₙ = 3.483°, θₘₐₓ = 71.009° |
| Completeness to θ_{full} = 67.679° | 100.0% |
| Absorption correction | semi-empirical form equivalents |
| Max. and min. transmission | 0.7534 and 0.6029 |
| Refinement method | full matrix least-squares on F² |
| Independent reflections | 3768 [Rᵢₙₜ = 0.0262] |
| Reflections/restraints/parameters | 3768/2/297 |
| Goodness-of-fit on F² | S = 1.028 |
| Final residuals [I > 2σ(I)] | R = 0.0224, R_{w} = 0.0603 |
| Final residuals [all reflections] | R = 0.0225, R_{w} = 0.0605 |
| Absolute structure parameter | 0.05(3) |
| Extinction coefficient | 0.0044(5) |
| Largest diff. peak and hole (eÅ−3) | 0.155 and −0.122 |

### Formula I, Form II

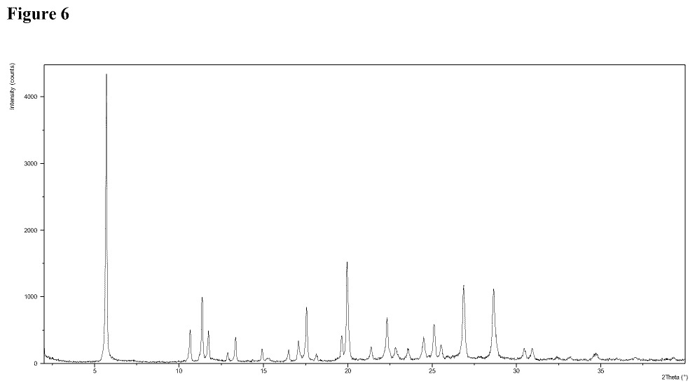

In some embodiments, the compound of Formula I is crystalline Form II (Formula I, Form II), wherein the crystal structure exhibits an X-ray powder diffraction (XRPD) pattern substantially as shown in FIG. 6.

In some embodiments, Formula I, Form II has an XRPD pattern displaying at least two, at least three, at least four, at least five, or at least six of the degree 2θ-reflections with the greatest intensity as the XRPD pattern substantially as shown in Figure. 6.

In some embodiments, Formula I, Form II has an XRPD pattern comprising 2θ-reflections (+/−0.2 degrees 2θ) at 5.7°, 19.9°, and 26.8°. In some embodiments, crystalline Formula I, Form II has an XRPD pattern comprising 2θ-reflections (+/−0.2 degrees 2θ) at 5.7°, 19.9°, and 26.8°, and one, two or three of the 0-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and one or two of the 2θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and one of the 2θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and two of the 2θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°. In some embodiments, crystalline Formula I, Form II has an XRPD pattern comprising any three 2θ-reflections (+/−0.2 degrees 2θ) selected from the group consisting of 5.7°, 11.4°, 17.6°, 19.9°, 26.8°, and 28.6°.

In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one, two or three of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one or two of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and two of the 2θ-reflections (+/−0.2 degrees 2θ) at 10.7°, 22.3°, and 25.1°. In some embodiments, crystalline Formula I, Form II has an XRPD pattern comprising any three 2θ-reflections (+/−0.2 degrees 2θ) selected from the group consisting of 5.7°, 10.7°, 11.4°, 17.6°, 19.9°, 22.3°, 25.1°, 26.8°, and 28.6°. In some embodiments, crystalline Formula I, Form II has an XRPD pattern comprising 2θ-reflections (+/−0.2 degrees 2θ) at 5.7°, 10.7°, 11.4°, 17.6°, 19.9°, 22.3°, 25.1°, 26.8°, and 28.6°.

In some embodiments, crystalline Formula I, Form II has an XRPD pattern comprising at least three of 2θ-reflections (+/−0.2 degrees 2θ) selected from the group consisting of 5.7°, 10.7°, 11.4°, 17.6°, 19.9°, 22.3°, 25.1°, 26.8°, and 28.6°. In some embodiments, crystalline Formula I, Form II has an XRPD pattern comprising at least four of 2θ-reflections (+/−0.2 degrees 2θ) selected from the group consisting of 5.7°, 10.7°, 11.4°, 17.6°, 19.9°, 22.3°, 25.1°, 26.8°, and 28.6°. In some embodiments, crystalline Formula I, Form II has an XRPD pattern comprising at least five of 2θ-reflections (+/−0.2 degrees 2θ) selected from the group consisting of 5.7°, 10.7°, 11.4°, 17.6°, 19.9°, 22.3°, 25.1°, 26.8°, and 28.6°.

In some embodiments, crystalline Formula I, Form II has an XRPD pattern comprising at least three of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°. In some embodiments, crystalline Formula I, Form II has an XRPD pattern comprising at least four of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°. In some embodiments, crystalline Formula I, Form II has an XRPD pattern comprising at least five of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°.

In some embodiments, Formula I, Form II has an XRPD pattern comprising peaks at:

| Pos. | Rel. Int. |
|---|---|
| [°2Th.] | [%] |
| 5.7 | 100 |
| 10.7 | 11 |
| 11.4 | 23 |
| 11.7 | 11 |
| 13.3 | 8 |
| 14.9 | 4 |
| 16.5 | 3 |
| 17.1 | 7 |
| 17.6 | 19 |
| 19.6 | 9 |
| 19.9 | 35 |
| 21.4 | 4 |
| 22.3 | 14 |
| 22.9 | 3 |
| 23.6 | 3 |
| 24.5 | 7 |
| 25.1 | 12 |
| 25.5 | 5 |
| 26.8 | 25 |
| 28.6 | 25 |
| 30.5 | 4 |
| 30.9 | 4 |
| 34.7 | 2 |

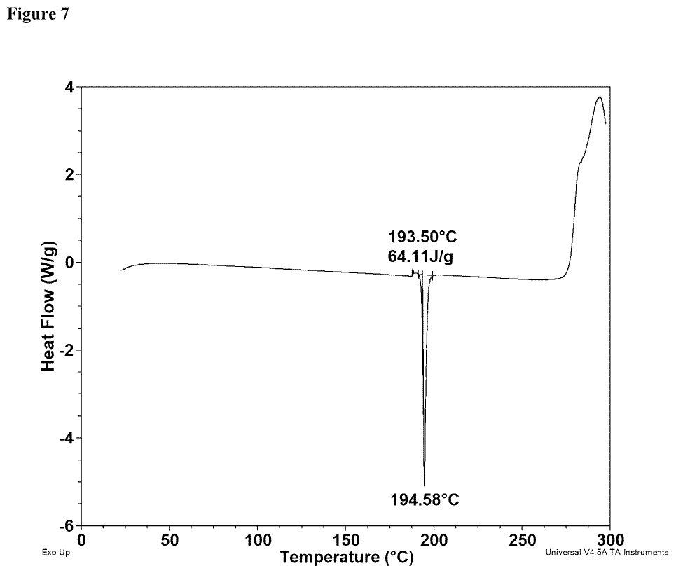

In some embodiments, Formula I, Form II, is characterized by a DSC curve comprising an endothermic transition with an onset at about 194° C. In some embodiments, Formula I, Form II, is characterized by a DSC curve substantially as shown in FIG. 7.

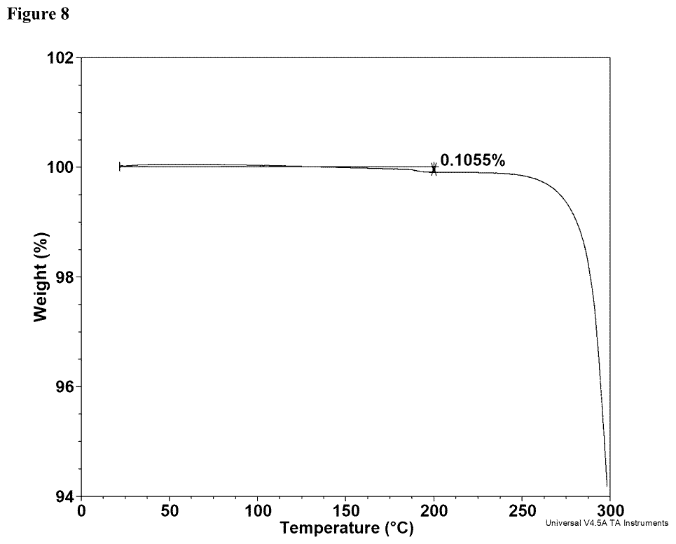

In some embodiments, Formula I, Form II is unsolvated. In some embodiments, Formula I, Form II, is characterized by a TGA curve substantially as shown in FIG. 8.

### Sodium Salt of the Compound of Formula I

In some embodiments, the pharmaceutical composition comprises a sodium salt of the compound of Formula I which can be amorphous or crystalline. In some embodiments, the sodium salt of the compound of Formula I is crystalline.

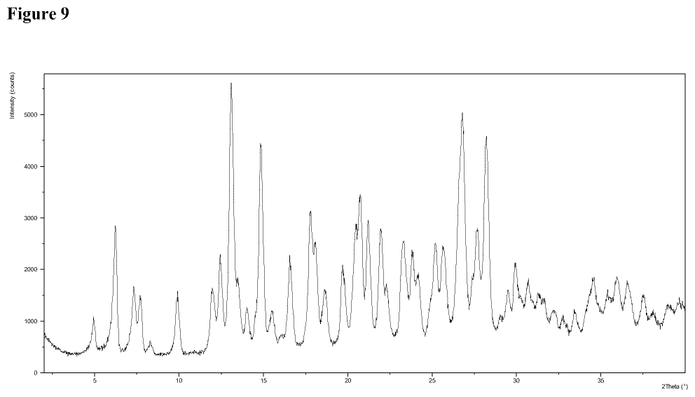

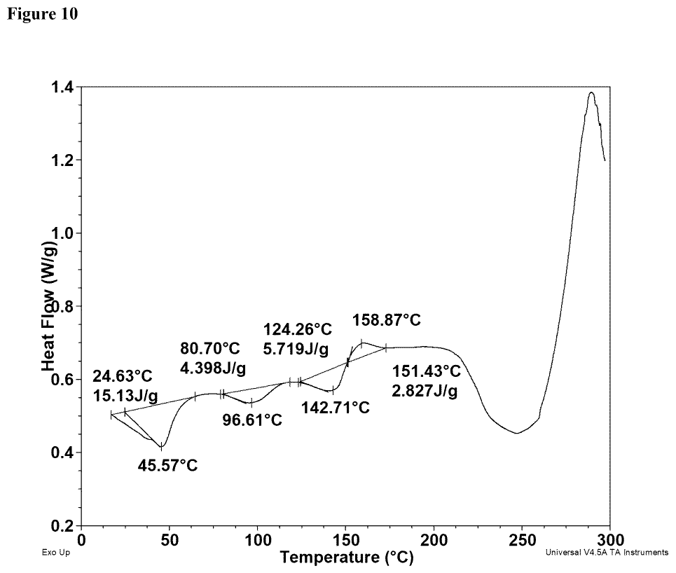

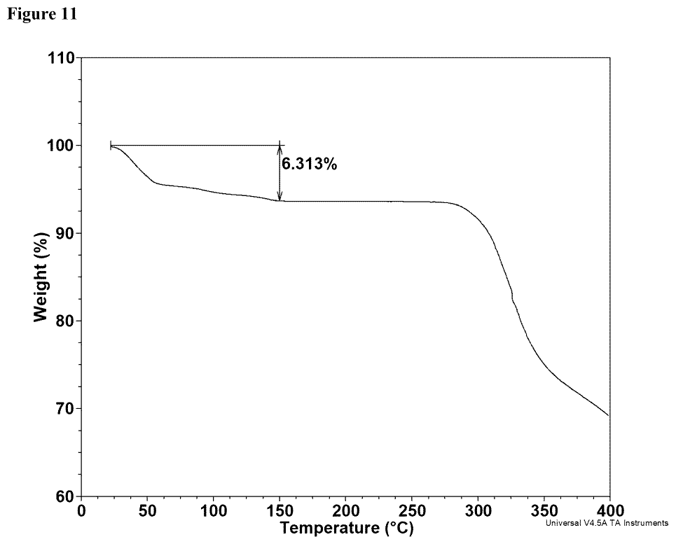

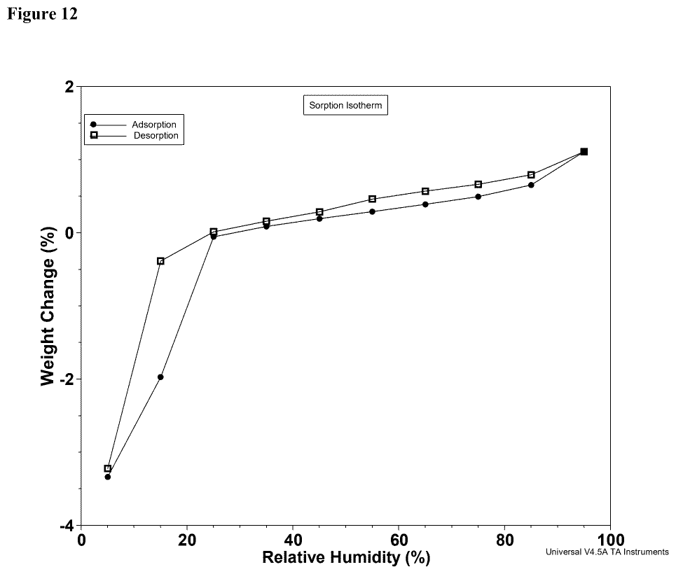

In some embodiments, the crystalline sodium salt of the compound of Formula I is a Form I (“compound of Formula I, sodium salt, Form I” or “Formula I, sodium salt, Form I”) having an XRPD profile substantially as shown in FIG. 9. Compound of Formula I, sodium salt, Form I may exhibit a DSC thermogram substantially as shown in FIG. 10. Compound of Formula I, sodium salt, Form I may exhibit a TGA graph substantially as shown in FIG. 11. Compound of Formula I, sodium salt, Form I may exhibit a DVS curve substantially as shown in FIG. 12.

In some embodiments, Formula I, sodium salt, Form I has an XRPD pattern displaying at least two, at least three, at least four, at least five, or at least six of the degree 2θ-reflections with the greatest intensity as the XRPD pattern substantially as shown in FIG. 9.

In some embodiments, the Formula I, sodium salt, Form I has an XRPD pattern comprising 2θ-reflections (+/−0.2 degrees 2θ) at 13.1°, 14.8°, and 26.8°. In some embodiments, the Formula I, sodium salt, Form I has an XRPD pattern comprising 2θ-reflections (+/−0.2 degrees 2θ) at 13.1°, 14.8°, and 26.8°, and one, two or three of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.1°, 14.8°, and 26.8°, and one or two of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.1°, 14.8°, and 26.8°, and one of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.1°, 14.8°, and 26.8°, and two of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.1°, 14.8°, 17.8°, 20.7°, 26.8°, and 28.2°. In some embodiments, the Formula I, sodium salt, Form I has an XRPD pattern comprising any three of the 2θ-reflections (+/−0.2 degrees 2θ) at 13.1°, 14.8°, 17.8°, 20.7°, 26.8°, and 28.2°.

In some embodiments, the Formula I, sodium salt, Form I has an XRPD pattern comprising 2θ-reflections (+/−0.2 degrees 2θ) at 13.1°, 14.8°, 17.8°, 20.7°, 26.8°, and 28.2°, and one, two or three of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.1°, 14.8°, 17.8°, 20.7°, 26.8°, and 28.2°, and one or two of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.1°, 14.8°, 17.8°, 20.7°, 26.8°, and 28.2°, and one of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.1°, 14.8°, 17.8°, 20.7°, 26.8°, and 28.2°, and two of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 6.2°, 13.1°, 14.8°, 17.8°, 20.7°, 21.2°, 23.3°, 26.8°, and 28.2°. In some embodiments, the Formula I, sodium salt, Form I has an XRPD pattern comprising any three of the 2θ-reflections (+/−0.2 degrees 2θ) at 6.2°, 13.1°, 14.8°, 17.8°, 20.7°, 21.2°, 23.3°, 26.8°, and 28.2°.

In some embodiments, the Formula I, sodium salt, Form I has an XRPD pattern comprising at least three of the 2θ-reflections (+/−0.2 degrees 2θ) at 6.2°, 13.1°, 14.8°, 17.8°, 20.7°, 21.2°, 23.3°, 26.8°, and 28.2°. In some embodiments, the Formula I, sodium salt, Form I has an XRPD pattern comprising at least four of the 2θ-reflections (+/−0.2 degrees 2θ) at 6.2°, 13.1°, 14.8°, 17.8°, 20.7°, 21.2°, 23.3°, 26.8°, and 28.2°. In some embodiments, the Formula I, sodium salt, Form I has an XRPD pattern comprising at least five of the 2θ-reflections (+/−0.2 degrees 2θ) at 6.2°, 13.1°, 14.8°, 17.8°, 20.7°, 21.2°, 23.3°, 26.8°, and 28.2°.

In some embodiments, the Formula I, sodium salt, Form I has an XRPD pattern comprising at least three of the 2θ-reflections (+/−0.2 degrees 2θ) at 6.2°, 12.4°, 13.1°, 14.8°, 16.6°, 17.8°, 20.7°, 21.2°, 21.9°, 23.3°, 25.2°, 26.8°, and 28.2°. In some embodiments, the Formula I, sodium salt, Form I has an XRPD pattern comprising at least four of the 2θ-reflections (+/−0.2 degrees 2θ) at 6.2°, 12.4°, 13.1°, 14.8°, 16.6°, 17.8°, 20.7°, 21.2°, 21.9°, 23.3°, 25.2°, 26.8°, and 28.2°. In some embodiments, the Formula I, sodium salt, Form I has an XRPD pattern comprising at least five of the 2θ-reflections (+/−0.2 degrees 2θ) at 6.2°, 12.4°, 13.1°, 14.8°, 16.6°, 17.8°, 20.7°, 21.2°, 21.9°, 23.3°, 25.2°, 26.8°, and 28.2°.

In some embodiments, the Formula I, sodium salt, Form I has an XRPD pattern

| Pos. | Rel. Int. |
|---|---|
| [°2Th.] | [%] |
| 4.9 | 11 |
| 6.2 | 47 |
| 7.7 | 21 |
| 9.9 | 22 |
| 11.9 | 22 |
| 12.4 | 34 |
| 13.1 | 100 |
| 14.8 | 76 |
| 16.6 | 32 |
| 17.8 | 50 |
| 18.7 | 20 |
| 19.7 | 27 |
| 20.7 | 54 |
| 21.2 | 43 |
| 21.9 | 40 |
| 23.3 | 35 |
| 25.2 | 32 |
| 25.6 | 28 |
| 26.8 | 79 |
| 28.2 | 73 |
| 29.9 | 23 |
| 36.6 | 15 |

In some embodiments, the Formula I, sodium salt, Form I is characterized by a DSC thermogram substantially as shown in FIG. 10.

In some embodiments, the Formula I, sodium salt, Form I is characterized by a DSC thermogram having one or more of (i) an endothermic transition at 25° C., (ii) an endothermic transition at 81° C., (iii) an endothermic transition at 124° C., and (iv) an exothermic transition at 151° C. In some embodiments, the Formula I, sodium salt, Form I is characterized by a DSC thermogram having (i) an endothermic transition at 25° C., (ii) an endothermic transition at 81° C., (iii) an endothermic transition at 124° C., and (iv) an exothermic transition at 151° C.

In some embodiments, the Formula I, sodium salt, Form I is characterized by a TGA curve substantially as shown in FIG. 11.

In some embodiments, the Formula I, sodium salt, Form I is characterized by a DVS curve substantially as shown in shown in FIG. 12.

In some embodiments, the Formula I, sodium salt, Form I absorbs about 4% of water up to 95% RH at 25° C.

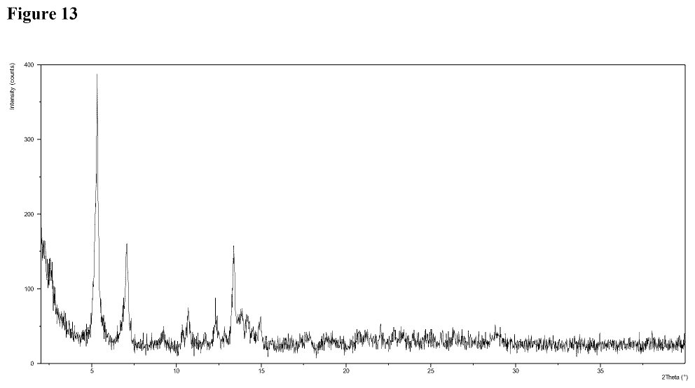

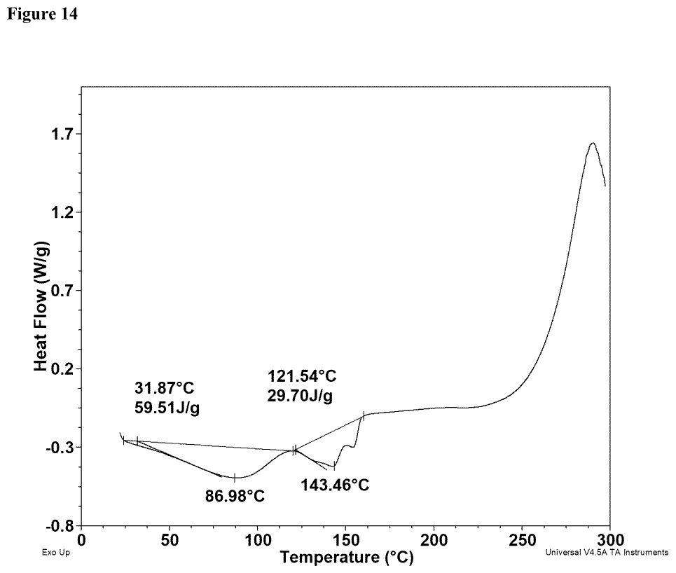

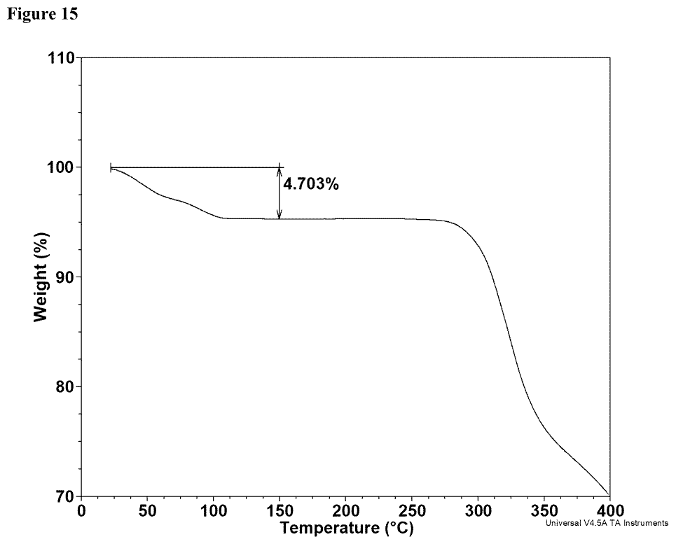

In some embodiments, the crystalline sodium salt of the compound of Formula I is a Form II (“compound of Formula I, sodium salt, Form II” or “Formula I, sodium salt, Form II”) having an XRPD profile substantially as shown in FIG. 13. Compound of Formula I, sodium salt, Form II may exhibit a DSC thermogram substantially as shown in FIG. 14. Compound of Formula I, sodium salt, Form II may exhibit a TGA graph substantially as shown in FIG. 15.

In some embodiments, the Formula I, sodium salt, Form II has an XRPD pattern comprising 2θ-reflections (+/−0.2 degrees 2θ) at 5.3°, 7.1°, and 13.4°. In some embodiments, the Formula I, sodium salt, Form II has an XRPD pattern comprising 2θ-reflections (+/−0.2 degrees 2θ) at 5.3°, 7.1°, 10.7°, and 13.4°.

In some embodiments, the Formula I, sodium salt, Form II has an XRPD pattern comprising peaks at:

| Pos. | Rel. Int. |
|---|---|
| [°2Th.] | [%] |
| 5.3 | 100 |
| 7.1 | 44 |
| 10.7 | 15 |
| 13.4 | 41 |

In some embodiments, the Formula I, sodium salt, Form II is characterized by a DSC thermogram substantially as shown in FIG. 14.

In some embodiments, the Formula I, sodium salt, Form II is characterized by a DSC thermogram having one or both of (i) an endothermic transition at about 32° C. and (ii) an endothermic transition at about 122° C. In some embodiments, the Formula I, sodium salt, Form I is characterized by a DSC thermogram having (i) an endothermic transition at about 32° C. and (ii) an endothermic transition at about 122° C.

In some embodiments, the Formula I, sodium salt, Form II is characterized by a TGA curve substantially as shown in FIG. 15.

In some embodiments, the Formula I, sodium salt, Form II loses mass starting at about ambient temperature.

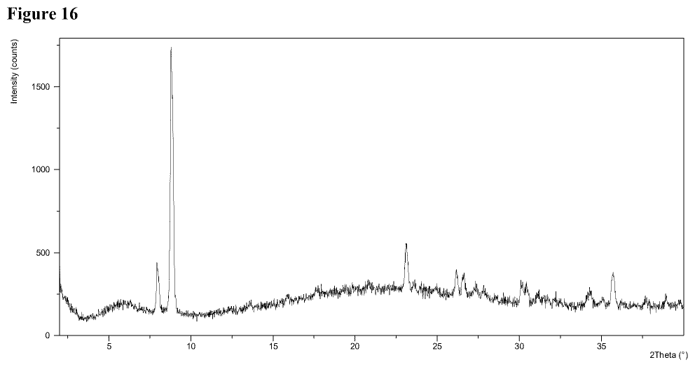

In some embodiments, the crystalline sodium salt of the compound of Formula I is a Form III (“compound of Formula I, sodium salt, Form III” or “Formula I, sodium salt, Form III”). In some embodiments, Compound of Formula I, sodium salt, Form III has an XRPD profile substantially as shown in FIG. 16.

In some embodiments, Formula I, sodium salt, Form III has an XRPD pattern displaying at least two, at least three, at least four, at least five, or at least six of the degree 2θ-reflections with the greatest intensity as the XRPD pattern substantially as shown in FIG. 16.

In some embodiments, the Formula I, sodium salt, Form III has an XRPD pattern comprising 2θ-reflections (+/−0.2 degrees 2θ) at 7.9°, 8.8°, and 23.1°. In some embodiments, the Formula I, sodium salt, Form III has an XRPD pattern comprising 2θ-reflections (+/−0.2 degrees 2θ) at 7.9°, 8.8°, and 23.1, and one, two or three of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and one or two of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and one of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and two of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, 23.1°, 26.2°, 26.6°, and 30.3°. In some embodiments, the Formula I, sodium salt, Form I has an XRPD pattern comprising any three of the 2θ-reflections (+/−0.2 degrees 2θ) at 7.9°, 8.8°, 23.1°, 26.2°, 26.6°, and 30.3°.

In some embodiments, the Formula I, sodium salt, Form III has an XRPD pattern comprising at least three of the 2θ-reflections (+/−0.2 degrees 2θ) at 7.9°, 8.8°, 23.1°, 26.2°, 26.6°, and 30.3°. In some embodiments, the Formula I, sodium salt, Form III has an XRPD pattern comprising at least four of the 2θ-reflections (+/−0.2 degrees 2θ) at 7.9°, 8.8°, 23.1°, 26.2°, 26.6°, and 30.3°. In some embodiments, the Formula I, sodium salt, Form III has an XRPD pattern comprising at least five of the 2θ-reflections (+/−0.2 degrees 2θ) at 7.9°, 8.8°, 23.1°, 26.2°, 26.6°, and 30.3°.

In some embodiments, the Formula I, sodium salt, Form III has an XRPD pattern comprising peaks at:

| Pos. | Rel. Int. |
|---|---|
| [°2Th.] | [%] |
| 7.9 | 18 |
| 8.8 | 100 |
| 23.1 | 17 |
| 26.2 | 9 |
| 26.6 | 7 |
| 30.3 | 5 |

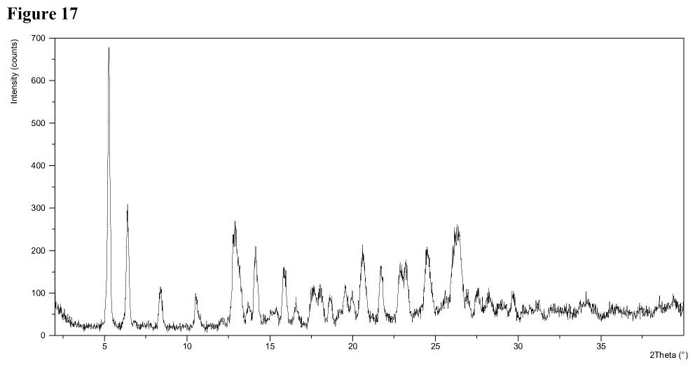

In some embodiments, the crystalline sodium salt of the compound of Formula I is a Form IV (“compound of Formula I, sodium salt, Form IV” or “Formula I, sodium salt, Form IV”). In some embodiments, the compound of Formula I, sodium salt, Form IV has an XRPD profile substantially as shown in FIG. 17.

In some embodiments, Formula I, sodium salt, Form IV has an XRPD pattern displaying at least two, at least three, at least four, at least five, or at least six of the degree 2θ-reflections with the greatest intensity as the XRPD pattern substantially as shown in FIG. 17.

In some embodiments, the Formula I, sodium salt, Form IV has an XRPD pattern comprising 2θ-reflections (+/−0.2 degrees 2θ) at 5.3°, 6.4°, and 12.8°. In some embodiments, the Formula I, sodium salt, Form IV has an XRPD pattern comprising 2θ-reflections (+/−0.2 degrees 2θ) at 5.3°, 6.4°, and 12.8°, and one, two or three of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and one or two of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and one of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and two of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°. In some embodiments, the Formula I, sodium salt, Form IV has an XRPD pattern comprising any three of the 2θ-reflections (+/−0.2 degrees 2θ) at 5.3°, 6.4°, 12.8°, 14.1°, 24.5°, and 26.4°.

In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one, two, or three of the 2θ-reflections (+/−0.2 degrees 2θ) at 8.4°, 15.8°, and 20.7°. In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one or two of the 2θ-reflections (+/−0.2 degrees 2θ) at 8.4°, 15.8°, and 20.7°. In some embodiments, Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one of the 2θ-reflections (+/−0.2 degrees 2θ) at 8.4°, 15.8°, and 20.7°. In some embodiments, Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and two of the 2θ-reflections (+/−0.2 degrees 2θ) at 8.4°, 15.8°, and 20.7°. In some embodiments, the Formula I, sodium salt, Form IV has an XRPD pattern comprising 2θ-reflections (+/−0.2 degrees 2θ) at 5.3°, 6.4°, 8.4°, 12.8°, 14.1°, 15.8°, 20.7°, 24.5°, and 26.4°. In some embodiments, the Formula I, sodium salt, Form IV has an XRPD pattern comprising any three of the 2θ-reflections (+/−0.2 degrees 2θ) at 5.3°, 6.4°, 8.4°, 12.8°, 14.1°, 15.8°, 20.7°, 24.5°, and 26.4°.

In some embodiments, the Formula I, sodium salt, Form IV has an XRPD pattern comprising at least three of the 2θ-reflections (+/−0.2 degrees 2θ) at 5.3°, 6.4°, 8.4°, 12.8°, 14.1°, 15.8°, 20.7°, 24.5°, and 26.4°. In some embodiments, the Formula I, sodium salt, Form IV has an XRPD pattern comprising at least four of the 2θ-reflections (+/−0.2 degrees 2θ) at 5.3°, 6.4°, 8.4°, 12.8°, 14.1°, 15.8°, 20.7°, 24.5°, and 26.4°. In some embodiments, the Formula I, sodium salt, Form IV has an XRPD pattern comprising at least five of the 2θ-reflections (+/−0.2 degrees 2θ) at 5.3°, 6.4°, 8.4°, 12.8°, 14.1°, 15.8°, 20.7°, 24.5°, and 26.4°.

In some embodiments, the Formula I, sodium salt, Form IV has an XRPD pattern comprising at least three of the 2θ-reflections (+/−0.2 degrees 2θ) 5.3°, 6.4°, 8.4°, 12.8°, 14.1°, 15.8°, 20.7°, 21.7°, 22.8°, 23.2°, 24.5°, and 26.4°. In some embodiments, the Formula I, sodium salt, Form IV has an XRPD pattern comprising at least four of the 2θ-reflections (+/−0.2 degrees 2θ) at 5.3°, 6.4°, 8.4°, 12.8°, 14.1°, 15.8°, 20.7°, 21.7°, 22.8°, 23.2°, 24.5°, and 26.4°. In some embodiments, the Formula I, sodium salt, Form IV has an XRPD pattern comprising at least five of the 2θ-reflections (+/−0.2 degrees 2θ) at 5.3°, 6.4°, 8.4°, 12.8°, 14.1°, 15.8°, 20.7°, 21.7°, 22.8°, 23.2°, 24.5°, and 26.4°.

In some embodiments, the Formula I, sodium salt, Form IV has an XRPD pattern comprising peaks at:

| Pos. | Rel. Int. |
|---|---|
| [°2Th.] | [%] |
| 5.3 | 100 |
| 6.4 | 41 |
| 8.4 | 13 |
| 10.5 | 11 |
| 12.8 | 30 |
| 14.1 | 24 |
| 15.8 | 18 |
| 17.5 | 11 |
| 18.6 | 8 |
| 19.4 | 6 |
| 20.7 | 21 |
| 21.7 | 17 |
| 22.8 | 17 |
| 23.2 | 17 |
| 24.5 | 23 |
| 26.4 | 27 |
| 27.5 | 8 |
| 29.7 | 7 |

### Potassium Salt of the Compound of Formula I

In some embodiments, the composition comprises a potassium salt of the compound of Formula I which can be amorphous or crystalline. In some embodiments, the potassium salt of the compound of Formula I is crystalline.

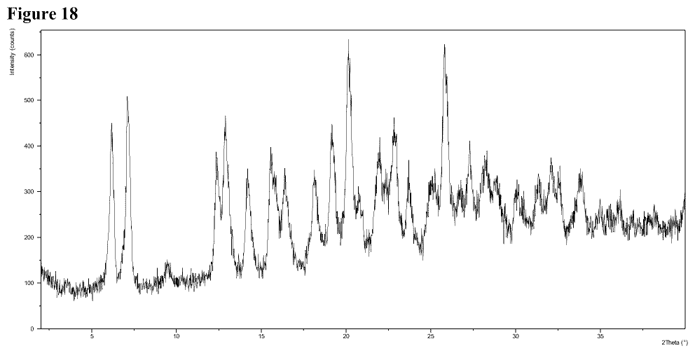

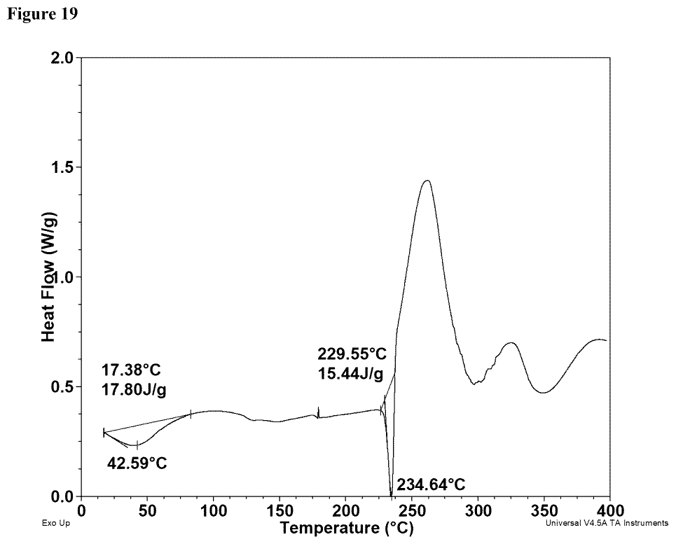

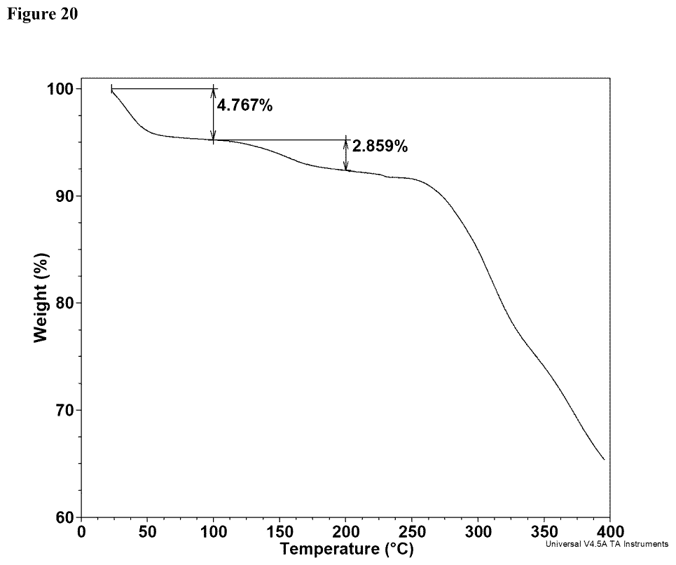

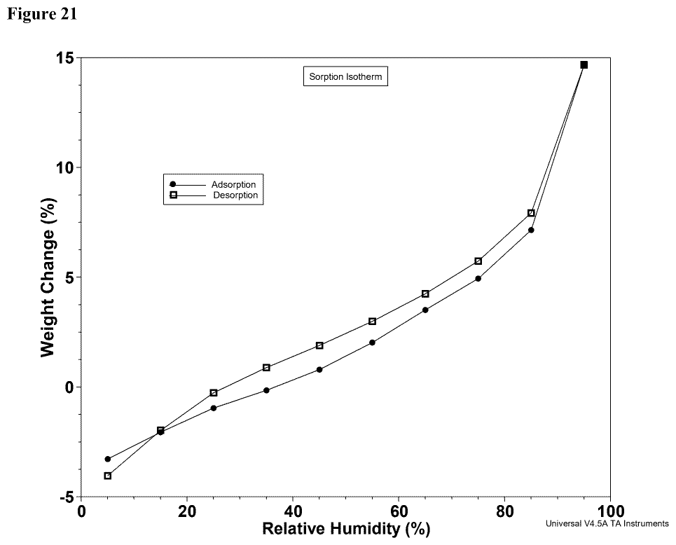

In some embodiments, the crystalline potassium salt of the compound of Formula I is the Form I (“compound of Formula I, potassium salt, Form I” or “Formula I, potassium salt, Form I”). In some embodiments, Formula I, potassium salt, Form I has an XRPD profile substantially as shown in FIG. 18. Formula I, potassium salt, Form I may exhibit a DSC thermogram substantially as shown in FIG. 19. Formula I, potassium salt, Form I may exhibit a TGA graph substantially as shown in FIG. 20. Formula I, potassium salt, Form I may exhibit a DVS curve substantially as shown in FIG. 21.

In some embodiments, Formula I, potassium salt, Form I has an XRPD pattern displaying at least two, at least three, at least four, at least five, or at least six of the degree 2θ-reflections with the greatest intensity as the XRPD pattern substantially as shown in FIG. 18.

In some embodiments, Formula I, potassium salt, Form I has an XRPD pattern comprising 2θ-reflections (+/−0.2 degrees 2θ) at 7.1°, 20.1°, and 25.8°. In some embodiments, Formula I, potassium salt, Form I has an XRPD pattern comprising 2θ-reflections (+/−0.2 degrees 2θ) at 7.1°, 20.1°, and 25.8°, and one, two or three of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and one or two of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and one of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and two of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°. In some embodiments, Formula I, potassium salt, Form I has an XRPD pattern comprising any three of the 2θ-reflections (+/−0.2 degrees 2θ) at 6.2°, 7.1°, 13.0°, 19.2°, 20.1°, and 25.8°.

In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one, two or three of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one or two of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and two of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 14.2°, 15.5°, 19.2°, 20.1°, 22.9°, and 25.8°. In some embodiments, Formula I, potassium salt, Form I has an XRPD pattern comprising any three of the 2θ-reflections (+/−0.2 degrees 2θ) at 6.2°, 7.1°, 13.0°, 14.2°, 15.5°, 19.2°, 20.1°, 22.9°, and 25.8°.

In some embodiments, Formula I, potassium salt, Form I has an XRPD pattern comprising at least three of the 2θ-reflections (+/−0.2 degrees 2θ) at 6.2°, 7.1°, 13.0°, 14.2°, 15.5°, 16.4°, 18.1°, 19.2°, 20.1°, 21.9°, 22.9°, and 25.8°. In some embodiments, Formula I, potassium salt, Form I has an XRPD pattern comprising at least four of the 2θ-reflections (+/−0.2 degrees 2θ) at 6.2°, 7.1°, 13.0°, 14.2°, 15.5°, 16.4°, 18.1°, 19.2°, 20.1°, 21.9°, 22.9°, and 25.8°. In some embodiments, Formula I, potassium salt, Form I has an XRPD pattern comprising at least five of the 2θ-reflections (+/−0.2 degrees 2θ) at 6.2°, 7.1°, 13.0°, 14.2°, 15.5°, 16.4°, 18.1°, 19.2°, 20.1°, 21.9°, 22.9°, and 25.8°.

In some embodiments, Formula I, potassium salt, Form I has an XRPD pattern

| Pos. | Rel. Int. |
|---|---|
| [°2Th.] | [%] |
| 6.2 | 85 |
| 7.1 | 100 |
| 13.0 | 60 |
| 14.2 | 47 |
| 15.5 | 57 |
| 16.4 | 45 |
| 18.1 | 45 |
| 19.2 | 63 |
| 20.1 | 98 |
| 21.9 | 45 |
| 22.9 | 55 |
| 23.7 | 28 |
| 25.8 | 90 |
| 28.6 | 21 |
| 32.1 | 29 |
| 33.8 | 25 |

In some embodiments, Formula I, potassium salt, Form I is characterized by a DSC thermogram substantially as shown in FIG. 19.

In some embodiments, Formula I, potassium salt, Form I is characterized by a DSC thermogram having one or both of (i) an endothermic transition at about 17° C. and (ii) comprises an endothermic transition at about 230° C. In some embodiments, Formula I, potassium salt, Form I is characterized by a DSC thermogram having (i) an endothermic transition at about 17° C. and (ii) comprises an endothermic transition at about 230° C.

In some embodiments, Formula I, potassium salt, Form I is characterized by a TGA curve substantially as shown in FIG. 20. In some embodiments, Formula I, potassium salt, Form I loses mass in multiple stages with one stage starting at about ambient temperature and the next stage starting at about 100° C.

In some embodiments, Formula I, potassium salt, Form I is characterized by a DVS curve substantially as shown in shown in FIG. 21. In some embodiments, Formula I, potassium salt, Form I absorbs more than about 18% of water up to 95% RH at 25° C.

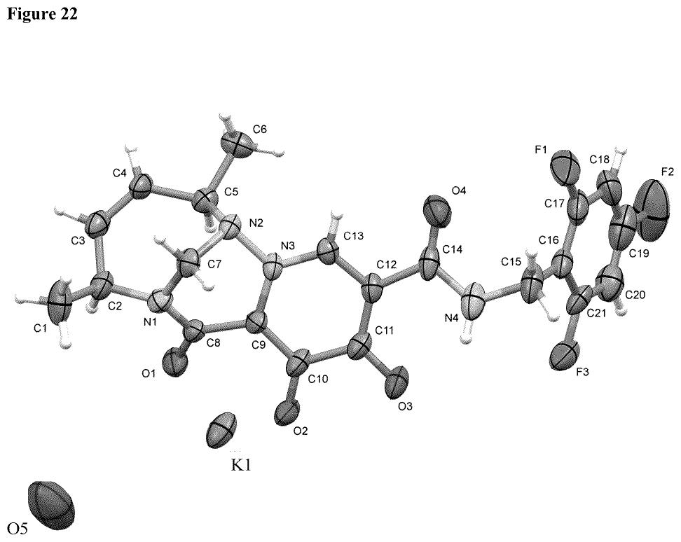

The single crystal data collected on Formula I, potassium salt, Form I are summarized in Table 2 below and also shown in FIG. 22. The crystal system is trigonal and the space group is P3221. The cell parameters and calculated volume are: a=28.6496 (4) Å, b=28.6496 (4) Å, c=6.89340 (10) Å, α=90°, β=90°, γ=120°, V=4900.06 (15) Å³. The molecular weight is 494.49 g mol⁻¹ with Z=6, resulting in a calculated density of 1.005 g cm⁻³.

TABLE 2
Crystal Data and Data Collection Parameters
for Formula I, Potassium Salt, Form I

| Empirical formula | C21H18F3KN4O4.50 | |
|---|---|---|
| Formula weight (g mol − 1) | 494.49 | |
| Temperature (K) | 150 | (2) |
| Wavelength (Å) | 1.54184 | |
| Crystal system | trigonal | |
| Space group | P3₂21 | |
| Unit cell parameters | a = 28.6496(4) Å α = 90° | |
| | b = 28.6496(4) Å β = 90° | |
| | c = 6.89340(10) Å γ = 120° | |
| Unit cell volume (Å3) | 4900.06 | (15) |

| | |
|---|---|
| Cell formula units, Z | 6 |
| Calculated density (g cm − 3) | 1.005 |
| Absorption coefficient (mm − 1) | 1.819 |
| F(000) | 1524 |
| Crystal size (mm3) | 0.37 × 0.1 × 0.02 |
| Reflections used for cell measurement | 16195 |
| θ range for cell measurement | 3.5560°-75.3450° |
| Total reflections collected | 33594 |
| Index ranges | −35 ≤ h ≤ 33; −34 ≤ k ≤ 24; −8 ≤ l ≤ 8 |
| θ range for data collection | θmin = 3.563°, θmax = 76.309° |
| Completeness to θmax | 98.7% |
| Completeness to θfull = 67.684° | 99.9% |
| Absorption correction | multi-scan |
| Transmission coefficient range | 0.692-1.000 |
| Refinement method | full matrix least-squares on F2 |
| Independent reflections | 6746 [Rint = 0.0379, Rσ = 0.0262] |
| Reflections [I > 2σ(I)] | 6485 |
| Reflections/restraints/parameters | 6746/0/305 |
| Goodness-of-fit on F2 | S = 1.06 |
| Final residuals [I > 2σ(I)] | R = 0.0672, Rw = 0.1972 |
| Final residuals [all reflections] | R = 0.0691, Rw = 0.1998 |
| Largest diff. peak and hole (e Å − 3) | 0.359, −0.324 |
| Max/mean shift/standard uncertainty | 0.000/0.000 |
| Absolute structure determination | Flack parameter: 0.0167(7) |
| | Hooft parameter: 0.103(5) |
| | Friedel coverage: 97.2% |

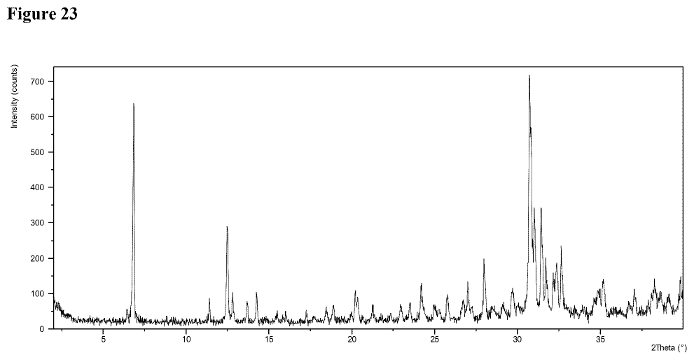

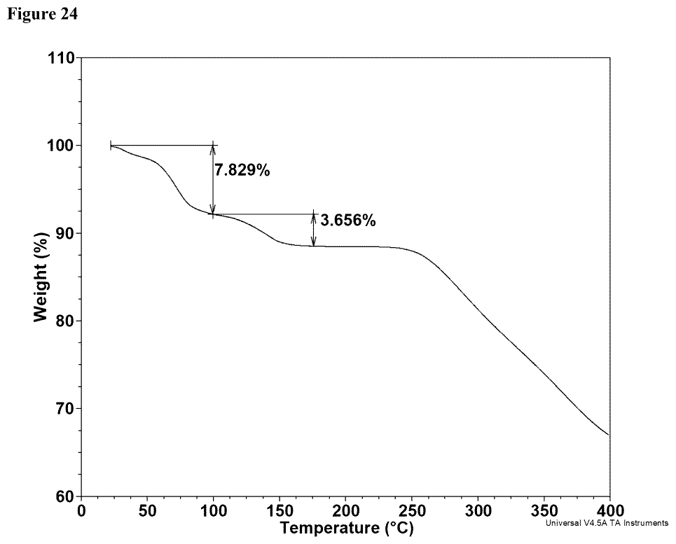

In some embodiments, the crystalline potassium salt of the compound of Formula I is the Form II (“compound of Formula I, potassium salt, Form II” or “Formula I, potassium salt, Form II”). In some embodiments, Formula I, potassium salt, Form II has an XRPD profile substantially as shown in FIG. 23. Compound of Formula I, potassium salt, Form II may exhibit a TGA curve substantially as shown in FIG. 24.

In some embodiments, Formula I, potassium salt, Form II has an XRPD pattern displaying at least two, at least three, at least four, at least five, or at least six of the degree 2θ-reflections with the greatest intensity as the XRPD pattern substantially as shown in FIG. 23.

In some embodiments, Formula I, potassium salt, Form II has an XRPD pattern comprising 2θ-reflections (+/−0.2 degrees 2θ) at 6.8°, 30.7°, and 31.4°. In some embodiments, Formula I, potassium salt, Form II has an XRPD pattern comprising 2θ-reflections (+/−0.2 degrees 2θ) at 6.8°, 30.7°, and 31.4°, and one, two or three of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 30.7°, and 31.4°, and one or two of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 30.7°, and 31.4°, and one of the degree 2θ-reflections (+/−0.2 degrees 2θ) at 12.5°, 28.0°, and 32.6°. In some embodiments, Formula I, potassium salt, Form II has an XRPD pattern comprising degree 2θ-reflections (+/−0.2 degrees 2θ) at 6.8°, 30.7°, and 31.4°, and two of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°. In some embodiments, Formula I, potassium salt, Form II has an XRPD pattern comprising any three of the 2θ-reflections (+/−0.2 degrees 2θ) at 6.8°, 12.5°, 28.0°, 30.7°, 31.4°, and 32.6°.

In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one, two or three of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one or two of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and two of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 14.3°, 24.2°, 25.7°, 28.0°, 30.7°, 31.4°, and 32.6°. In some embodiments, Formula I, potassium salt, Form II has an XRPD pattern comprising any three of the 2θ-reflections (+/−0.2 degrees 2θ) at 6.8°, 12.5°, 14.3°, 24.2°, 25.7°, 28.0°, 30.7°, 31.4°, and 32.6°.

In some embodiments, Formula I, potassium salt, Form II has an XRPD pattern comprising at least three of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°. In some embodiments, Formula I, potassium salt, Form II has an XRPD pattern comprising at least four of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°. In some embodiments, Formula I, potassium salt, Form II has an XRPD pattern comprising at least five of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°.

In some embodiments, Formula I, potassium salt, Form II has an XRPD pattern

| Pos. | Rel. Int. |
|---|---|
| [°2Th.] | [%] |
| 6.8 | 94 |
| 12.5 | 41 |
| 14.3 | 11 |
| 20.3 | 8 |
| 24.2 | 14 |
| 25.7 | 10 |
| 26.9 | 6 |
| 28.0 | 21 |
| 29.7 | 10 |
| 30.7 | 100 |
| 31.4 | 43 |
| 32.3 | 12 |
| 32.6 | 23 |
| 35.0 | 9 |

In some embodiments, Formula I, potassium salt, Form II is characterized by a TGA curve substantially as shown in FIG. 24. In some embodiments, Formula I, potassium salt, Form II loses mass in multiple stages with one stage starting at about ambient temperature and the next stage starting at about 100° C.

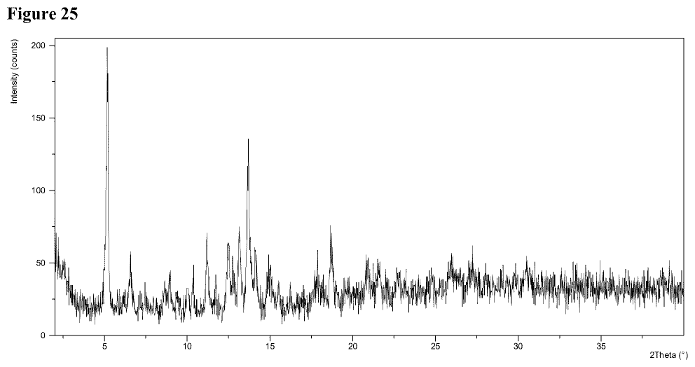

In some embodiments, the crystalline potassium salt of the compound of Formula I is the Form III (“compound of Formula I, potassium salt, Form III” or “Formula I, potassium salt, Form III”). In some embodiments, Formula I, potassium salt, Form III has an XRPD profile substantially as shown in FIG. 25.

In some embodiments, Formula I, potassium salt, Form III has an XRPD pattern displaying at least two, at least three, at least four, at least five, or at least six of the degree 2θ-reflections with the greatest intensity as the XRPD pattern substantially as shown in FIG. 25.

In some embodiments, Formula I, potassium salt, Form III has an XRPD pattern comprising 2θ-reflections (+/−0.2 degrees 2θ) at 5.2°, 13.2°, and 13.7°. In some embodiments, Formula I, potassium salt, Form III has an XRPD pattern comprising 2θ-reflections (+/−0.2 degrees 2θ) at 5.2°, 13.2°, and 13.7°, and one, two, or three of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 13.2°, and 13.7°, and one or two of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 13.2°, and 13.7°, and one of the degree 2θ-reflections (+/−0.2 degrees 2θ) at 11.2°, 12.4°, and 18.7°. In some embodiments, Formula I, potassium salt, Form III has an XRPD pattern comprising degree 2θ-reflections (+/−0.2 degrees 2θ) at 5.2°, 13.2°, and 13.7°, and two of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°. In some embodiments, Formula I, potassium salt, Form III has an XRPD pattern comprising any three of the 2θ-reflections (+/−0.2 degrees 2θ) at 5.2°, 11.2°, 12.4°, 13.2°, 13.7°, and 18.7°.

In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°, and one or two of the 2θ-reflections (+/−0.2 degrees 2θ) at 6.5° and 15.0°. In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°, and one of the 2θ-reflections (+/−0.2 degrees 2θ) at 6.5° and 15.0°. In some embodiments, Formula I, potassium salt, Form III has an XRPD pattern comprising 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising any three of the 2θ-reflections (+/−0.2 degrees 2θ) at 5.2°, 6.5°, 11.2°, 12.4°, 13.2°, 13.7°, 15.0°, and 18.7°.

In some embodiments, Formula I, potassium salt, Form III has an XRPD pattern comprising at least three of the 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising at least four of the 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising at least five of the 2θ-reflections (+/−0.2 degrees 2θ) at 5.2°, 6.5°, 11.2°, 12.4°, 13.2°, 13.7°, 15.0°, and 18.7°.

In some embodiments, Formula I, potassium salt, Form III has an XRPD pattern comprising peaks at:

| Pos. | Rel. Int. |
|---|---|
| [°2Th.] | [%] |
| 5.2 | 100 |
| 6.5 | 13 |
| 11.2 | 27 |
| 12.4 | 25 |
| 13.2 | 31 |
| 13.7 | 58 |
| 15.0 | 14 |
| 18.7 | 22 |

### Diethylamine Salt of the Compound of Formula I

In some embodiments, the composition comprises a diethylamine salt of the compound of Formula I which can be amorphous or crystalline. In some embodiments, the diethylamine salt of the compound of Formula I is crystalline.

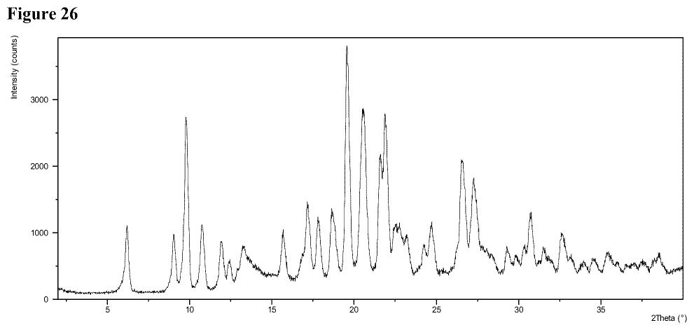

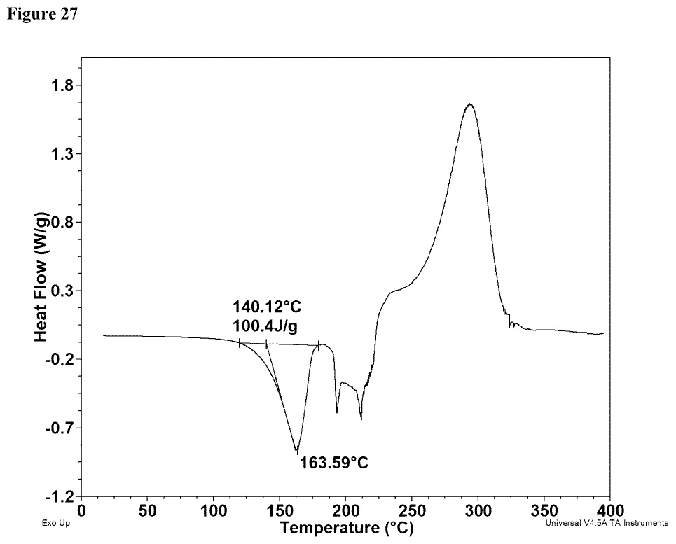

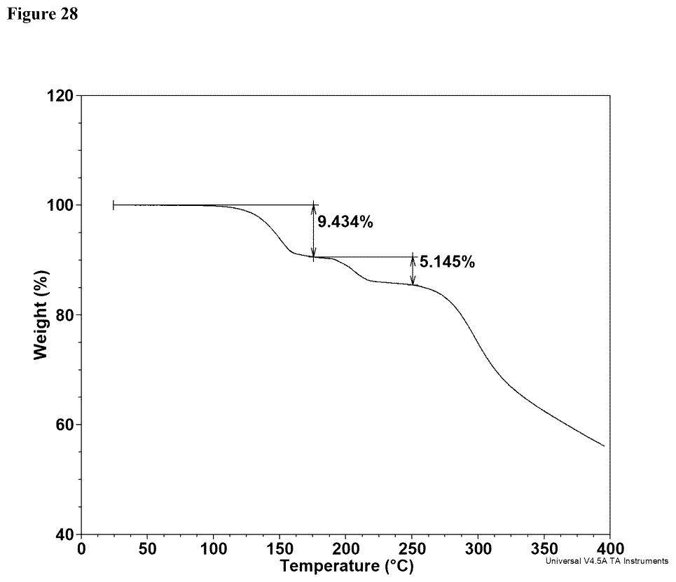

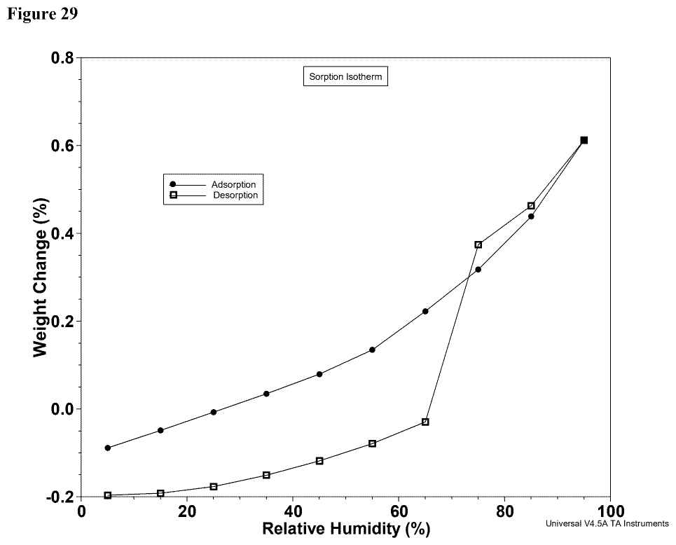

In some embodiments, the crystalline diethylamine salt of the compound of Formula I has an XRPD profile substantially as shown in FIG. 26. The crystalline diethylamine salt of the compound of Formula I may exhibit a DSC thermogram substantially as shown in FIG. 27. The crystalline diethylamine salt of the compound of Formula I may exhibit a TGA graph substantially as shown in FIG. 28. The crystalline diethylamine salt of the compound of Formula I may exhibit a DVS curve substantially as shown in FIG. 29.

In some embodiments, the crystalline diethylamine salt of the compound of Formula I has an XRPD pattern displaying at least two, at least three, at least four, at least five, or at least six of the degree 2θ-reflections with the greatest intensity as the XRPD pattern substantially as shown in FIG. 26.

In some embodiments, the crystalline diethylamine salt of the compound of Formula I has an XRPD pattern comprising 2θ-reflections (+/−0.2 degrees 2θ) at 9.7°, 19.5° and 20.5°. In some embodiments, the crystalline diethylamine salt of the compound of Formula I has an XRPD pattern comprising 2θ-reflections (+/−0.2 degrees 2θ) at 9.7°, 19.5° and 20.5°, and one, two or three of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and one or two of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and one of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and two of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°. In some embodiments, the crystalline diethylamine salt of the compound of Formula I has an XRPD pattern comprising any three of the 2θ-reflections (+/−0.2 degrees 2θ) at 9.7°, 19.5°, 20.5°, 21.9°, 26.5°, and 27.2°.

In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one, two or three of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one or two of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one of the degree 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and two of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 6.2°, 9.7°, 10.7°, 17.1°, 19.5°, 20.5°, 21.9°, 26.5°, and 27.2°. In some embodiments, the crystalline diethylamine salt of the compound of Formula I has an XRPD pattern comprising any three of the 2θ-reflections (+/−0.2 degrees 2θ) at 6.2°, 9.7°, 10.7°, 17.1°, 19.5°, 20.5°, 21.9°, 26.5°, and 27.2°.

In some embodiments, the crystalline diethylamine salt of the compound of Formula I has an XRPD pattern comprising at least three of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°. In some embodiments, the crystalline diethylamine salt of the compound of Formula I has an XRPD pattern comprising at least four of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°. In some embodiments, the crystalline diethylamine salt of the compound of Formula I has an XRPD pattern comprising at least five of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°.

In some embodiments, the crystalline diethylamine salt of the compound of Formula I has an XRPD pattern comprising peaks at:

| Pos. | Rel. Int. |
|---|---|
| [°2Th.] | [%] |
| 6.2 | 29 |
| 9.0 | 24 |
| 9.7 | 72 |
| 10.7 | 28 |
| 11.9 | 20 |
| 12.4 | 9 |
| 13.2 | 13 |
| 15.7 | 19 |
| 17.1 | 32 |
| 17.8 | 25 |
| 18.6 | 26 |
| 19.5 | 100 |
| 20.5 | 69 |
| 21.5 | 49 |
| 21.9 | 68 |
| 23.2 | 15 |
| 24.7 | 21 |
| 26.5 | 48 |
| 27.2 | 38 |
| 30.7 | 23 |
| 32.5 | 13 |
| 35.4 | 7 |

In some embodiments, the crystalline diethylamine salt of the compound of Formula I is characterized by a DSC thermogram substantially as shown in FIG. 27. In some embodiments, the crystalline diethylamine salt of the compound of Formula I is characterized by a DSC thermogram having an endothermic transition at about 140° C.

In some embodiments, the crystalline diethylamine salt of the compound of Formula I is characterized by a TGA curve substantially as shown in FIG. 28. In some embodiments, the crystalline diethylamine salt of the compound of Formula I loses mass in multiple stages with one stage starting at about 100° C. and the next stage starting at about 175° C.

In some embodiments, the crystalline diethylamine salt of the compound of Formula I is characterized by a DVS curve substantially as shown in shown in FIG. 29. In some embodiments, the crystalline diethylamine salt of the compound of Formula I absorbs less than about 1% of water up to 95% RH at 25° C.

### Ammonia Salt of the Compound of Formula I

In some embodiments, pharmaceutical composition comprises an ammonia salt of the compound of Formula I which can be amorphous or crystalline. In some embodiments, the ammonia salt of the compound of Formula I is crystalline.

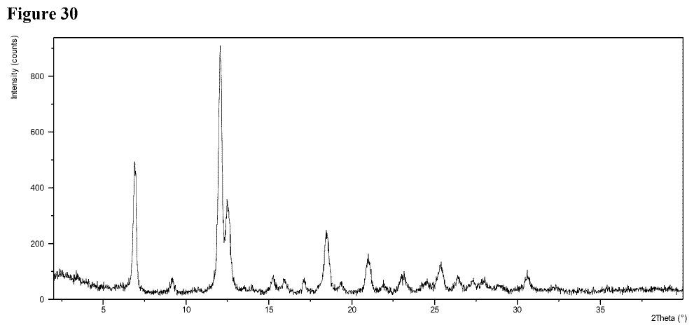

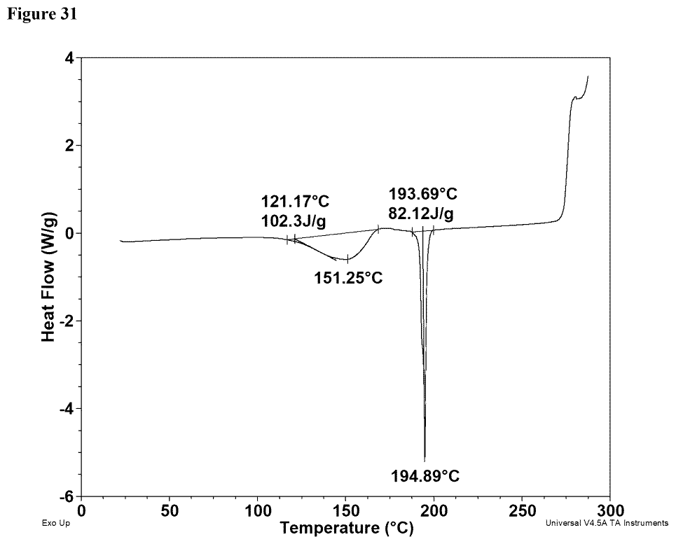

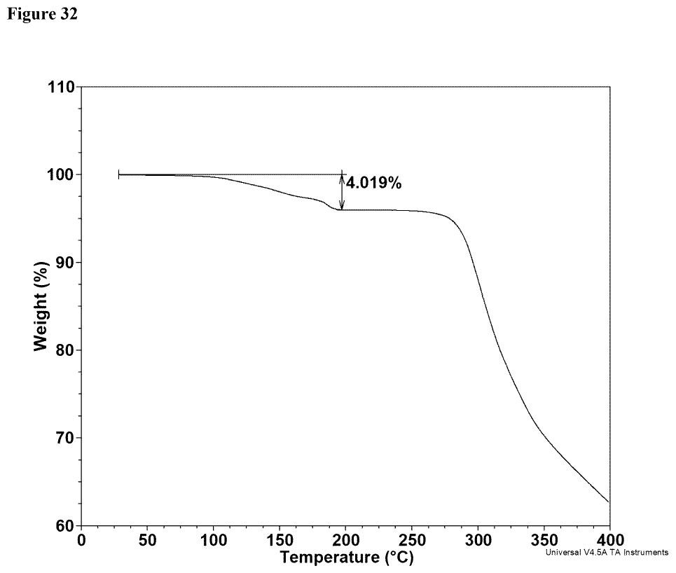

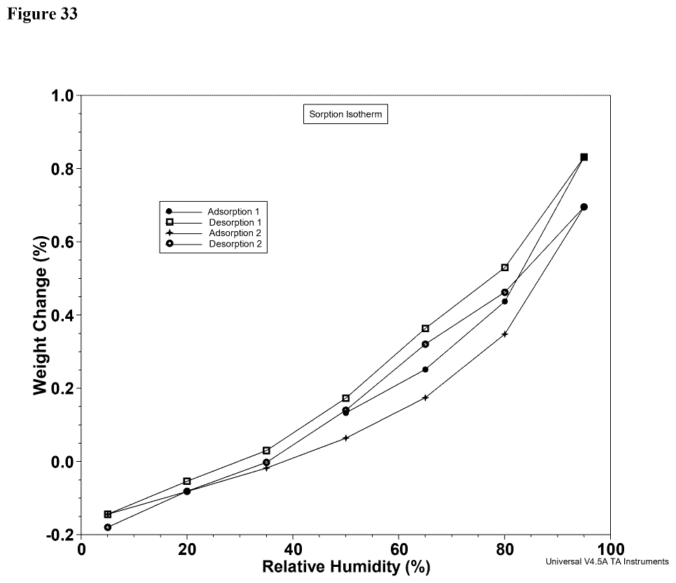

In some embodiments, the crystalline ammonia salt of the compound of Formula I has an XRPD profile substantially as shown in FIG. 30. The crystalline ammonia salt of the compound of Formula I may exhibit a DSC thermogram substantially as shown in FIG. 31. The crystalline ammonia salt of the compound of Formula I may exhibit a TGA graph substantially as shown in FIG. 32. The crystalline ammonia salt of the compound of Formula I may exhibit a DVS curve substantially as shown in FIG. 33.

In some embodiments, the crystalline ammonia salt of the compound of Formula I has an XRPD pattern displaying at least two, at least three, at least four, at least five, or at least six of the degree 2θ-reflections with the greatest intensity as the XRPD pattern substantially as shown in FIG. 30.

In some embodiments, the crystalline ammonia salt of the compound of Formula I has an XRPD pattern comprising 2θ-reflections (+/−0.2 degrees 2θ) at 6.9°, 12.1°, and 12.5°. In some embodiments, the crystalline ammonia salt of the compound of Formula I has an XRPD pattern comprising 2θ-reflections (+/−0.2 degrees 2θ) at 6.9°, 12.1°, and 12.5°, and one, two or three of the 2θ-reflections (+/−0.2 degrees 2θ) at 18.4°, 21.0°, and 25.4°. In some embodiments, the crystalline ammonia salt of the compound of Formula I has an XRPD pattern comprising 20-reflections (+/−0.2 degrees 2θ) at 6.9°, 12.1°, and 12.5°, and one or two of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, and 12.5°, and one of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, and 12.5°, and two of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°. In some embodiments, the crystalline ammonia salt of the compound of Formula I has an XRPD pattern comprising any three of the 2θ-reflections (+/−0.2 degrees 2θ) at 6.9°, 12.1°, 12.5°, 18.4°, 21.0°, and 25.4°.

In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one, two or three of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one or two of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and two of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 9.2°, 12.1°, 12.5°, 18.4°, 21.0°, 23.1°, 25.4°, and 30.6°. In some embodiments, the crystalline ammonia salt of the compound of Formula I has an XRPD pattern comprising any three of the 2θ-reflections (+/−0.2 degrees 2θ) at 6.9°, 9.2°, 12.1°, 12.5°, 18.4°, 21.0°, 23.1°, 25.4°, and 30.6°.

In some embodiments, the crystalline ammonia salt of the compound of Formula I has an XRPD pattern comprising at least three of the 2θ-reflections (+/−0.2 degrees 2θ) at 6.9°, 9.2°, 12.1°, 12.5°, 15.6°, 17.1°, 18.4°, 21.0°, 23.1°, 25.4°, 26.4°, and 30.6°. In some embodiments, the crystalline ammonia salt of the compound of Formula I has an XRPD pattern comprising at least four of the 2θ-reflections (+/−0.2 degrees 2θ) at 6.9°, 9.2°, 12.1°, 12.5°, 15.6°, 17.1°, 18.4°, 21.0°, 23.1°, 25.4°, 26.4°, and 30.6°. In some embodiments, the crystalline ammonia salt of the compound of Formula I has an XRPD pattern comprising at least five of the 2θ-reflections (+/−0.2 degrees 2θ) at 6.9°, 9.2°, 12.1°, 12.5°, 15.6°, 17.1°, 18.4°, 21.0°, 23.1°, 25.4°, 26.4°, and 30.6°.

In some embodiments, the crystalline ammonia salt of the compound of Formula I has an XRPD pattern comprising peaks at:

| Pos. | Rel. Int. |
|---|---|
| [°2Th.] | [%] |
| 6.9 | 56 |
| 9.2 | 6 |
| 12.1 | 100 |
| 12.5 | 38 |
| 15.6 | 2 |
| 17.1 | 4 |
| 18.4 | 25 |
| 21.0 | 15 |
| 23.1 | 6 |
| 25.4 | 11 |
| 26.4 | 5 |
| 30.6 | 6 |

In some embodiments, the crystalline ammonia salt of the compound of Formula I is characterized by a DSC thermogram substantially as shown in FIG. 31. In some embodiments, the crystalline ammonia salt of the compound of Formula I is characterized by a DSC thermogram having one or both of (i) endothermic transition at about 121° C. and (ii) an endothermic transition at about 194° C. In some embodiments, the crystalline ammonia salt of the compound of Formula I is characterized by a DSC thermogram having (i) endothermic transition at about 121° C. and (ii) an endothermic transition at about 194° C.

In some embodiments, the crystalline ammonia salt of the compound of Formula I is characterized by a TGA curve substantially as shown in FIG. 32. In some embodiments, the crystalline ammonia salt of the compound of Formula I loses mass starting at about 80° C.

In some embodiments, the crystalline ammonia salt of the compound of Formula I is characterized by a DVS curve substantially as shown in shown in FIG. 33. In some embodiments, the crystalline ammonia salt of the compound of Formula I absorbs less than about 1% of water up to 95% RH at 25° C.

### Calcium Salt of the Compound of Formula I

In some embodiments, the pharmaceutical composition comprises a calcium salt of the compound of Formula I which can be in any form such as, for example, crystalline or amorphous. In some embodiments, the calcium salt of the compound of Formula I is crystalline.

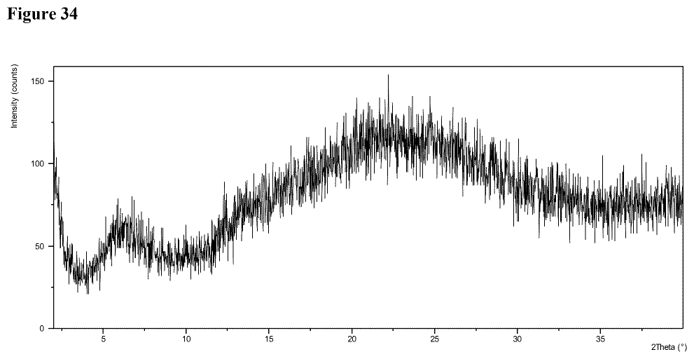

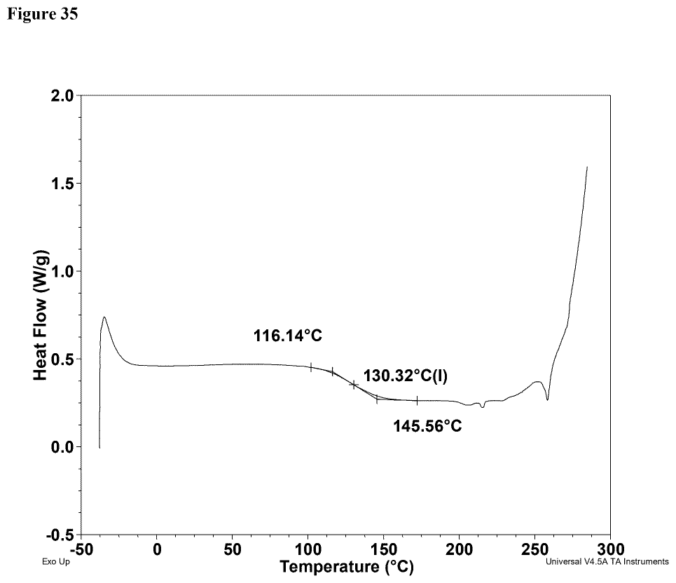

In some embodiments, the crystalline calcium salt of the compound of Formula I is a Form I (“compound of Formula I, calcium salt, Form I” or “Formula I, calcium salt, Form I”) having an XRPD profile substantially as shown in FIG. 34. Compound of Formula I, calcium salt, Form I may exhibit a DSC thermogram substantially as shown in FIG. 35.

In some embodiments, the Compound of Formula I, calcium salt, Form I is characterized by a DSC thermogram substantially as shown in FIG. 35. In some embodiments, the Compound of Formula I, calcium salt, Form I is characterized by a DSC thermogram having a glass transition at about 130° C.

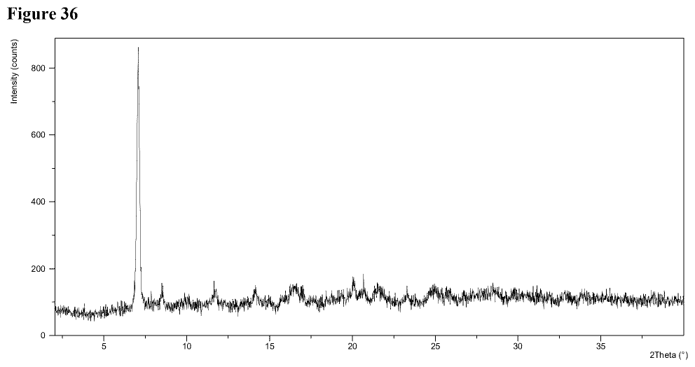

In some embodiments, the crystalline calcium salt of the compound of Formula I is a Form II (“compound of Formula I, calcium salt, Form II” or “Formula I, calcium salt, Form II”). In some embodiments, the compound of Formula I, calcium salt, Form II has an XRPD profile substantially as shown in FIG. 36.

In some embodiments, Formula I, calcium salt, Form II has an XRPD pattern displaying at least two, at least three, at least four, at least five, or at least six of the degree 2θ-reflections with the greatest intensity as the XRPD pattern substantially as shown in FIG. 36.

In some embodiments, the Formula I, calcium salt, Form II has an XRPD pattern comprising 2θ-reflections (+/−0.2 degrees 2θ) at 7.1°, 8.5°, and 11.7°. In some embodiments, the Formula I, calcium salt, Form II has an XRPD pattern comprising 2θ-reflections (+/−0.2 degrees 2θ) at 7.1°, 8.5°, and 11.7°, and one, two or three of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and one or two of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and one of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and two of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, 11.7°, 14.1°, 16.5°, and 20.1°. In some embodiments, the Formula I, calcium salt, Form II has an XRPD pattern comprising any three of the 2θ-reflections (+/−0.2 degrees 2θ) at 7.1°, 8.5°, 11.7°, 14.1°, 16.5°, and 20.1°.

In some embodiments, the Formula I, calcium salt, Form II has an XRPD pattern comprising at least three of the 2θ-reflections (+/−0.2 degrees 2θ) at 7.1°, 8.5°, 11.7°, 14.1°, 16.5°, and 20.1°. In some embodiments, the Formula I, calcium salt, Form II has an XRPD pattern comprising at least four of the 2θ-reflections (+/−0.2 degrees 2θ) at 7.1°, 8.5°, 11.7°, 14.1°, 16.5°, and 20.1°. In some embodiments, the Formula I, calcium salt, Form II has an XRPD pattern comprising at least five of the 2θ-reflections (+/−0.2 degrees 2θ) at 7.1°, 8.5°, 11.7°, 14.1°, 16.5°, and 20.1°.

In some embodiments, the Formula I, calcium salt, Form II has an XRPD pattern comprising peaks at:

| Pos. | Rel. Int. |
|---|---|
| [°2Th.] | [%] |
| 7.1 | 100 |
| 8.5 | 7 |
| 11.7 | 5 |
| 14.1 | 6 |
| 16.5 | 7 |
| 20.1 | 7 |
| 21.5 | 5 |
| 24.8 | 5 |

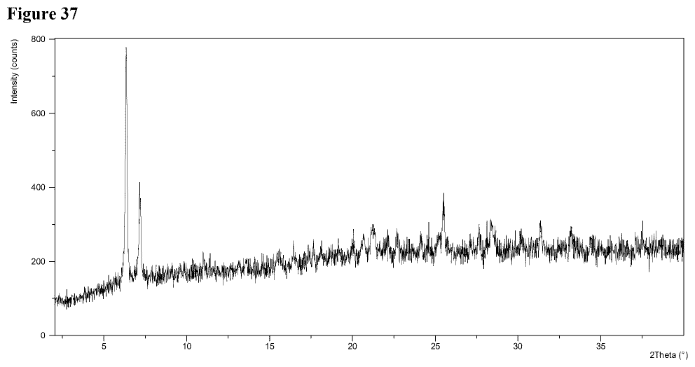

In some embodiments, the crystalline calcium salt of the compound of Formula I is a Form III (“compound of Formula I, calcium salt, Form III” or “Formula I, calcium salt, Form III”). In some embodiments, the compound of Formula I, calcium salt, Form III has an XRPD profile substantially as shown in FIG. 37.

In some embodiments, Formula I, calcium salt, Form III has an XRPD pattern displaying at least two, at least three, at least four, at least five, or at least six of the degree 2θ-reflections with the greatest intensity as the XRPD pattern substantially as shown in FIG. 37.

In some embodiments, the Formula I, calcium salt, Form III has an XRPD pattern comprising 2θ-reflections (+/−0.2 degrees 2θ) at 6.3°, 7.2°, and 25.5°. In some embodiments, the Formula I, calcium salt, Form III has an XRPD pattern comprising 2θ-reflections (+/−0.2 degrees 2θ) at 6.3°, 7.2°, and 25.5°, and one, two or three of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and one or two of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and one of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and two of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising any three of the 2θ-reflections (+/−0.2 degrees 2θ) at 6.3°, 7.2°, 21.2°, 25.5°, 28.4°, and 31.4°.

In some embodiments, the Formula I, calcium salt, Form III has an XRPD pattern comprising at least three of the 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising at least four of the 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising at least five of the 2θ-reflections (+/−0.2 degrees 2θ) at 6.3°, 7.2°, 21.2°, 25.5°, 28.4°, and 31.4°.

In some embodiments, the Formula I, calcium salt, Form III has an XRPD pattern comprising peaks at:

| Pos. | Rel. Int. |
|---|---|
| [°2Th.] | [%] |
| 6.3 | 100 |
| 7.2 | 35 |
| 21.2 | 11 |
| 25.5 | 22 |
| 28.4 | 11 |
| 31.4 | 9 |
| 6.3 | 100 |
| 7.2 | 35 |

### Magnesium Salt of the Compound of Formula I

In some embodiments, the pharmaceutical composition comprises a magnesium salt of the compound of Formula I which can be in any form such as, for example, crystalline or amorphous. In some embodiments, the magnesium salt of the compound of Formula I is crystalline.

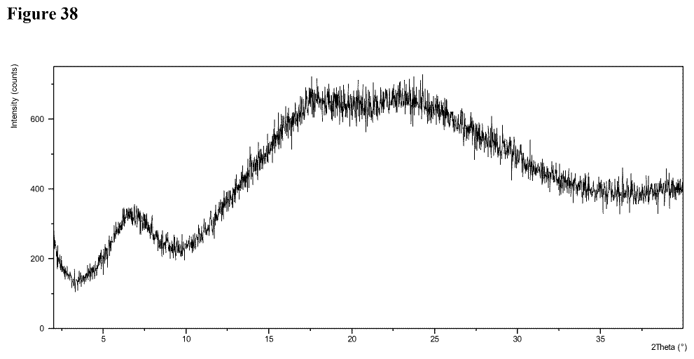

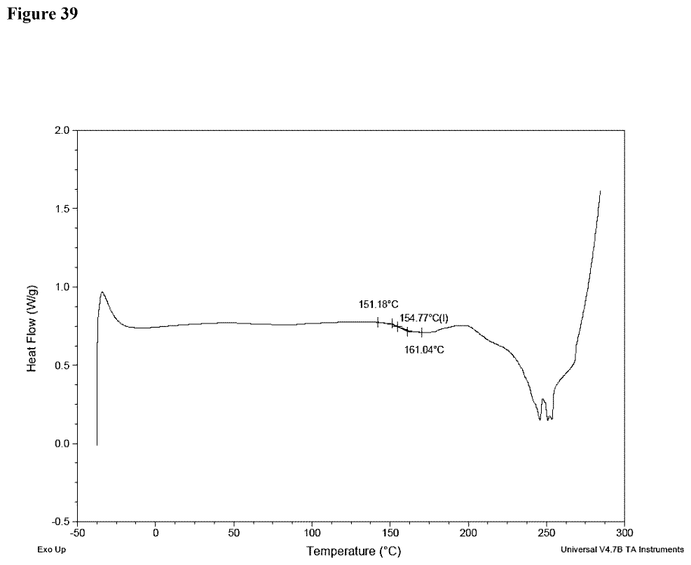

In some embodiments, the crystalline magnesium salt of the compound of Formula I is a Form I (“compound of Formula I, magnesium salt, Form I” or “Formula I, magnesium salt, Form I). In some embodiments, Compound of Formula I, magnesium salt, Form I has an XRPD profile substantially as shown in FIG. 38. The Formula I, magnesium salt, Form I may exhibit a DSC thermogram substantially as shown in FIG. 39.

In some embodiments, Formula I, magnesium salt, Form I is characterized by a DSC thermogram substantially as shown in FIG. 39. In some embodiments, Formula I, magnesium salt, Form I is characterized by a DSC thermogram having a glass transition at about 155° C.

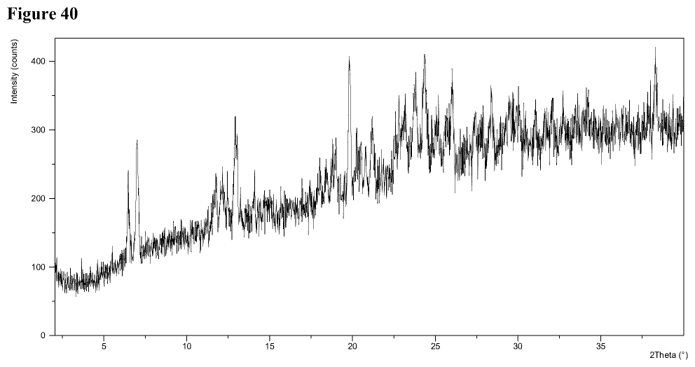

In some embodiments, the crystalline magnesium salt of the compound of Formula I is a Form II (“compound of Formula I, magnesium salt, Form II” or “Formula I, magnesium salt, Form II). In some embodiments, Compound of Formula I, magnesium salt, Form II has an XRPD profile substantially as shown in FIG. 40.

In some embodiments, Formula I, magnesium salt, Form II has an XRPD pattern displaying at least two, at least three, at least four, at least five, or at least six of the degree 2θ-reflections with the greatest intensity as the XRPD pattern substantially as shown in FIG. 40.

In some embodiments, the Formula I, magnesium salt, Form II has an XRPD pattern comprising 2θ-reflections (+/−0.2 degrees 2θ) at 7.0°, 19.8°, and 24.3°. In some embodiments, the Formula I, magnesium salt, Form II has an XRPD pattern comprising 2θ-reflections (+/−0.2 degrees 2θ) at 7.0°, 19.8°, and 24.3°, and one, two or three of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and one or two of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and one of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and two of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising any three of the 2θ-reflections (+/−0.2 degrees 2θ) at 6.5°, 7.0°, 13.0°, 19.8°, 21.2°, and 24.3°.

In some embodiments, the Formula I, magnesium salt, Form II has an XRPD pattern comprising at least three of the 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising at least four of the 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising at least five of the 2θ-reflections (+/−0.2 degrees 2θ) at 6.5°, 7.0°, 13.0°, 19.8°, 21.2°, and 24.3°.

In some embodiments, the Formula I, magnesium salt, Form II has an XRPD pattern comprising peaks at:

| Pos. | Rel. Int. |
|---|---|
| [°2Th.] | [%] |
| 6.5 | 51 |
| 7.0 | 88 |
| 13.0 | 65 |
| 19.8 | 100 |
| 21.2 | 44 |
| 24.3 | 77 |
| 25.9 | 38 |

### N-Butylamine Salt of the Compound of Formula I

In some embodiments, the pharmaceutical composition comprises a N-butylamine salt of the compound of Formula I which can be amorphous or crystalline. In some embodiments, the N-butylamine salt of the compound of Formula I is crystalline.

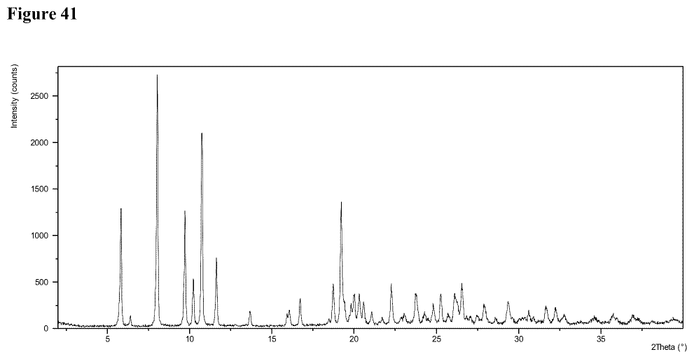

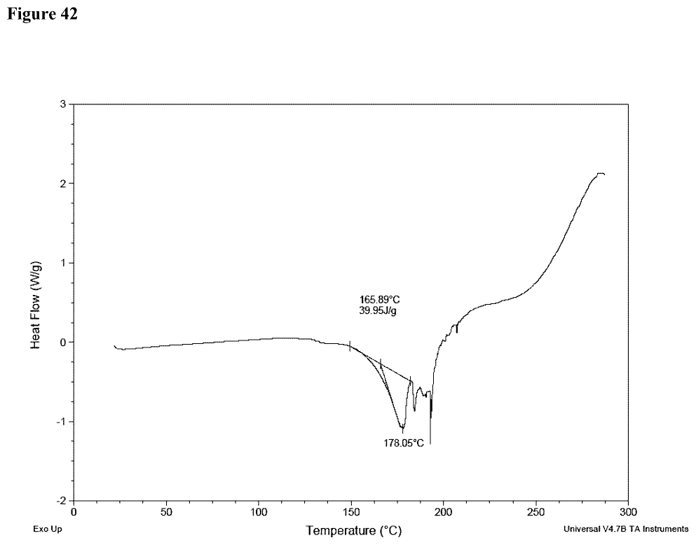

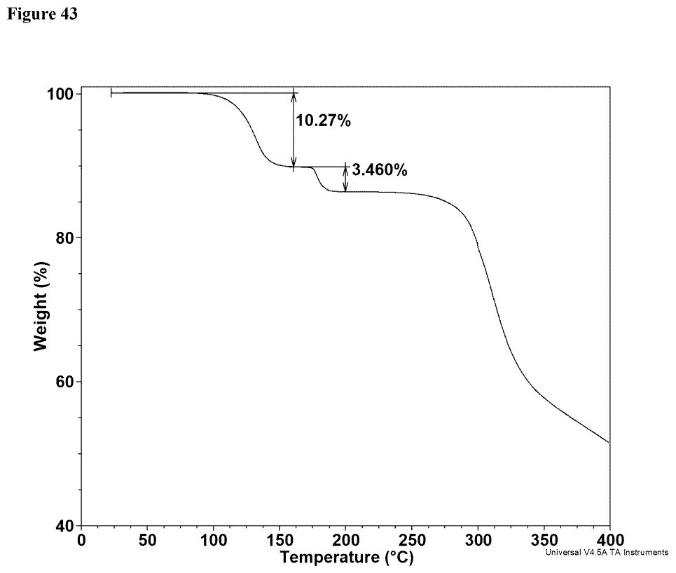

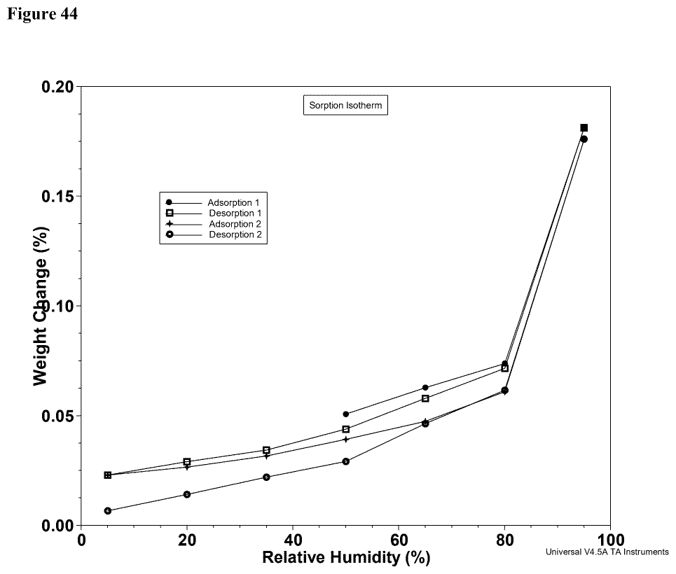

In some embodiments, the crystalline N-butylamine salt of the compound of Formula I has an XRPD profile substantially as shown in FIG. 41. The crystalline N-butylamine salt of the compound of Formula I may exhibit a DSC thermogram substantially as shown in FIG. 42. The crystalline N-butylamine salt of the compound of Formula I may exhibit a TGA graph substantially as shown in FIG. 43. The crystalline N-butylamine salt of the compound of Formula I may exhibit a DVS curve substantially as shown in FIG. 44.

In some embodiments, the crystalline N-butylamine salt of the compound of Formula I has an XRPD pattern comprising 2θ-reflections (+/−0.2 degrees 2θ) at 5.8°, 8.0°, and 10.7°. In some embodiments, the crystalline N-butylamine salt of the compound of Formula I has an XRPD pattern comprising 2θ-reflections (+/−0.2 degrees 2θ) at 5.8°, 8.0°, and 10.7°, and one, two or three of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and one or two of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and one of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and two of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°. In some embodiments, the crystalline N-butylamine salt of the compound of Formula I has an XRPD pattern comprising any three of the 2θ-reflections (+/−0.2 degrees 2θ) at 5.8°, 8.0°, 9.7°, 10.7°, 11.6°, and 19.2°

In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one, two or three of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one or two of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and two of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, 18.7°, 19.2°, 22.3°, and 26.5°. In some embodiments, the crystalline N-butylamine salt of the compound of Formula I has an XRPD pattern comprising any three of the 2θ-reflections (+/−0.2 degrees 2θ) at 5.8°, 8.0°, 9.7°, 10.7°, 11.6°, 18.7°, 19.2°, 22.3°, and 26.5°.

In some embodiments, the crystalline N-butylamine salt of the compound of Formula I has an XRPD pattern comprising at least three of the 2θ-reflections (+/−0.2 degrees 2θ) at 5.8°, 8.0°, 9.7°, 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising at least four of the 2θ-reflections (+/−0.2 degrees 2θ) at 5.8°, 8.0°, 9.7°, 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising at least five of the 2θ-reflections (+/−0.2 degrees 2θ) at 5.8°, 8.0°, 9.7°, 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising peaks at:

| Pos. | Rel. Int. |
|---|---|
| [°2Th.] | [%] |
| 5.8 | 48 |
| 8.0 | 100 |
| 9.7 | 45 |
| 10.2 | 17 |
| 10.7 | 78 |
| 11.6 | 26 |
| 16.0 | 5 |
| 16.7 | 10 |
| 18.7 | 16 |
| 19.2 | 45 |
| 20.3 | 11 |
| 22.3 | 13 |
| 23.0 | 3 |
| 23.7 | 11 |
| 25.3 | 10 |
| 26.1 | 10 |
| 26.5 | 14 |
| 27.9 | 7 |
| 31.6 | 6 |

In some embodiments, the crystalline N-butylamine salt of the compound of Formula I is characterized by a DSC thermogram substantially as shown in FIG. 42.

In some embodiments, the crystalline N-butylamine salt of the compound of Formula I is characterized by a DSC thermogram having an endothermic transition with an onset at about 166° C.

In some embodiments, the crystalline N-butylamine salt of the compound of Formula I is characterized by a TGA curve substantially as shown in FIG. 43. In some embodiments, the crystalline N-butylamine salt of the compound of Formula I loses mass in multiples stages starting at about 80° C. and another stage starting at about 175° C.

In some embodiments, the crystalline N-butylamine salt of the compound of Formula I is characterized by a DVS curve substantially as shown in shown in FIG. 44. In some embodiments, the crystalline N-butylamine salt of the compound of Formula I absorbs less than about 0.2% of water up to 95% RH at 25° C.

### Diethanolamine Salt of the Compound of Formula I

In some embodiments, the pharmaceutical composition comprises a diethanolamine salt of the compound of Formula I which can be amorphous or crystalline. In some embodiments, the diethanolamine salt of the compound of Formula I is crystalline.

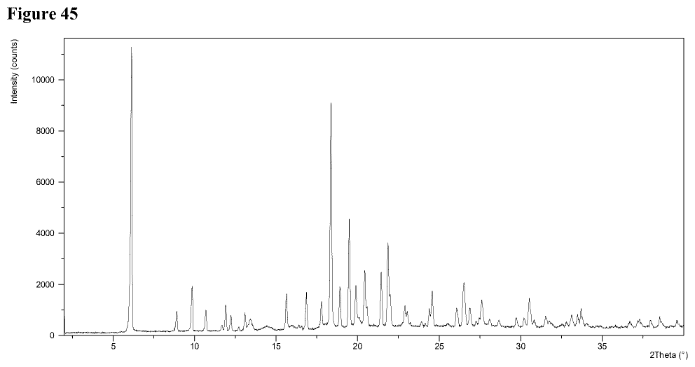

In some embodiments, the crystalline diethanolamine salt of the compound of Formula I has an XRPD profile substantially as shown in FIG. 45. In some embodiments, the crystalline diethanolamine salt of the compound of Formula I has an XRPD pattern displaying at least two, at least three, at least four, at least five, or at least six of the degree 2θ-reflections with the greatest intensity as the XRPD pattern substantially as shown in FIG. 45.

In some embodiments, the crystalline diethanolamine salt of the compound of Formula I has an XRPD pattern comprising 2θ-reflections (+/−0.2 degrees 2θ) at 6.1°, 18.4°, and 19.5°. In some embodiments, the crystalline diethanolamine salt of the compound of Formula I has an XRPD pattern comprising 2θ-reflections (+/−0.2 degrees 2θ) at 6.1°, 18.4°, and 19.5°, and one, two or three of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and one or two of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and one of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and two of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°. In some embodiments, the crystalline diethanolamine salt of the compound of Formula I has an XRPD pattern comprising any three of the 2θ-reflections (+/−0.2 degrees 2θ) at 6.1°, 9.9°, 18.4°, 19.5°, 20.4°, and 21.8°.

In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one, two or three of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one or two of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and two of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising any three of the 2θ-reflections (+/−0.2 degrees 2θ) at 6.1°, 9.9°, 16.9°, 18.4°, 18.9°, 19.5°, 20.4°, 21.8°, and 26.5°.

In some embodiments, the crystalline diethanolamine salt of the compound of Formula I has an XRPD pattern comprising at least three of the 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising at least four of the 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising at least five of the 2θ-reflections (+/−0.2 degrees 2θ) at 6.1°, 9.9°, 16.9°, 18.4°, 18.9°, 19.5°, 20.4°, 21.8°, and 26.5°.

In some embodiments, the crystalline diethanolamine salt of the compound of Formula I has an XRPD pattern comprising peaks at:

| Pos. | Rel. Int. |
|---|---|
| [°2Th.] | [%] |
| 6.1 | 100 |
| 9.9 | 16 |
| 11.9 | 9 |
| 13.2 | 1 |
| 15.6 | 12 |
| 16.9 | 13 |
| 18.4 | 79 |
| 18.9 | 14 |
| 19.5 | 38 |
| 20.4 | 20 |
| 21.4 | 19 |
| 21.8 | 29 |
| 22.9 | 5 |
| 24.6 | 11 |
| 26.5 | 15 |
| 30.5 | 10 |
| 33.5 | 2 |

### Ethylenediamine Salt of the Compound of Formula I

In some embodiments, the pharmaceutical composition comprises an ethylenediamine salt of the compound of Formula I which can be amorphous or crystalline. In some embodiments, the ethylenediamine salt of the compound of Formula I is crystalline.

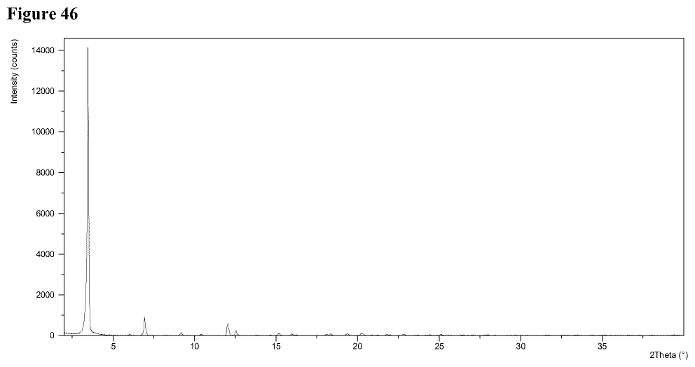

In some embodiments, the crystalline ethylenediamine salt of the compound of Formula I has an XRPD profile substantially as shown in FIG. 46. In some embodiments, the crystalline ethylenediamine salt of the compound of Formula I has an XRPD pattern displaying at least two, at least three, at least four, at least five, or at least six of the degree 2θ-reflections with the greatest intensity as the XRPD pattern substantially as shown in FIG. 46.

In some embodiments, the crystalline ethylenediamine salt of the compound of Formula I has an XRPD pattern comprising 2θ-reflections (+/−0.2 degrees 2θ) at 3.5°, 6.9°, and 12.0°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one, two or three of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one or two of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and two of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising any three of the 2θ-reflections (+/−0.2 degrees 2θ) at 3.5°, 6.9°, 9.2°, 12.0°, 12.5°, and 20.2°.

In some embodiments, the crystalline ethylenediamine salt of the compound of Formula I has an XRPD pattern comprising at least three of the 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising at least four of the 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising at least five of the 2θ-reflections (+/−0.2 degrees 2θ) at 3.5°, 6.9°, 9.2°, 12.0°, 12.5°, and 20.2°.

In some embodiments, the crystalline ethylenediamine salt of the compound of Formula I has an XRPD pattern comprising peaks at:

| Pos. | Rel. Int. |
|---|---|
| [°2Th.] | [%] |
| 3.5 | 100 |
| 6.9 | 6 |
| 9.2 | 1 |
| 12.0 | 4 |
| 12.5 | 1 |
| 15.2 | 1 |
| 19.4 | 1 |
| 20.2 | 1 |

### Morpholine Salt of the Compound of Formula I

In some embodiments, the pharmaceutical composition comprises a morpholine salt of the compound of Formula I which can be amorphous or crystalline. In some embodiments, the morpholine salt of the compound of Formula I is crystalline.

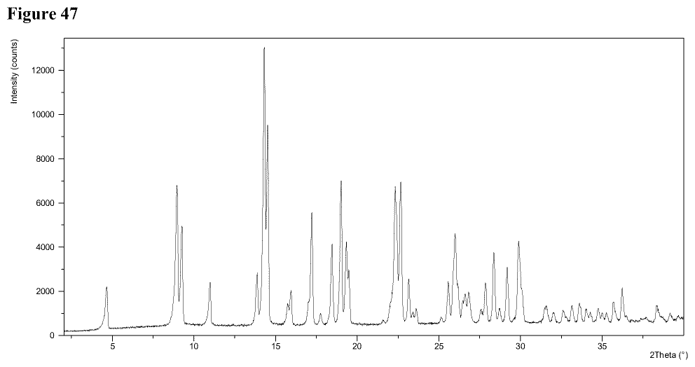

In some embodiments, the crystalline morpholine salt of the compound of Formula I has an XRPD profile substantially as shown in FIG. 47. In some embodiments, the crystalline morpholine salt of the compound of Formula I has an XRPD pattern displaying at least two, at least three, at least four, at least five, or at least six of the degree 2θ-reflections with the greatest intensity as the XRPD pattern substantially as shown in FIG. 47.

In some embodiments, the crystalline morpholine salt of the compound of Formula I has an XRPD pattern comprising 2θ-reflections (+/−0.2 degrees 2θ) at 14.3°, 19.0°, and 22.7°. In some embodiments, the crystalline morpholine salt of the compound of Formula I has an XRPD pattern comprising 2θ-reflections (+/−0.2 degrees 2θ) at 14.3°, 19.0°, and 22.7°, and one, two or three of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and one or two of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and one of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and two of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°. In some embodiments, the crystalline morpholine salt of the compound of Formula I has an XRPD pattern comprising any three of the 2θ-reflections (+/−0.2 degrees 2θ) at 9.0°, 14.3°, 14.5°, 19.0°, 22.3°, and 22.7°.

In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one, two or three of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one or two of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and two of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising any three of the 2θ-reflections (+/−0.2 degrees 2θ) at 4.7°, 9.0°, 14.3°, 14.5°, 17.2°, 19.0°, 22.3°, 22.7°, and 26.0°.

In some embodiments, the crystalline morpholine salt of the compound of Formula I has an XRPD pattern comprising at least three of the 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising at least four of the 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising at least five of the 2θ-reflections (+/−0.2 degrees 2θ) at 4.7°, 9.0°, 14.3°, 14.5°, 17.2°, 19.0°, 22.3°, 22.7°, and 26.0°.

In some embodiments, the crystalline morpholine salt of the compound of Formula I has an XRPD pattern comprising peaks at:

| Pos. | Rel. Int. |
|---|---|
| [°2Th.] | [%] |
| 4.7 | 15 |
| 9.0 | 47 |
| 9.3 | 32 |
| 11.0 | 15 |
| 13.9 | 18 |
| 14.3 | 100 |
| 14.5 | 69 |
| 17.2 | 40 |
| 18.5 | 28 |
| 19.0 | 49 |
| 19.3 | 28 |
| 22.3 | 47 |
| 22.7 | 50 |
| 26.0 | 31 |
| 28.4 | 24 |
| 29.2 | 19 |
| 29.9 | 28 |
| 36.2 | 11 |

### L-Arginine Salt of the Compound of Formula I

In some embodiments, the pharmaceutical composition comprises an L-arginine salt which can be amorphous or crystalline. In some embodiments, the L-arginine salt of the compound of Formula I is crystalline.

In some embodiments, the crystalline L-arginine salt of the compound of Formula I is a Form I (“compound of Formula I, L-arginine salt, Form I” or “Formula I, L-arginine, Form I”).

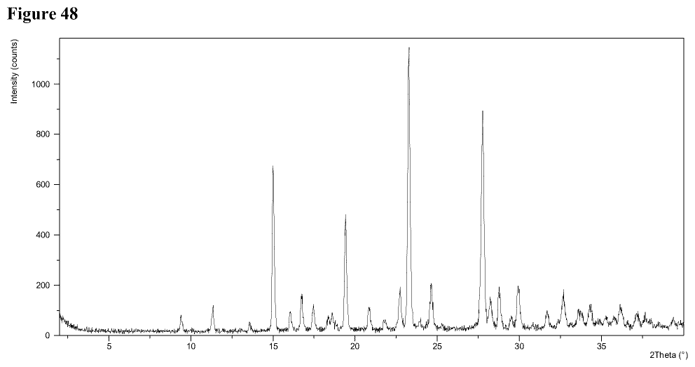

In some embodiments, Formula I, L-arginine salt, Form I has an XRPD profile substantially as shown in FIG. 48. In some embodiments, Formula I, L-arginine salt, Form I has an XRPD pattern displaying at least two, at least three, at least four, at least five, or at least six of the degree 2θ-reflections with the greatest intensity as the XRPD pattern substantially as shown in FIG. 48.

In some embodiments, Formula I, L-arginine salt, Form I has an XRPD pattern comprising 2θ-reflections (+/−0.2 degrees 2θ) at 15.0°, 23.3°, and 27.7°. In some embodiments, Formula I, L-arginine salt, Form I has an XRPD pattern comprising 2θ-reflections (+/−0.2 degrees 2θ) at 15.0°, 23.3°, and 27.7°, and one, two or three of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and one or two of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and one of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and two of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°. In some embodiments, Formula I, L-arginine salt, Form I has an XRPD pattern comprising any three of the 2θ-reflections (+/−0.2 degrees 2θ) at 15.0°, 19.4°, 23.3°, 24.6°, 27.7°, and 29.9°.

In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one, two or three of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one or two of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and two of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising any three of the 2θ-reflections (+/−0.2 degrees 2θ) at 11.3°, 15.0°, 16.7°, 19.4°, 22.7°, 23.3°, 24.6°, 27.7°, and 29.9°.

In some embodiments, Formula I, L-arginine salt, Form I has an XRPD pattern comprising at least three of the 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising at least four of the 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising at least five of the 2θ-reflections (+/−0.2 degrees 2θ) at 11.3°, 15.0°, 16.7°, 19.4°, 22.7°, 23.3°, 24.6°, 27.7°, and 29.9°.

In some embodiments, Formula I, L-arginine salt, Form I has an XRPD pattern

| Pos. | Rel. Int. |
|---|---|
| [°2Th.] | [%] |
| 11.3 | 8 |
| 15.0 | 54 |
| 16.0 | 7 |
| 16.7 | 12 |
| 17.4 | 8 |
| 18.3 | 4 |
| 19.4 | 35 |
| 20.8 | 8 |
| 22.7 | 13 |
| 23.3 | 100 |
| 24.6 | 15 |
| 27.7 | 58 |
| 28.7 | 14 |
| 29.9 | 15 |
| 32.7 | 11 |
| 33.6 | 4 |
| 36.1 | 6 |

In some embodiments, the crystalline L-arginine salt of the compound of Formula I is a Form II (“compound of Formula I, L-arginine salt, Form II” or “Formula I, L-arginine, Form II”).

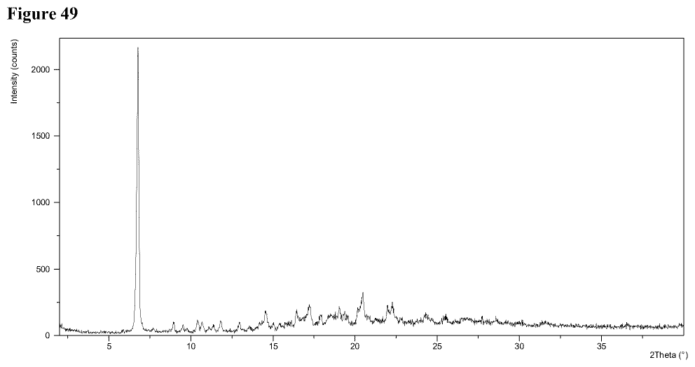

In some embodiments, Formula I, L-arginine salt, Form II has an XRPD profile substantially as shown in FIG. 49. In some embodiments, Formula I, L-arginine salt, Form II has an XRPD pattern displaying at least two, at least three, at least four, at least five, or at least six of the degree 2θ-reflections with the greatest intensity as the XRPD pattern substantially as shown in FIG. 49.

In some embodiments, Formula I, L-arginine salt, Form II has an XRPD pattern comprising 2θ-reflections (+/−0.2 degrees 2θ) at 6.8°, 14.5°, and 20.5°. In some embodiments, Formula I, L-arginine salt, Form II has an XRPD pattern comprising 2θ-reflections (+/−0.2 degrees 2θ) at 6.8°, 14.5°, and 20.5°, and one, two or three of the 2θ-reflections (+/−0.2 degrees 20) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and one or two of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and one of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and two of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising any three of the 2θ-reflections (+/−0.2 degrees 2θ) at 6.8°, 8.9°, 11.8°, 14.5°, 17.2°, and 20.5°.

In some embodiments, Formula I, L-arginine salt, Form II has an XRPD pattern comprising at least three of the 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising at least four of the 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising at least five of the 2θ-reflections (+/−0.2 degrees 2θ) at 6.8°, 8.9°, 11.8°, 14.5°, 17.2°, and 20.5°.

In some embodiments, Formula I, L-arginine salt, Form II has an XRPD pattern

| Pos. | Rel. Int. |
|---|---|
| [°2Th.] | [%] |
| 6.8 | 100 |
| 8.9 | 3 |
| 10.5 | 2 |
| 11.8 | 4 |
| 12.9 | 3 |
| 14.5 | 6 |
| 17.2 | 7 |
| 17.9 | 3 |
| 19.2 | 2 |
| 20.5 | 11 |
| 22.1 | 4 |
| 24.4 | 2 |
| 25.5 | 2 |

In some embodiments, the crystalline L-arginine salt of the compound of Formula I is a Form III (“compound of Formula I, L-arginine salt, Form III” or “Formula I, L-arginine, Form III”).

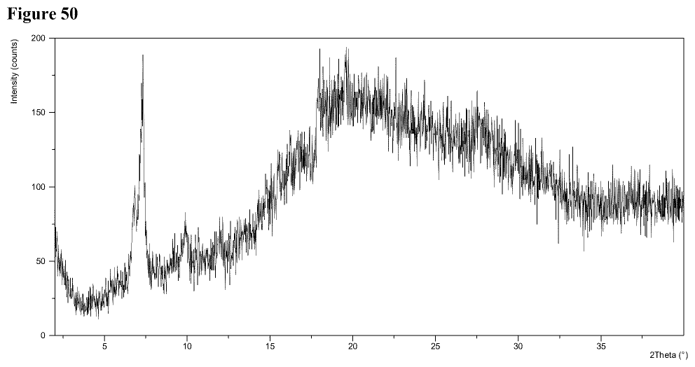

In some embodiments, Formula I, L-arginine salt, Form III has an XRPD profile substantially as shown in FIG. 50. In some embodiments, Formula I, L-arginine salt, Form III has an XRPD pattern displaying at least two, at least three, at least four, at least five, or at least six of the degree 2θ-reflections with the greatest intensity as the XRPD pattern substantially as shown in FIG. 50.

In some embodiments, Formula I, L-arginine salt, Form III has an XRPD pattern comprising a 2θ-reflection (+/−0.2 degrees 2θ) at 7.3°. In some embodiments, Formula I, L-arginine salt, Form III has an XRPD pattern comprising 2θ-reflections (+/−0.2 degrees 2θ) at 7.3° and 9.6°.

In some embodiments, Formula I, L-arginine salt, Form II has an XRPD pattern comprising peaks at:

| Pos. | Rel. Int. |
|---|---|
| [°2Th.] | [%] |
| 7.3 | 100 |
| 9.6 | 10 |

### Trans-Ferulic Acid Salt of the Compound of Formula I

In some embodiments, pharmaceutical composition comprises a trans-Ferulic acid salt of Formula I. In some embodiments, the pharmaceutical comprises a cocrystal of the compound of Formula I. In some embodiments, the trans-ferulic acid salt of the compound of Formula I is a trans-ferulic acid co-crystal (“compound of Formula I trans-ferulic co-crystal” or “Formula I trans-ferulic co-crystal”).

In some embodiments, the compound of Formula I trans-ferulic co-crystal is crystalline.

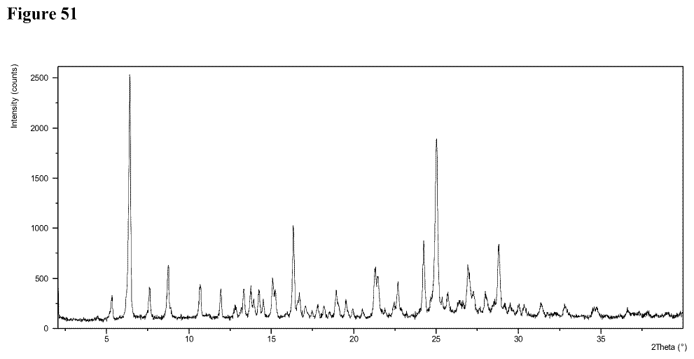

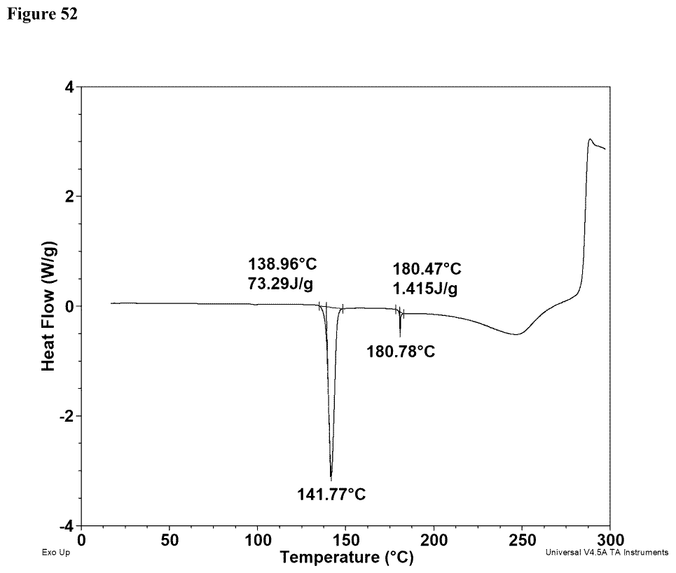

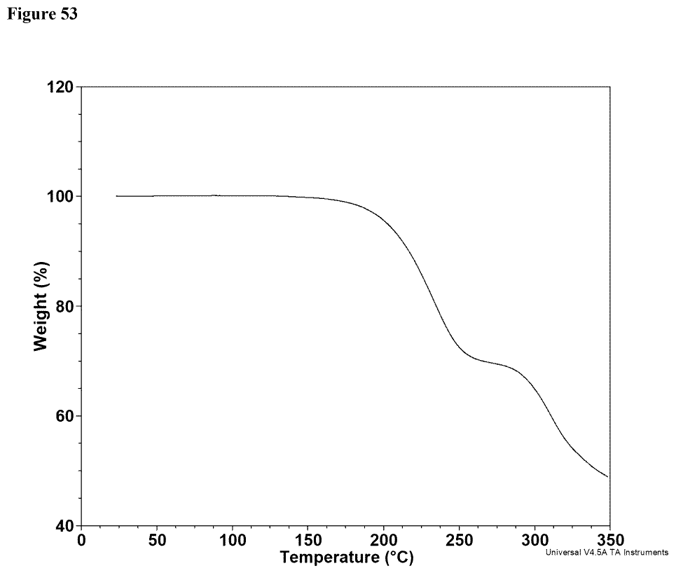

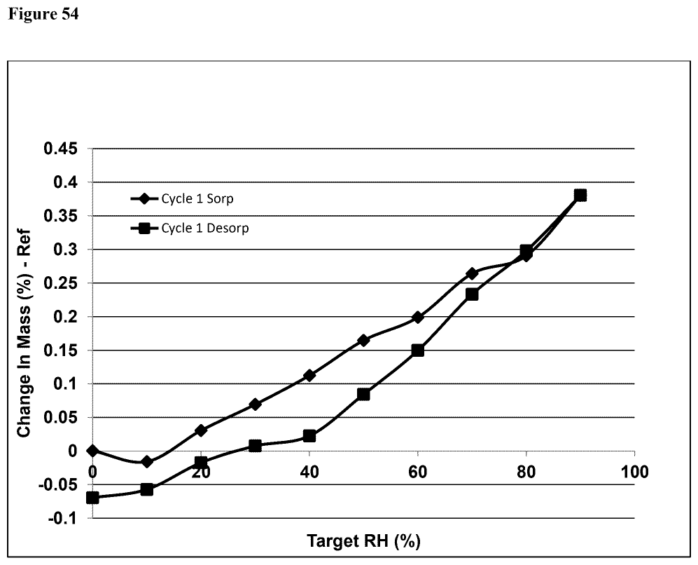

In some embodiments, the crystalline compound of Formula I, trans-ferulic co-crystal is a Form I (“compound of Formula I, trans-ferulic co-crystal, Form I” or “Formula I trans-ferulic co-crystal, Form I”). In some embodiments, compound of Formula I, trans-ferulic co-crystal, Form I has an XRPD profile substantially as shown in FIG. 51. The Formula I trans-ferulic co-crystal, Form I may exhibit a DSC thermogram substantially as shown in FIG. 52. The Formula I trans-ferulic co-crystal, Form I may exhibit a TGA graph substantially as shown in FIG. 53. The Formula I trans-ferulic co-crystal, Form I may exhibit a DVS curve substantially as shown in FIG. 54.

In some embodiments, the Formula I trans-ferulic co-crystal, Form I has an XRPD pattern displaying at least two, at least three, at least four, at least five, or at least six of the degree 2θ-reflections with the greatest intensity as the XRPD pattern substantially as shown in FIG. 51.

In some embodiments, the Formula I trans-ferulic co-crystal, Form I has an XRPD pattern comprising 2θ-reflections (+/−0.2 degrees 2θ) at 6.4°, 16.3°, and 25.0°. In some embodiments, the Formula I trans-ferulic co-crystal, Form I has an XRPD pattern comprising 2θ-reflections (+/−0.2 degrees 2θ) at 6.4°, 16.3°, and 25.0°, and one, two or three of the 2θ-reflections (+/−0.2 degrees 2θ) at 8.7°, 24.2°, and 28.8°. In some embodiments, the Formula I trans-ferulic co-crystal, Form I has an XRPD pattern comprising 2θ-reflections (+/−0.2 degrees 2θ) at 6.4°, 16.3°, and 25.0°, and one or two of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 16.3°, and 25.0°, and one of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 16.3°, and 25.0°, and two of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°. In some embodiments, Formula I trans-ferulic co-crystal, Form I has an XRPD pattern comprising any three of the 2θ-reflections (+/−0.2 degrees 2θ) at 6.4°, 8.7°, 16.3°, 24.2°, 25.0°, and 28.8°.

In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one, two or three of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one or two of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one of the 2θ-reflections (+/−0.2 degrees 2θ) at 21.3°, 22.7°, and 26.9°. In some embodiments Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and two of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 21.3°, 22.7°, 24.2°, 25.0°, 26.9° and 28.8°. In some embodiments, Formula I trans-ferulic co-crystal, Form I has an XRPD pattern comprising any three of the 2θ-reflections (+/−0.2 degrees 2θ) at 6.4°, 8.7°, 16.3°, 21.3°, 22.7°, 24.2°, 25.0°, 26.9° and 28.8°.

In some embodiments, Formula I trans-ferulic co-crystal, Form I has an XRPD pattern comprising at least three of the 2θ-reflections (+/−0.2 degrees 2θ) at 6.4°, 8.7°, 10.7°, 14.2°, 15.1°, 16.3°, 18.9°, 21.3°, 22.7°, 24.2°, 25.0°, 26.9° and 28.8°. In some embodiments, Formula I trans-ferulic co-crystal, Form I has an XRPD pattern comprising at least four of the 2θ-reflections (+/−0.2 degrees 2θ) at 6.4°, 8.7°, 10.7°, 14.2°, 15.1°, 16.3°, 18.9°, 21.3°, 22.7°, 24.2°, 25.0°, 26.9° and 28.8°. In some embodiments Formula I trans-ferulic co-crystal, Form I has an XRPD pattern comprising at least five of the 2θ-reflections (+/−0.2 degrees 2θ) at 6.4°, 8.7°, 10.7°, 14.2°, 15.1°, 16.3°, 18.9°, 21.3°, 22.7°, 24.2°, 25.0°, 26.9° and 28.8°.

In some embodiments, Formula I trans-ferulic co-crystal, Form I has an XRPD pattern comprising peaks at:

| Pos. | Rel. Int. |
|---|---|
| [°2 Th.] | [%] |
| 6.4 | 100 |
| 7.6 | 9 |
| 8.7 | 23 |
| 10.7 | 14 |
| 13.7 | 9 |
| 14.2 | 11 |
| 15.1 | 14 |
| 16.3 | 38 |
| 18.9 | 10 |
| 19.5 | 6 |
| 21.3 | 20 |
| 22.7 | 15 |
| 24.2 | 31 |
| 25.0 | 74 |
| 26.9 | 19 |
| 28.0 | 7 |
| 28.8 | 28 |

In some embodiments, Formula I trans-ferulic co-crystal, Form I is characterized by a DSC thermogram substantially as shown in FIG. 52.

In some embodiments, Formula I trans-ferulic co-crystal, Form I is characterized by a DSC thermogram having one or both of (i) an endothermic transition at about 139° C. and (ii) an endothermic transition at about 180° C. In some embodiments, Formula I trans-ferulic co-crystal, Form I is characterized by a DSC thermogram having (i) an endothermic transition at about 139° C. and (ii) an endothermic transition at about 180° C.

In some embodiments, Formula I trans-ferulic co-crystal, Form I is characterized by a TGA curve substantially as shown in FIG. 53. In some embodiments, Formula I trans-ferulic co-crystal, Form I loses mass starting at about 115° C.

In some embodiments Formula I trans-ferulic co-crystal, Form I is characterized by a DVS curve substantially as shown in shown in FIG. 54. In some embodiments, Formula I trans-ferulic co-crystal, Form I absorbs less than about 0.35% of water up to 90% RH at 25° C.

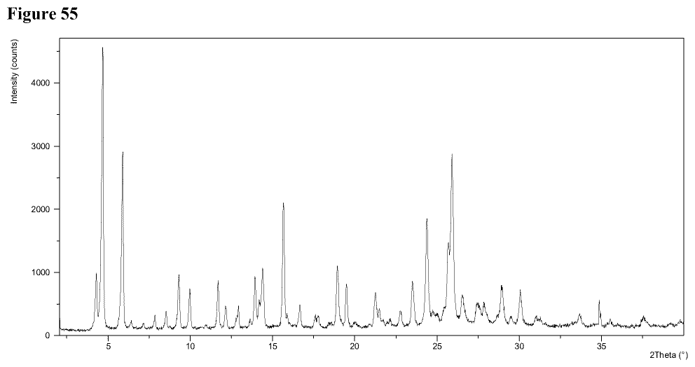

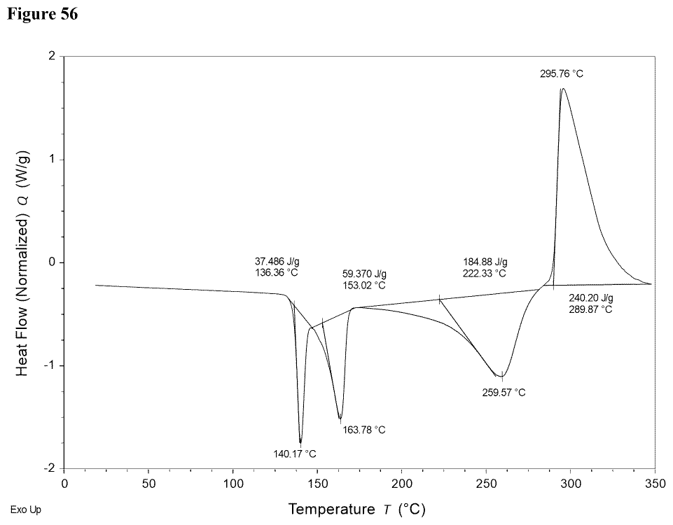

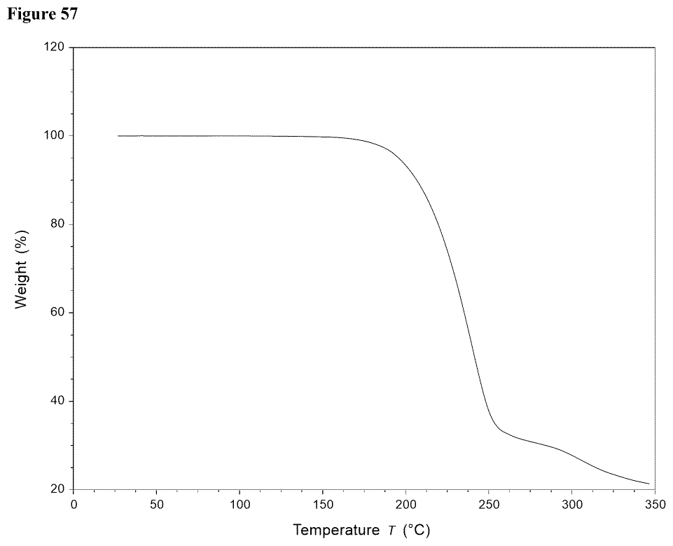

In some embodiments, the crystalline compound of Formula I, trans-ferulic co-crystal is a Form II (“compound of Formula I, trans-ferulic co-crystal, Form II” or “Formula I trans-ferulic co-crystal, Form II”). In some embodiments, compound of Formula I, trans-ferulic co-crystal, Form II has an XRPD profile substantially as shown in FIG. 55. The Formula I trans-ferulic co-crystal, Form II may exhibit a DSC thermogram substantially as shown in FIG. 56. The Formula I trans-ferulic co-crystal, Form II may exhibit a TGA graph substantially as shown in FIG. 57.

In some embodiments, the Formula I trans-ferulic co-crystal, Form II has an XRPD pattern displaying at least two, at least three, at least four, at least five, or at least six of the degree 2θ-reflections with the greatest intensity as the XRPD pattern substantially as shown in FIG. 55.

In some embodiments, the Formula I trans-ferulic co-crystal, Form II has an XRPD pattern comprising 2θ-reflections (+/−0.2 degrees 2θ) at 4.7°, 5.9°, and 25.9°. In some embodiments, the Formula I trans-ferulic co-crystal, Form II has an XRPD pattern comprising 2θ-reflections (+/−0.2 degrees 2θ) at 4.7°, 5.9°, and 25.9°, and one, two or three of the 2θ-reflections (+/−0.2 degrees 2θ) at 15.7°, 18.9°, and 24.4°. In some embodiments, the Formula I trans-ferulic co-crystal, Form II has an XRPD pattern comprising 2θ-reflections (+/−0.2 degrees 2θ) at 4.7°, 5.9°, and 25.9°, and one or two of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, and 25.9°, and one of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, and 25.9°, and two of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°. In some embodiments, Formula I trans-ferulic co-crystal, Form II has an XRPD pattern comprising any three of the 2θ-reflections (+/−0.2 degrees 2θ) at 4.7°, 5.9°, 15.7°, 18.9°, 24.4°, and 25.9°.

In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one, two or three of the 2θ-reflections (+/−0.2 degrees 2θ) at 4.3°, 9.3°, and 14.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one or two of the 2θ-reflections (+/−0.2 degrees 2θ) at 4.3°, 9.3°, and 14.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one of the 2θ-reflections (+/−0.2 degrees 2θ) at 4.3°, 9.3°, and 14.4°. In some embodiments Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and two of the 220-reflections (+/−0.2 degrees 2θ) at 4.3°, 9.3°, and 14.4°. In some embodiments, Formula I trans-ferulic co-crystal, Form II has an XRPD pattern comprising 2-reflections (+/−0.2 degrees 2θ) at 4.3°, 4.7°, 5.9°, 9.3°, 14.4°, 15.7°, 18.9°, 24.4°, and 25.9°. In some embodiments, Formula I trans-ferulic co-crystal, Form II has an XRPD pattern comprising any three of the 2θ-reflections (+/−0.2 degrees 2θ) at 4.3°, 4.7°, 5.9°, 9.3°, 14.4°, 15.7°, 18.9°, 24.4°, and 25.9°.

In some embodiments, Formula I trans-ferulic co-crystal, Form II has an XRPD pattern comprising at least three of the 2θ-reflections (+/−0.2 degrees 2θ) at 4.3°, 4.7°, 5.9°, 9.3°, 14.4°, 15.7°, 18.9°, 24.4°, and 25.9°. In some embodiments, Formula I trans-ferulic co-crystal, Form II has an XRPD pattern comprising at least four of the 2θ-reflections (+/−0.2 degrees 2θ) at 4.3°, 4.7°, 5.9°, 9.3°, 14.4°, 15.7°, 18.9°, 24.4°, and 25.9°. In some embodiments Formula I trans-ferulic co-crystal, Form II has an XRPD pattern comprising at least five of the 2θ-reflections (+/−0.2 degrees 2θ) at 4.3°, 4.7°, 5.9°, 9.3°, 14.4°, 15.7°, 18.9°, 24.4°, and 25.9°.

In some embodiments, Formula I trans-ferulic co-crystal, Form II has an XRPD pattern comprising peaks at:

| Pos. | Rel. Int. |
|---|---|
| [°2 Th.] | [%] |
| 4.3 | 20 |
| 4.7 | 100 |
| 5.9 | 63 |
| 9.3 | 19 |
| 11.7 | 17 |
| 14.4 | 21 |
| 15.7 | 45 |
| 16.7 | 8 |
| 18.9 | 22 |
| 19.5 | 15 |
| 21.3 | 6 |
| 23.5 | 16 |
| 24.4 | 37 |
| 25.9 | 60 |
| 27.4 | 6 |
| 28.9 | 13 |
| 30.0 | 11 |

In some embodiments, Formula I trans-ferulic co-crystal, Form II is characterized by a DSC thermogram substantially as shown in FIG. 56.

In some embodiments, Formula I trans-ferulic co-crystal, Form II is characterized by a DSC thermogram having at least one of (i) an endothermic transition at about 136° C., (ii) an endothermic transition at about 153° C., (iii) an endothermic transition at about 222° C., and (iv) an exothermic transition at about 290° C. In some embodiments, Formula I trans-ferulic co-crystal, Form II is characterized by a DSC thermogram having (i) an endothermic transition at about 136° C., (ii) an endothermic transition at about 153° C., (iii) an endothermic transition at about 222° C., and (iv) an exothermic transition at about 290° C.

In some embodiments, Formula I trans-ferulic co-crystal, Form II is characterized by a TGA curve substantially as shown in FIG. 57. In some embodiments, Formula I trans-ferulic co-crystal, Form II is characterized in being unsolvated.

### Tromethamine Salt of the Compound of Formula I

In some embodiments, pharmaceutical composition comprises a tromethamine salt of Formula I. In some embodiments, the tromethamine salt of the compound of Formula I is a tromethamine co-crystal (“compound of Formula I tromethamine co-crystal” or “Formula I tromethamine co-crystal”).

In some embodiments, the compound of Formula I tromethamine co-crystal is crystalline.

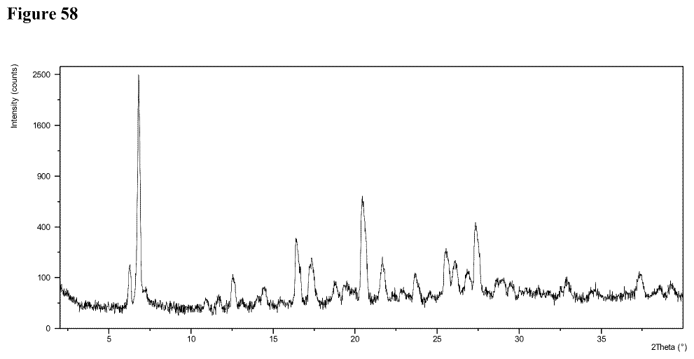

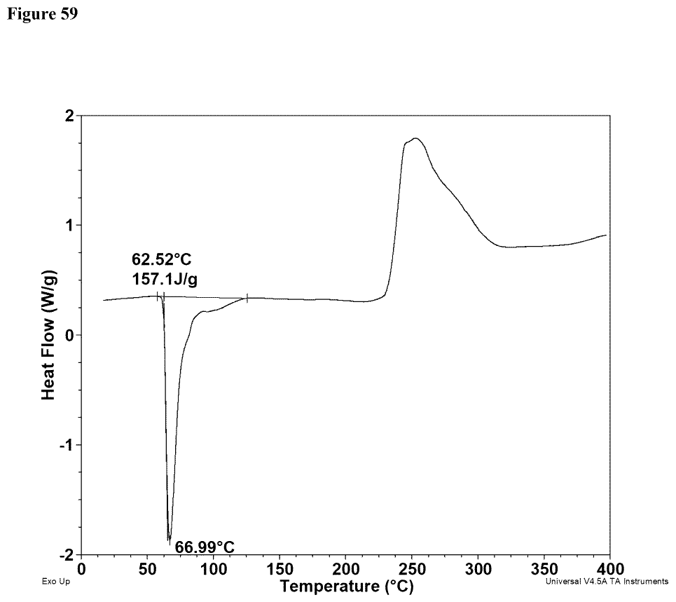

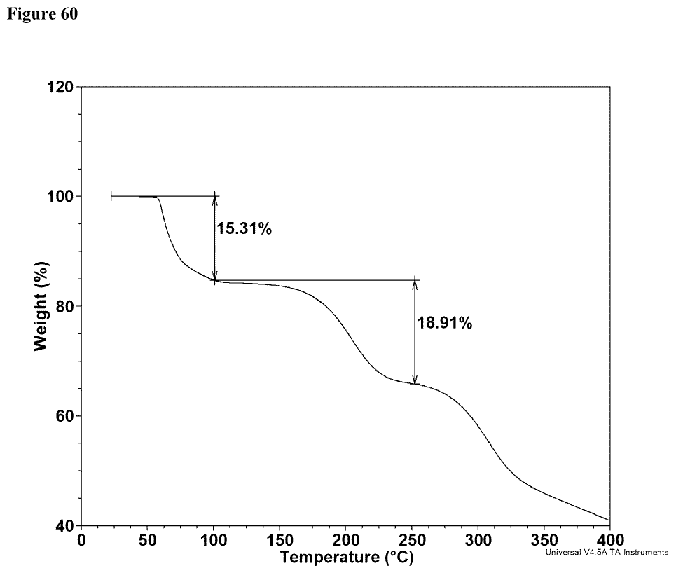

In some embodiments, the crystalline compound of Formula I, tromethamine co-crystal has an XRPD profile substantially as shown in FIG. 58. The crystalline compound of Formula I, tromethamine co-crystal may exhibit a DSC thermogram substantially as shown in FIG. 59. The crystalline compound of Formula I, tromethamine co-crystal may exhibit a TGA graph substantially as shown in FIG. 60.

In some embodiments, the crystalline compound of Formula I, tromethamine co-crystal has an XRPD pattern comprising 2θ-reflections (+/−0.2 degrees 2θ) at 6.8°, 20.4°, and 27.3°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one, two or three of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one or two of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and two of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°. In some embodiments, the crystalline compound of Formula I, tromethamine co-crystal has an XRPD pattern comprising any three of the 2θ-reflections (+/−0.2 degrees 2θ) at 6.8°, 16.4°, 17.3°, 20.4°, 25.5°, and 27.3°.

In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one, two or three of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one or two of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and two of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 12.6°, 16.4°, 17.3°, 20.4°, 21.6°, 23.7°, 25.5°, and 27.3°. In some embodiments, the crystalline compound of Formula I, tromethamine co-crystal has an XRPD pattern comprising any three of the 2θ-reflections (+/−0.2 degrees 2θ) at 6.8°, 12.6°, 16.4°, 17.3°, 20.4°, 21.6°, 23.7°, 25.5°, and 27.3°.

In some embodiments, the crystalline compound of Formula I, tromethamine co-crystal has an XRPD pattern comprising at least three of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°. In some embodiments, the crystalline compound of Formula I, tromethamine co-crystal has an XRPD pattern comprising at least four of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°. In some embodiments, the crystalline compound of Formula I, tromethamine co-crystal has an XRPD pattern comprising at least five of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°.

In some embodiments, the crystalline compound of Formula I, tromethamine co-crystal has an XRPD pattern comprising peaks at:

| Pos. | Rel. Int. |
|---|---|
| [°2 Th.] | [%] |
| 6.2 | 5 |
| 6.8 | 100 |
| 12.6 | 3 |
| 14.3 | 1 |
| 16.4 | 12 |
| 17.3 | 6 |
| 20.4 | 24 |
| 21.6 | 6 |
| 23.7 | 3 |
| 25.5 | 7 |
| 26.8 | 3 |
| 27.3 | 15 |
| 32.9 | 2 |
| 37.3 | 3 |

In some embodiments, the crystalline compound of Formula I, tromethamine co-crystal is characterized by a DSC thermogram substantially as shown in FIG. 59.

In some embodiments, the crystalline compound of Formula I, tromethamine co-crystal is characterized by a DSC thermogram having endothermic transition with an onset at about 63° C.

In some embodiments, the crystalline compound of Formula I, tromethamine co-crystal is characterized by a TGA curve substantially as shown in FIG. 60. In some embodiments, the crystalline compound of Formula I, tromethamine co-crystal loses mass in multiple stages starting at about 50° C. and another stage starting at about 140° C.

The compound of Formula I can be present in the pharmaceutical compositions described herein in solvated and/or unsolvated form, and references to “a compound of Formula I” or “a compound of Formula I, or a pharmaceutically acceptable salt thereof” comprise the solvated and unsolvated forms and mixtures thereof. As used herein, and in absence of a specific reference to a particular pharmaceutically acceptable salt and/or solvate of the compound of Formula I, any dosages, whether expressed in, e.g., milligrams or as % by weight, should be taken as referring to the amount of the compound of Formula I, i.e., the amount of:

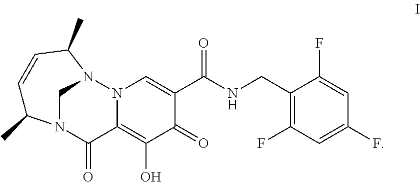

Therefore, for example, a reference to “500 mg of the compound of Formula I, or a pharmaceutically acceptable salt thereof” means an amount of the Compound of Formula I, or a pharmaceutically acceptable salt thereof, which provides the same amount as 500 mg of the free acid of the compound of Formula I.

The compound of Formula I, or pharmaceutically acceptable salt thereof, can have any suitable purity. For example, the compound of Formula I, or pharmaceutically acceptable salt thereof, can have a purity of at least about 90%, or at least about 91%, about 92%, about 93%, about 94%, about 95%, about 96%, about 97%, about 98%, about 99%, or at least about 99.1%, about 99.2%, about 99.3%, about 99.4%, about 99.5%, about 99.6%, about 99.7%, about 99.8% or at least about 99.9%. In some embodiments, the compound of Formula I, or pharmaceutically acceptable salt thereof, has a purity of at least about 99.1%. In some embodiments, the compound of Formula I, or pharmaceutically acceptable salt thereof, has a purity of at least about 99.3%. In some embodiments, the compound of Formula I, or pharmaceutically acceptable salt thereof, has a purity of at least about 99.5%. In some embodiments, the compound of Formula I, or pharmaceutically acceptable salt thereof, has a purity of at least about 99.7%. In some embodiments, the compound of Formula I, or pharmaceutically acceptable salt thereof, has a purity of at least about 99.5%. In some embodiments, the compound of Formula I, or pharmaceutically acceptable salt thereof, has a purity of at least about 99.9%.

In some embodiments, the compound of Formula I, or pharmaceutically acceptable salt thereof, is micronized. Micronization is the process of reducing the particle size of a bulk solid material (e.g., a pharmaceutical drug substance) to micron or submicron level. Micronization of pharmaceutical bulk solids can be achieved via methods including, for example, milling or grinding.

In some embodiments, the particle size of the compound of Formula I, or pharmaceutically acceptable salt thereof, is controlled by milling, e.g., jet milling (fluid energy milling), bead milling, dry milling spiral milling, or high shear wet milling (HSWM). In an embodiment, the particle size of the compound of Formula I, or pharmaceutically acceptable salt thereof, is controlled by high shear wet milling. High shear wet milling can be either integrated into the crystallization process or performed post crystallization. In some embodiments, the HSWM is performed at a single speed of about 3,000 rpm to about 20,000 rpm; at about 3,000 rpm, about 4,000 rpm, about 5,000 rpm, about 6,000 rpm, about 7,000 rpm, about 8,000 rpm, about 9,000 rpm, about 10,000 rpm, about 11,000 rpm, about 12,000 rpm, about 13,000 rpm, about 14,000 rpm, about 15,000 rpm, or about 16,000 rpm. In an embodiment, the HSWM is performed at a single speed of 4,200 rpm. In an embodiment, the HSWM is performed at a single speed of 5,500 rpm. In an embodiment, the HSWM is performed at a single speed of 8,000 rpm. In an embodiment, the HSWM is performed at a single speed of 12,000 rpm. In an embodiment, the HSWM is performed at a single speed of 16,000 rpm. In some embodiments, the wet-milled granulated particles are passed through a comil upon drying. In some embodiments, the size of the comil screen is about 0.032 inches to about 0.250 inches. In an embodiment, the size of the comil screen is about 0.032 inches. In an embodiment, the size of the comil screen is about 0.250 inches. In some embodiments, the wet-milled granulated particles are dried in a tumble dryer. In some embodiments, the wet-milled granulated particles are dried in an agitated filter/dryer. In some embodiments, the wet-milled granulated particles are dried in a vacuum oven dryer.

In an embodiment, the particle size of the compound of Formula I, or pharmaceutically acceptable salt thereof, is controlled by jet milling. In an embodiment, the particle size of the compound of Formula I, or pharmaceutically acceptable salt thereof, is controlled by loop style jet milling. In an embodiment, the particle size of the compound of Formula I, or pharmaceutically acceptable salt thereof, is controlled by a Model 0202 Jet-O-Mizer (JOM) loop style jet mill system. In an embodiment, the particle size of the compound of Formula I, or pharmaceutically acceptable salt thereof, is controlled by spiral style jet milling.

The particle size distribution of a solid material can be measured using a variety of analytical characterization methods known to those skilled in the art, including, for example, sieving, laser light diffraction, quasi-elastic light scattering, centrifugal sedimentation-optical, electrical resistance zone sensing, microelectrophoresis, light microscopy, and scanning electron microscopy, etc. In some embodiments, the particle size distribution of the compound of Formula I, or pharmaceutically acceptable salt thereof, is measured using light microscopy. In some embodiments, the particle size distribution of the compound of Formula I, or pharmaceutically acceptable salt thereof, is measured using laser light diffraction. The particle size distribution of a solid material can be represented by the d₉₀ values as defined herein.

In some embodiments, the compound of Formula I, or pharmaceutically acceptable salt thereof, has a d₉₀ value of less than about 800 μm, less than about 300 μm, or less than about 100 μm. In some embodiments, the compound of Formula I, or pharmaceutically acceptable salt thereof, has a particle size distribution with a d₉₀ value less than about 200 μm. For example, the compound of Formula I, or pharmaceutically acceptable salt thereof, has a particle size distribution with a do value less than about 150 μm, about 125 μm, about 100 μm, about 95 μm, about 90 μm, about 85 μm, about 80 μm, about 70 μm, about 65 μm, about 60 μm, about 55 μm, about 50 μm, about 45 μm, about 40 μm, about 35 μm, about 30 μm, about 25 μm, about 20 μm, about 15 μm, about 10 μm, about 9 μm, about 8 μm, about 7 μm, about 6 μm, about 5 μm, about 4 μm, about 3 μm, about 2 μm, or about 1 μm. In some embodiments, the compound of Formula I, or pharmaceutically acceptable salt thereof, has a particle size distribution with a do value from about 1 μm to about 150 μm, for example, about 1 μm to about 125 μm, about 1 μm to about 100 μm, about 1 μm to about 95 μm, about 1 μm to about 90 μm, about 1 μm to about 85 μm, about 1 μm to about 80 μm, about 1 μm to about 75 μm, about 1 μm to about 70 μm, about 1 μm to about 15 μm, about 5 μm to about 60 μm, about 1 μm to about 55 μm, about 1 μm to about 50 μm, about 1 μm to about 45 μm, about 1 μm to about 40 μm, about 1 μm to about 35 μm, about 1 μm to about 30 μm, about 1 μm to about 25 μm, about 1 μm to about 20 μm, about 1 μm to about 15 μm, about 1 μm to about 10 μm, 1 μm to about 9 μm, 1 μm to about 8 μm, 1 μm to about 7 μm, 1 μm to about 6 μm, 1 μm to about 5 μm, 1 μm to about 4 μm, 1 μm to about 3 μm, or 1 μm to 2 μm. In some embodiments, the compound of Formula I, or pharmaceutically acceptable salt thereof, has a particle size distribution with a d₉₀ value of about 55 μm. In some embodiments, the compound of Formula I, or pharmaceutically acceptable salt thereof, has a particle size distribution with a d₉₀ value of about 53 μm. In some embodiments, the compound of Formula I, or pharmaceutically acceptable salt thereof, has a particle size distribution with a d₉₀ value of about 52 μm. In some embodiments, the compound of Formula I, or pharmaceutically acceptable salt thereof, has a particle size distribution with a d₉₀ value of about 25 μm. In some embodiments, the compound of Formula I, or pharmaceutically acceptable salt thereof, has a particle size distribution with a d₉₀ value of about 24 μm. In some embodiments, the compound of Formula I, or pharmaceutically acceptable salt thereof, has a particle size distribution with a d₉₀ value of about 14 μm. In some embodiments, the compound of Formula I, or pharmaceutically acceptable salt thereof, has a particle size distribution with a d₉₀ value of about 11 μm. In some embodiments, the compound of Formula I, or pharmaceutically acceptable salt thereof, has a particle size distribution with a d₉₀ value of ≤about 9 μm.

In some embodiments, the compound of Formula I, or pharmaceutically acceptable salt thereof, has a particle size distribution with a d₉₀ value of ≤about 30 μm. In some embodiments, the compound of Formula I, or pharmaceutically acceptable salt thereof, has a particle size distribution with a d₉₀ value of ≤about 20 μm. In some embodiments, the compound of Formula I, or pharmaceutically acceptable salt thereof, has a particle size distribution with a d₉₀ value of ≤about 15 μm. In some embodiments, the compound of Formula I, or pharmaceutically acceptable salt thereof, has a particle size distribution with a d₉₀ value of ≤about 10 μm.

In some embodiments, the compound of Formula I, or pharmaceutically acceptable salt thereof, has a particle size distribution with a d₉₀ value from about 1 μm to about 150 μm. In some embodiments, the compound of Formula I, or pharmaceutically acceptable salt thereof, has a particle size distribution with a d₉₀ value from about 5 μm to about 125 μm. In some embodiments, the compound of Formula I, or pharmaceutically acceptable salt thereof, has a particle size distribution with a do value from about 14 μm to about 113 μm. In some embodiments, the compound of Formula I, or pharmaceutically acceptable salt thereof, has a particle size distribution with a d₉₀ value from about 5 μm to about 60 μm. In some embodiments, the compound of Formula I, or pharmaceutically acceptable salt thereof, has a particle size distribution with a d₉₀ value from about 5 μm to about 15 μm.

In some embodiments, the compound of Formula I, or pharmaceutically acceptable salt thereof, is processed by a solvent evaporation method (e.g., spray drying, lyophilization, super-critical fluid, co-precipitation, electrospinning). In some embodiments, the compound of Formula I, or a pharmaceutically acceptable salt thereof, is spray dried.

### III. Pharmaceutical Formulations

The pharmaceutical formulations described herein comprise the compound of Formula I, or a pharmaceutically acceptable salt thereof, and one or more excipients. These excipients may be, for example, inert diluents, fillers, granulating agents, disintegrants, binders, lubricants, surfactants, pH modifiers, and/or glidants. In some embodiments, the one or more excipients comprise a filler, a disintegrant, a lubricant, a surfactant, a pH modifier, a binder, or combinations thereof. In some embodiments, the pharmaceutical formulations disclosed herein comprise the compound of Formula I, or a pharmaceutically acceptable salt thereof, and one or more excipients selected from a filler, a disintegrant, a lubricant, a surfactant, a pH modifier, and a binder. In some embodiments, the pharmaceutical formulations disclosed herein comprise the compound of Formula I, or a pharmaceutically acceptable salt thereof, a filler, a disintegrant, a binder, and a lubricant.

The pharmaceutical compositions disclosed herein may be presented as discrete units (a unit dosage form) including but not limited to capsules, cachets or tablets each containing a predetermined amount of the active ingredient. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation.

In some embodiments, the pharmaceutical formulations disclosed herein are a solid dosage form. The solid dosage forms disclosed herein comprise one or more excipients. Excipients should be compatible with the other ingredients of the formulation and physiologically innocuous to the recipient thereof. Examples of suitable excipients may be found e.g., in Handbook of Pharmaceutical Excipients (eds. Rowe, Sheskey & Quinn), 6th edition 2009.

The pharmaceutical formulations disclosed herein are for oral administration to a subject (for e.g., a human). As such, provided herein are solid dosage forms of the compound of Formula I for oral administration (“solid oral dosage forms”).

In some embodiments, disclosed herein are solid oral dosage forms comprising the compound of Formula I, or pharmaceutically acceptable salt thereof, and one or more excipients. The solid oral dosage forms (e.g., tablets) may be prepared from hot melt extrusion or spray-drying dispersion (SDD) technologies. In some embodiments, the oral dosage forms are prepared by spray-drying dispersion with one or more polymers (e.g., copovidone, povidone, hypromellose, hypromellose phthalate, hypromellose acetate succinate, cellulose acetate phthalate, methacrylic acid-ethyl acrylate copolymer, or combinations thereof).

In some embodiments, the compound of Formula I, or a pharmaceutically acceptable salt thereof, is spray dried in a dispersion with one or more polymers. In some embodiments, the one or more polymers comprise neutral or charged polymers. In some embodiments, the one or more polymers are selected from copovidone, povidone, hypromellose, hypromellose phthalate, hypromellose acetate succinate, cellulose acetate phthalate, (meth)acrylate polymers, or combinations thereof. In some embodiments, the (meth)acrylate polymers comprise methacrylic acid-methyl methacrylate copolymer, methacrylic acid-ethyl acrylate copolymer, or a combination thereof. In some embodiments, the one or more polymers comprise copovidone.

In some embodiments, the compound of Formula I, or a pharmaceutically acceptable salt thereof, is spray dried in a dispersion with one or more polymers, wherein the dispersion comprises about 40% to about 60% of the compound of Formula I, or a pharmaceutically acceptable salt thereof, by weight and about 40% to about 60% of the one or more polymers by weight. In some embodiments, the dispersion comprises about 50% of the compound of Formula I, or a pharmaceutically acceptable salt thereof, by weight and about 50% of the one or more polymers by weight.

In some embodiments, the solid oral dosage form is a tablet or a capsule. In some embodiments, the solid oral dosage form is a tablet. In some embodiments, disclosed herein are tablets containing the active ingredient in admixture with one or more excipients which are suitable for manufacture of tablets. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the solid oral dosage form is a capsule. In some embodiments, disclosed herein are hard capsules filled with powder, beads, or granules containing the active ingredient in admixture with one or more excipients which are suitable for manufacture of hard or soft capsules.

In some embodiments, the pharmaceutical compositions (e.g., solid oral dosage forms) disclosed herein comprise one or more excipients selected from a filler, a disintegrant, a binder and a lubricant. In some embodiments, the one or more excipients comprise a filler, a disintegrant, and a lubricant. In some embodiments, the one or more excipients comprise a filler, a disintegrant, a lubricant, and a binder.

The pharmaceutical compositions disclosed herein may have desirable properties for medical or pharmaceutical use. Such properties can include manufacturability (e.g., compressibility, ease of handling, ability to constantly prepare doses of the same strength, etc.), physical stability (e.g., thermal stability, shelf life, etc.), chemical stability, drug loading, dissolution rate (e.g., bioavailability) and process control. Thus, the present pharmaceutical compositions (e.g. tablets) may provide advantages such as improving the manufacturing process of the compound, improving the stability or storability of a drug product form of the compound, or improving drug loading of the compound.

### The Compound of Formula I

The pharmaceutical compositions (e.g., solid oral dosage forms) described herein utilize the compound of Formula I, wherein the compound of Formula I is in the form of a free acid and/or a pharmaceutically acceptable salt thereof.

The compound of Formula I, or pharmaceutically acceptable salt thereof, can be in any suitable form (e.g., amorphous or crystalline). In some embodiments of the oral dosage forms disclosed herein, the dosage form comprises the compound of Formula I. In some embodiments of the oral dosage forms disclosed herein, the compound of Formula I is crystalline. In some embodiments of the oral dosage forms disclosed herein, the compound of Formula I is crystalline form I. In some embodiments of the oral dosage forms disclosed herein, the compound of Formula I is amorphous.

In some embodiments of the oral dosage forms disclosed herein, the dosage form comprises a pharmaceutically acceptable salt of the compound of Formula I. In some embodiments of the oral dosage forms disclosed herein, the dosage form comprises a sodium salt of the compound of Formula I. In some embodiments of the oral dosage forms disclosed herein, the dosage form comprises an n-butylamine salt of the compound of Formula I. In some embodiments of the oral dosage forms disclosed herein, the dosage form comprises an ammonia salt of the compound of Formula I. In some embodiments of the oral dosage forms disclosed herein, the dosage form comprises a tromethamine salt of the compound of Formula I. In some embodiments of the oral dosage forms disclosed herein, the dosage form comprises a potassium salt of the compound of Formula I. In some embodiments of the oral dosage forms disclosed herein, the dosage form comprises a diethylamine salt of the compound of Formula I. In some embodiments of the oral dosage forms disclosed herein, the dosage form comprises a calcium salt of the compound of Formula I. In some embodiments of the oral dosage forms disclosed herein, the dosage form comprises a magnesium salt of the compound of Formula I. In some embodiments of the oral dosage forms disclosed herein, the dosage form comprises a diethanolamine salt of the compound of Formula I. In some embodiments of the oral dosage forms disclosed herein, the dosage form comprises an ethylenediamine salt of the compound of Formula I. In some embodiments of the oral dosage forms disclosed herein, the dosage form comprises a morpholine salt of the compound of Formula I. In some embodiments of the oral dosage forms disclosed herein, the dosage form comprises an L-arginine salt of the compound of Formula I. In some embodiments of the oral dosage forms disclosed herein, the dosage form comprises a trans-Ferulic acid salt of the compound of Formula I. In some embodiments of the oral dosage forms disclosed herein, the salt of the compound of Formula I is crystalline. In some embodiments of the oral dosage forms disclosed herein, the salt of the compound of Formula I is amorphous.

The pharmaceutical compositions (e.g., solid oral dosage forms) disclosed herein may comprise any suitable amount of the compound of Formula I, or pharmaceutically acceptable salt thereof, for example about 0.1 mg to about 2000 mg of the compound of Formula I, or the pharmaceutically acceptable salt thereof, wherein the amount is based on the free acid form. In some embodiments, the compound of Formula I, or pharmaceutically acceptable salt thereof, is present in an amount of about 1 mg to about 1500 mg, about 5 mg to about 1400 mg, about 5 mg to about 750 mg, about 5 mg to about 300 mg, about 5 mg to about 100 mg, about 10 mg to about 500 mg, about 10 mg to about 400 mg, about 10 mg to about 200 mg, about 10 mg to about 100 mg, about 10 mg to about 50 mg, about 10 mg to about 30 mg, about 50 mg to about 1000 mg, about 50 mg to about 800 mg, about 50 mg to about 600 mg, about 50 mg to about 400 mg, about 50 mg to about 300 mg, about 50 mg to about 100 mg, about 100 mg to about 1000 mg, about 100 mg to about 800 mg, about 100 mg to about 600 mg, about 100 mg to about 400 mg, about 100 mg to about 200 mg, about 200 mg to about 1000 mg, about 200 mg to about 800 mg, about 200 mg to about 700 mg, about 200 mg to about 600 mg, about 200 mg to about 400 mg, about 200 mg to about 300 mg, about 300 mg to about 1000 mg, about 300 mg to about 800 mg, about 300 mg to about 600 mg, about 300 mg to about 500 mg, about 300 mg to about 400 mg, about 400 mg to about 1000 mg, about 400 mg to about 800 mg, about 400 mg to about 700 mg, about 400 mg to about 600 mg, about 400 mg to about 500 mg, about 500 mg to about 1000 mg, about 500 mg to about 800 mg, about 500 mg to about 600 mg, about 600 mg to about 1000 mg, about 600 mg to about 900 mg, about 600 mg to about 800 mg, about 600 mg to about 700 mg, about 700 mg to about 1000 mg, about 700 mg to about 900 mg, about 700 mg to about 800 mg, about 800 mg to about 1000 mg, about 800 mg to about 900 mg, about 900 mg to about 1000 mg, about 200 mg to about 1500 mg, about 200 mg to about 1400 mg, about 400 mg to about 1300 mg, about 600 mg to about 1350 mg, about 800 mg to about 1200 mg, about 800 mg to about 1000 mg, about 1000 mg to about 1500 mg, about 1000 mg to about 1200 mg, or about 1200 mg to about 1400 mg, wherein the amount is based on the free acid form. In some embodiments, the compound of Formula I, or pharmaceutically acceptable salt thereof, is present in an amount of about 5 mg to about 1400 mg, wherein the amount is based on the free acid form.

In some embodiments, provided herein is a solid dosage form comprising a compound of Formula I, or a pharmaceutically acceptable salt thereof, and one or more excipients; wherein the compound of Formula I, or pharmaceutically acceptable salt thereof, is present in an amount of about 5 mg to about 1400 mg, wherein the amount is based on the free acid form. In some embodiments, the compound of Formula I, or pharmaceutically acceptable salt thereof, is present in an amount of about 5 mg to about 1350 mg, wherein the amount is based on the free acid form. In some embodiments, the compound of Formula I, or pharmaceutically acceptable salt thereof, is present in an amount of about 5 mg to about 750 mg, wherein the amount is based on the free acid form. In some embodiments, the compound of Formula I, or pharmaceutically acceptable salt thereof, is present in an amount of about 5 mg to about 300 mg, wherein the amount is based on the free acid form. In some embodiments, the compound of Formula I, or pharmaceutically acceptable salt thereof, is present in an amount of about 5 mg to about 100 mg, wherein the amount is based on the free acid form. In some embodiments, the compound of Formula I, or pharmaceutically acceptable salt thereof, is present in an amount of about 10 mg to about 250 mg, wherein the amount is based on the free acid form. In some embodiments, the compound of Formula I, or pharmaceutically acceptable salt thereof, is present in an amount of about 100 mg to about 200 mg, wherein the amount is based on the free acid form. In some embodiments, the compound of Formula I, or pharmaceutically acceptable salt thereof, is present in an amount of about 200 mg to about 300 mg, wherein the amount is based on the free acid form. In some embodiments, the compound of Formula I, or pharmaceutically acceptable salt thereof, is present in an amount of about 300 mg to about 500 mg, wherein the amount is based on the free acid form. In some embodiments, the compound of Formula I, or pharmaceutically acceptable salt thereof, is present in an amount of about 300 mg to about 400 mg, wherein the amount is based on the free acid form. In some embodiments, the compound of Formula I, or pharmaceutically acceptable salt thereof, is present in an amount of about 400 mg to about 700 mg, wherein the amount is based on the free acid form. In some embodiments, the compound of Formula I, or pharmaceutically acceptable salt thereof, is present in an amount of about 400 mg to about 500 mg, wherein the amount is based on the free acid form. In some embodiments, the compound of Formula I, or pharmaceutically acceptable salt thereof, is present in an amount of about 600 mg to about 700 mg, wherein the amount is based on the free acid form.

In some embodiments, the compound of Formula I, or pharmaceutically acceptable salt thereof, is present in an amount of about 5 mg to about 500 mg, wherein the amount is based on the free acid form. In some embodiments, the compound of Formula I, or pharmaceutically acceptable salt thereof, is present in an amount of about 10 mg to about 450 mg, wherein the amount is based on the free acid form.

In some embodiments, the oral dosage form comprises the compound of Formula I in an amount of about 5 mg to about 500 mg. In some embodiments, the oral dosage form comprises the compound of Formula I in an amount of about 5 mg to about 500 mg, wherein the compound of Formula I is crystalline Form I.

In some embodiments, the oral dosage form comprises the compound of Formula I in an amount of about 10 mg to about 450 mg. In some embodiments, the oral dosage form comprises the compound of Formula I in an amount of about 10 mg to about 450 mg, wherein the compound of Formula I is crystalline Form I.

In some embodiments, the oral dosage form comprises the compound of Formula I in an amount of about 10 mg to about 250 mg. In some embodiments, the oral dosage form comprises the compound of Formula I in an amount of about 10 mg to about 250 mg, wherein the compound of Formula I is crystalline Form I.

In some embodiments, the compound of Formula I, or pharmaceutically acceptable salt thereof, is present in an amount of about 10 mg, wherein the amount is based on the free acid form. In some embodiments, the compound of Formula I, or pharmaceutically acceptable salt thereof, is present in an amount of about 40 mg, wherein the amount is based on the free acid form. In some embodiments, the compound of Formula I, or pharmaceutically acceptable salt thereof, is present in an amount of about 50 mg, wherein the amount is based on the free acid form. In some embodiments, the compound of Formula I, or pharmaceutically acceptable salt thereof, is present in an amount of about 62.5 mg, wherein the amount is based on the free acid form. In some embodiments, the compound of Formula I, or pharmaceutically acceptable salt thereof, is present in an amount of about 250 mg, wherein the amount is based on the free acid form. In some embodiments, the compound of Formula I, or pharmaceutically acceptable salt thereof, is present in an amount of about 325 mg, wherein the amount is based on the free acid form. In some embodiments, the compound of Formula I, or pharmaceutically acceptable salt thereof, is present in an amount of about 450 mg, wherein the amount is based on the free acid form. In some embodiments, the compound of Formula I, or pharmaceutically acceptable salt thereof, is present in an amount of about 650 mg, wherein the amount is based on the free acid form.

In some embodiments, the oral dosage form comprises the compound of Formula I in an amount of about 10 mg. In some embodiments, the oral dosage form comprises the compound of Formula I in an amount of about 10 mg, wherein the compound of Formula I is crystalline Form I.

In some embodiments, the oral dosage form comprises the compound of Formula I in an amount of about 50 mg. In some embodiments, the oral dosage form comprises the compound of Formula I in an amount of about 50 mg, wherein the compound of Formula I is crystalline Form I.

In some embodiments, the oral dosage form comprises the compound of Formula I in an amount of about 250 mg. In some embodiments, the oral dosage form comprises the compound of Formula I in an amount of about 250 mg, wherein the compound of Formula I is crystalline Form I.

In some embodiments, the oral dosage form comprises the compound of Formula I in an amount of about 325 mg. In some embodiments, the oral dosage form comprises the compound of Formula I in an amount of about 325 mg, wherein the compound of Formula I is crystalline Form I.

In some embodiments, the oral dosage form comprises the compound of Formula I in an amount of about 450 mg. In some embodiments, the oral dosage form comprises the compound of Formula I in an amount of about 450 mg, wherein the compound of Formula I is crystalline Form I.

In some embodiments of the pharmaceutical compositions disclosed herein, the pharmaceutical composition comprises about 1 wt. % to about 50 wt. % of the compound of Formula I, or pharmaceutically acceptable salt thereof. For example, the pharmaceutical composition comprises about 5% to about 45%, about 1% to about 30%, about 30% to about 50%, about 1% to about 20%, about 5% to about 10%, about 10% to about 30%, about 20% to about 40%, about 5% to about 25%, about 15% to about 35%, or about 25% to about 35% of the compound of Formula I, or a pharmaceutically acceptable salt thereof. In some embodiments, the pharmaceutical composition comprises about 5 wt. % to about 35 wt. % of the compound of Formula I, or a pharmaceutically acceptable salt thereof. In some embodiments, the pharmaceutical composition comprises about 5 wt. % to about 35 wt. % of the compound of Formula I. In some embodiments, the pharmaceutical composition comprises about 5 wt. % to about 10 wt. % of the compound of Formula I. In some embodiments, the pharmaceutical composition comprises about 7.4 wt. % of the compound of Formula I. In some embodiments, the pharmaceutical composition comprises about 28 wt. % to about 32 wt. % of the compound of Formula I. In some embodiments, the pharmaceutical composition comprises about 30 wt. % of the compound of Formula I.

In some embodiments of the solid oral dosage forms disclosed herein, the compound of Formula I, or pharmaceutically acceptable salt thereof, is present in an amount of about 1% to about 50% of the total weight of the dosage form (i.e., wt. %). For example, the compound of Formula I, or pharmaceutically acceptable salt thereof, is present in an amount of about 5% to about 45%, about 1% to about 30%, about 30% to about 50%, about 1% to about 20%, about 5% to about 10%, about 10% to about 30%, about 20% to about 40%, about 5% to about 25%, about 15% to about 35%, or about 25% to about 35% of the total weight of the dosage form. In some embodiments, the compound of Formula I, or pharmaceutically acceptable salt thereof, is present in an amount of about 5% to about 45% of the total weight of the dosage form.

In some embodiments, the compound of Formula I, or pharmaceutically acceptable salt thereof, is present in an amount of about 20% to about 40% of the total weight of the dosage form. In some embodiments, the compound of Formula I, or pharmaceutically acceptable salt thereof, is present in an amount of about 20% of the total weight of the dosage form.

In some embodiments, the compound of Formula I, or pharmaceutically acceptable salt thereof, is present in an amount of about 25% to about 35% of the total weight of the dosage form. In some embodiments, the compound of Formula I, or pharmaceutically acceptable salt thereof, is present in an amount of about 30% of the total weight of the dosage form.

In some embodiments, the compound of Formula I, or pharmaceutically acceptable salt thereof, is present in an amount of about 5% to about 10% of the total weight of the dosage form. In some embodiments, the compound of Formula I, or pharmaceutically acceptable salt thereof, is present in an amount of about 7% of the total weight of the dosage form.

In some embodiments, the oral dosage form comprises about 5 wt. % to about 35 wt. % of the compound of Formula I, or a pharmaceutically acceptable salt thereof. In some embodiments, the oral dosage form comprises about 5 wt. % to about 35 wt. % of the compound of Formula I. In some embodiments, the oral dosage form comprises about 5 wt. % to about 35 wt. % of the compound of Formula I, wherein the compound of Formula I is crystalline Form I.

In some embodiments, the oral dosage form comprises about 5 wt. % to about 10 wt. % of the compound of Formula I, or a pharmaceutically acceptable salt thereof. In some embodiments, the oral dosage form comprises about 5 wt. % to about 10 wt. % of the compound of Formula I. In some embodiments, the oral dosage form comprises about 5 wt. % to about 10 wt. % of the compound of Formula I, wherein the compound of Formula I is crystalline Form I.

In some embodiments, the oral dosage form comprises about 7.4 wt. % of the compound of Formula I, or a pharmaceutically acceptable salt thereof. In some embodiments, the oral dosage form comprises about 7.4 wt. % of the compound of Formula I. In some embodiments, the oral dosage form comprises about 7.4 wt. % of the compound of Formula I, wherein the compound of Formula I is crystalline Form I.

In some embodiments, the oral dosage form comprises about 28 wt. % to about 32 wt. % of the compound of Formula I, or a pharmaceutically acceptable salt thereof. In some embodiments, the oral dosage form comprises about 28 wt. % to about 32 wt. % of the compound of Formula I. In some embodiments, the oral dosage form comprises about 28 wt. % to about 32 wt. % of the compound of Formula I, wherein the compound of Formula I is crystalline Form I.

In some embodiments, the oral dosage form comprises about 30 wt. % of the compound of Formula I, or a pharmaceutically acceptable salt thereof. In some embodiments, the oral dosage form comprises about 30 wt. % of the compound of Formula I. In some embodiments, the oral dosage form comprises about 30 wt. % of the compound of Formula I, wherein the compound of Formula I is crystalline Form I.

### Filler

In some embodiments of the pharmaceutical compositions (e.g., solid oral dosage forms) disclosed herein, the one or more excipients comprise a filler (i.e., a bulking agent or diluent). Examples of fillers that can be used in the pharmaceutical compositions (e.g., solid oral dosage forms) disclosed herein include, but are not limited to, starches, maltodextrins, polyols (such as lactose), celluloses, carbonates (such as calcium carbonate or sodium carbonate), phosphates (such as calcium phosphate or sodium phosphate) and povidone. In some embodiments, the filler comprises microcrystalline cellulose, lactose, mannitol, calcium phosphate, or a combination thereof. In some embodiments, the filler comprises lactose. In some embodiments, the lactose is lactose monohydrate. In some embodiments, the filler comprises microcrystalline cellulose. In some embodiments, the filler comprises lactose and microcrystalline cellulose.

In some embodiments of the pharmaceutical compositions disclosed herein, the pharmaceutical composition comprises from about 1 wt. % to about 95 wt. % of a filler. In some embodiments, the pharmaceutical composition comprises about 20 wt. % to about 90 wt. %, about 30 wt. % to about 90 wt. %, about 40 wt. % to about 90 wt. %, about 50 wt. % to about 90 wt. %, about 60 wt. % to about 90 wt. %, about 70 wt. % to about 90 wt. %, about 80 wt. % to about 90 wt. %, about 45 wt. % to about 95 wt. %, about 55 wt. % to about 95 wt. %, about 65 wt. % to about 95 wt. %, about 75 wt. % to about 95 wt. %, about 85 wt. % to about 95 wt. %, about 50 wt. % to about 80 wt. %, about 50 wt. % to about 70 wt. %, about 50 wt. % to about 60 wt. %, about 60 wt. % to about 80 wt. %, about 60 wt. % to about 70 wt. %, about 70 wt. % to about 80 wt. %, about 45 wt. % to about 85 wt. %, about 45 wt. % to about 75 wt. %, about 45 wt. % to about 65 wt. %, about 55 wt. % to about 85 wt. %, about 55 wt. % to about 75 wt. %, about 55 wt. % to about 65 wt. %, about 55 wt. % to about 60 wt. %, about 65 wt. % to about 85 wt. %, about 6 wt. 5% to about 75 wt. %, about 75 wt. % to about 85 wt. %, or about 80 wt. % to about 8 wt. 5% of the filler. In some embodiments the pharmaceutical composition comprises about 50 wt. % to about 90 wt. % of a filler.

In some embodiments of the solid oral dosage forms disclosed herein, the filler is present in an amount of about 1% to about 95% of the total weight of the dosage form (i.e., wt. %). In some embodiments, the filler is present in an amount of about 20% to about 90%, about 30% to about 90%, about 40% to about 90%, about 50% to about 90%, about 60% to about 90%, about 60% to about 82%, about 70% to about 90%, about 80% to about 90%, about 45% to about 95%, about 55% to about 95%, about 65% to about 95%, about 75% to about 95%, about 85% to about 95%, about 50% to about 80%, about 50% to about 70%, about 50% to about 60%, about 60% to about 80%, about 60% to about 70%, about 70% to about 80%, about 45% to about 85%, about 45% to about 75%, about 45% to about 65%, about 55% to about 85%, about 55% to about 75%, about 55% to about 65%, about 55% to about 60%, about 65% to about 85%, about 65% to about 75%, about 75% to about 85%, or about 80% to about 85% of the total weight of the dosage form. In some embodiments of the solid oral dosage forms disclosed herein, the filler is present in an amount of about 1% to about 95%, or about 50% to about 90% of the total weight of the dosage form. In some embodiments of the solid oral dosage forms disclosed herein, the filler is present in an amount of about 50% to about 90% of the total weight of the dosage form. In some embodiments, the filler is present in an amount of about 55% to about 85% of the total weight of the dosage form.

In some embodiments of the solid oral dosage forms disclosed herein, the filler is present in an amount of about 81% to about 83% of the total weight of the dosage form. In some embodiments of the solid oral dosage forms disclosed herein, the filler is present in an amount of about 82% of the total weight of the dosage form.

In some embodiments of the solid oral dosage forms disclosed herein, the filler is present in an amount of about 58% to about 60% of the total weight of the dosage form. In some embodiments of the solid oral dosage forms disclosed herein, the filler is present in an amount of about 59% of the total weight of the dosage form.

In some embodiments, the solid oral dosage form comprises the filler in an amount of about 50 mg to about 1000 mg. In some embodiments, the solid oral dosage form comprises the filler in an amount of about 50 mg to about 950 mg, about 50 mg to about 900 mg, about 50 mg to about 850 mg, about 50 mg to about 800 mg, about 50 mg to about 750 mg, about 50 mg to about 700 mg, about 50 mg to about 650 mg, about 50 mg to about 600 mg, about 50 mg to about 550 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 350 mg to about 450 mg, about 350 mg to about 400 mg, about 400 mg to about 500 mg, about 400 mg to about 450 mg, or about 450 mg to about 500 mg.

In some embodiments, the solid oral dosage form comprises the filler in an amount of about 60 mg to about 1000 mg. In some embodiments, the solid oral dosage form comprises the filler in an amount of about 90 mg to about 900 mg. In some embodiments, the solid oral dosage form comprises the filler in an amount of about 90 mg to about 500 mg. In some embodiments, the solid oral dosage form comprises the filler in an amount of about 111 mg. In some embodiments, the solid oral dosage form comprises the filler in an amount of about 99 mg. In some embodiments, the solid oral dosage form comprises the filler in an amount of about 493 mg. In some embodiments, the solid oral dosage form comprises the filler in an amount of about 641 mg. In some embodiments, the solid oral dosage form comprises the filler in an amount of about 887 mg.

In some embodiments of the pharmaceutical compositions disclosed herein, the pharmaceutical composition comprises lactose (e.g., lactose monohydrate). In some embodiments, the pharmaceutical composition comprises about 20 wt. % to about 50 wt. %, or about 25 wt. % to about 45 wt. % of the lactose. In some embodiments, the pharmaceutical composition comprises about 20 wt. % to about 50 wt. % lactose. In some embodiments, the pharmaceutical composition comprises about 25 wt. % to about 45 wt. % lactose. In some embodiments, the pharmaceutical composition comprises about 35 wt. % to about 45 wt. % lactose. In some embodiments, the pharmaceutical composition comprises about 41 wt. % lactose. In some embodiments, the pharmaceutical composition comprises about 25 wt. % to about 35 wt. % lactose. In some embodiments, the pharmaceutical composition comprises about 30 wt. % lactose.

In some embodiments of the solid oral dosage forms disclosed herein, the filler comprises lactose (e.g., lactose monohydrate). In some embodiments, the lactose is present in an amount of about 20% to about 50%, or about 25% to about 45% of the total weight of the dosage form. In some embodiments, the lactose is present in an amount of about 20% to about 50% of the total weight of the dosage form. In some embodiments, the lactose is present in an amount of about 25% to about 45% of the total weight of the dosage form.

In some embodiments, the oral dosage form comprises about 20 wt. % to about 50 wt. % of the lactose, wherein the lactose is lactose monohydrate. In some embodiments, the oral dosage form comprises about 25 wt. % to about 45 wt. % of the lactose, wherein the lactose is lactose monohydrate.

In some embodiments, the lactose is present in an amount of about 25% to about 35% of the total weight of the dosage form. In some embodiments, the lactose is present in an amount of about 29% to about 31% of the total weight of the dosage form. In some embodiments, the lactose is present in an amount of about 30% of the total weight of the dosage form.

In some embodiments, the oral dosage form comprises about 25 wt. % to about 35 wt. % of the lactose, wherein the lactose is lactose monohydrate. In some embodiments, the oral dosage form comprises about 30 wt. % of the lactose, wherein the lactose is lactose monohydrate.

In some embodiments, the lactose is present in an amount of about 35% to about 45% of the total weight of the dosage form. In some embodiments, the lactose is present in an amount of about 40% to about 42% of the total weight of the dosage form. In some embodiments, the lactose is present in an amount of about 41% of the total weight of the dosage form.

In some embodiments, the oral dosage form comprises about 35 wt. % to about 45 wt. % of the lactose, wherein the lactose is lactose monohydrate. In some embodiments, the oral dosage form comprises about 41 wt. % of the lactose, wherein the lactose is lactose monohydrate.

In some embodiments, the solid oral dosage form comprises the lactose (e.g., lactose monohydrate) in an amount of about 1 mg to about 1000 mg. In some embodiments, the solid oral dosage form comprises the lactose monohydrate in an amount of about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 10 mg to about 60 mg, about 50 mg to about 500 mg, about 50 mg to about 300 mg, about 50 mg to about 250 mg, about 45 mg to about 250 mg, about 50 mg to about 200 mg, about 50 mg to about 150 mg, about 100 mg to about 500 mg, about 100 mg to about 300 mg, about 100 mg to about 250 mg, about 100 mg to about 200 mg, about 200 mg to about 500 mg, about 200 mg to about 450 mg, about 200 mg to about 400 mg, about 200 mg to about 350 mg, about 200 mg to about 300 mg, or about 200 mg to about 250 mg.

In some embodiments, the solid oral dosage form comprises lactose in an amount of about 10 mg to about 500 mg. In some embodiments, the solid oral dosage form comprises lactose in an amount of about 30 mg to about 500 mg. In some embodiments, the solid oral dosage form comprises lactose in an amount of about 10 mg to about 450 mg. In some embodiments, the solid oral dosage form comprises lactose in an amount of about 10 mg to about 250 mg. In some embodiments, the solid oral dosage form comprises lactose in an amount of about 40 mg to about 250 mg. In some embodiments, the solid oral dosage form comprises lactose in an amount of about 45 mg to about 450 mg. In some embodiments, the solid oral dosage form comprises lactose in an amount of about 45 mg to about 250 mg. In some embodiments, the solid oral dosage form comprises lactose in an amount of about 55 mg. In some embodiments, the solid oral dosage form comprises lactose in an amount of about 49 mg. In some embodiments, the solid oral dosage form comprises lactose in an amount of about 246 mg. In some embodiments, the solid oral dosage form comprises lactose in an amount of about 320 mg. In some embodiments, the solid oral dosage form comprises lactose in an amount of about 444 mg.

In some embodiments of the pharmaceutical compositions disclosed herein, the pharmaceutical composition comprises microcrystalline cellulose. In some embodiments, the pharmaceutical composition comprises about 20 wt. % to about 50 wt. %, or about 25 wt. % to about 45 wt. % of the microcrystalline cellulose. In some embodiments, the pharmaceutical composition comprises about 20 wt. % to about 50 wt. % microcrystalline cellulose. In some embodiments, the pharmaceutical composition comprises about 25 wt. % to about 45 wt. % microcrystalline cellulose. In some embodiments, the pharmaceutical composition comprises about 35 wt. % to about 45 wt. % microcrystalline cellulose. In some embodiments, the pharmaceutical composition comprises about 41 wt. % microcrystalline cellulose. In some embodiments, the pharmaceutical composition comprises about 25 wt. % to about 35 wt. % microcrystalline cellulose. In some embodiments, the pharmaceutical composition comprises about 30 wt. % microcrystalline cellulose.

In some embodiments of the solid oral dosage forms disclosed herein, the filler comprises microcrystalline cellulose. In some embodiments, the microcrystalline cellulose is present in an amount of about 20% to about 50%, or about 25% to about 45% of the total weight of the dosage form. In some embodiments, the microcrystalline cellulose is present in an amount of about 20% to about 50% of the total weight of the dosage form. In some embodiments, the microcrystalline cellulose is present in an amount of about 25% to about 45% of the total weight of the dosage form.

In some embodiments, the microcrystalline cellulose is present in an amount of about 25% to about 35% of the total weight of the dosage form. In some embodiments, the microcrystalline cellulose is present in an amount of about 29% to about 31% of the total weight of the dosage form. In some embodiments, the microcrystalline cellulose is present in an amount of about 30% of the total weight of the dosage form.

In some embodiments, the microcrystalline cellulose is present in an amount of about 35% to about 45% of the total weight of the dosage form. In some embodiments, the microcrystalline cellulose is present in an amount of about 40% to about 42% of the total weight of the dosage form. In some embodiments, the microcrystalline cellulose is present in an amount of about 41% of the total weight of the dosage form.

In some embodiments, the solid oral dosage form comprises the microcrystalline cellulose in an amount of about 1 mg to about 1000 mg. In some embodiments, the solid oral dosage form comprises microcrystalline cellulose in an amount of about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 10 mg to about 60 mg, about 50 mg to about 500 mg, about 50 mg to about 300 mg, about 50 mg to about 250 mg, about 45 mg to about 250 mg, about 50 mg to about 200 mg, about 50 mg to about 150 mg, about 100 mg to about 500 mg, about 100 mg to about 300 mg, about 100 mg to about 250 mg, about 100 mg to about 200 mg, about 200 mg to about 500 mg, about 200 mg to about 450 mg, about 200 mg to about 400 mg, about 200 mg to about 350 mg, about 200 mg to about 300 mg, or about 200 mg to about 250 mg. In some embodiments, the solid oral dosage form comprises microcrystalline cellulose in an amount of about 10 mg to about 250 mg.

In some embodiments, the solid oral dosage form comprises microcrystalline cellulose in an amount of about 10 mg to about 500 mg. In some embodiments, the solid oral dosage form comprises microcrystalline cellulose in an amount of about 30 mg to about 500 mg. In some embodiments, the solid oral dosage form comprises microcrystalline cellulose in an amount of about 45 mg to about 450 mg. In some embodiments, the solid oral dosage form comprises microcrystalline cellulose in an amount of about 45 mg to about 250 mg. In some embodiments, the solid oral dosage form comprises microcrystalline cellulose in an amount of about 55 mg. In some embodiments, the solid oral dosage form comprises microcrystalline cellulose an amount of about 49 mg. In some embodiments, the solid oral dosage form comprises microcrystalline cellulose in an amount of about 246 mg. In some embodiments, the solid oral dosage form comprises microcrystalline cellulose in an amount of about 320 mg. In some embodiments, the solid oral dosage form comprises microcrystalline cellulose in an amount of about 444 mg.

In some embodiments, the filler comprises a mixture of lactose and microcrystalline cellulose, wherein a weight ratio of lactose to microcrystalline cellulose is about 1:4 to about 4:1, about 1:3 to about 3:1, about 2:3 to about 3:2, or about 1:1. In some embodiments, the filler comprises a mixture of lactose and microcrystalline cellulose, wherein a weight ratio of lactose to microcrystalline cellulose is about 2:3 to about 3:2. In some embodiments, the filler comprises a mixture of lactose and microcrystalline cellulose, wherein a weight ratio of lactose to microcrystalline cellulose is about 1:4, about 4:1, about 1:3, 3:1, about 2:3, about 3:2, or about 1:1. In some embodiments, the filler comprises a mixture of lactose and microcrystalline cellulose, wherein a weight ratio of lactose to microcrystalline cellulose is about 1:1.

In some embodiments, the oral dosage form comprises the compound of Formula I in an amount of about 5 mg to about 500 mg, and the filler in an amount of about 60 mg to about 1000 mg. In some embodiments, the oral dosage form comprises the compound of Formula I in an amount of about 10 mg to about 450 mg, and the filler in an amount of about 90 mg to about 900 mg. In some embodiments, the oral dosage form comprises the compound of Formula I in an amount of about 10 mg to about 250 mg, and the filler in an amount of about 90 mg to about 500 mg. In some embodiments, the oral dosage form comprises the compound of Formula I in an amount of about 10 mg, and the filler in an amount of about 111 mg. In some embodiments, the oral dosage form comprises the compound of Formula I in an amount of about 50 mg, and the filler in an amount of about 99 mg. In some embodiments, the oral dosage form comprises the compound of Formula I in an amount of about 250 mg, and the filler in an amount of about 493 mg. In some embodiments, the oral dosage form comprises the compound of Formula I in an amount of about 325 mg, and the filler in an amount of about 641 mg. In some embodiments, the oral dosage form comprises the compound of Formula I in an amount of about 450 mg, and the filler in an amount of about 887 mg. In some embodiments, the solid dosage form is a tablet.

In some embodiments, the oral dosage form comprises the compound of Formula I in an amount of about 5 mg to about 500 mg, lactose in an amount of about 30 mg to about 500 mg, and microcrystalline cellulose in an amount of about 30 mg to about 500 mg. In some embodiments, the oral dosage form comprises the compound of Formula I in an amount of about 10 mg to about 450 mg, lactose in an amount of about 45 mg to about 450 mg, and microcrystalline cellulose in an amount of about 45 mg to about 450 mg. In some embodiments, the oral dosage form comprises the compound of Formula I in an amount of about 10 mg to about 250 mg, lactose in an amount of about 45 mg to about 250 mg, and microcrystalline cellulose in an amount of about 45 mg to about 250 mg. In some embodiments, the oral dosage form comprises the compound of Formula I in an amount of about 10 mg, lactose in an amount of about 55 mg, and microcrystalline cellulose in an amount of about 55 mg. In some embodiments, the oral dosage form comprises the compound of Formula I in an amount of about 50 mg, lactose in an amount of about 49 mg, and microcrystalline cellulose in an amount of about 49 mg. In some embodiments, the oral dosage form comprises the compound of Formula I in an amount of about 250 mg, lactose in an amount of about 246 mg, and microcrystalline cellulose in an amount of about 246 mg. In some embodiments, the oral dosage form comprises the compound of Formula I in an amount of about 325 mg, lactose in an amount of about 320 mg, and microcrystalline cellulose in an amount of about 320 mg. In some embodiments, the oral dosage form comprises the compound of Formula I in an amount of about 450 mg, lactose in an amount of about 444 mg, and microcrystalline cellulose in an amount of about 444 mg. In some embodiments, the solid dosage form is a tablet.

### Disintegrant

In some embodiments of the pharmaceutical compositions (e.g., solid oral dosage forms) disclosed herein, the one or more excipients comprise a disintegrant (i.e., a disintegrating agent). Examples of disintegrating agents that can be used in the pharmaceutical compositions (e.g., solid oral dosage forms) described herein include, but are not limited to, starches (e.g., maize starch), pre-gelatinized starch, hydroxypropyl starch, celluloses (e.g., microcrystalline cellulose and low substituted hydroxypropyl cellulose), cross-linked PVP (crospovidone), sodium starch glycolate, croscarmellose sodium, and alginic acid. In some embodiments, the disintegrant comprises starch, pregelatinized starch, hydroxypropyl starch, celluloses, crospovidone, sodium starch glycolate, croscarmellose sodium, or a combination thereof. In some embodiments, the disintegrant comprises pregelatinized starch. In some embodiments, the disintegrant comprises croscarmellose sodium. In some embodiments, the disintegrant comprises crospovidone.

In some embodiments, the filler comprises lactose and microcrystalline cellulose, and the disintegrant comprises croscarmellose sodium.

In some embodiments of the pharmaceutical compositions disclosed herein, the pharmaceutical composition comprises about 1 wt. % to about 50 wt. %, about 1 wt. % to about 20 wt. %, about 1 wt. % to about 15 wt. %, about 1 wt. % to about 10 wt. %, about 5 wt. % to about 15 wt. %, or about 5 wt. % to about 8 wt. % of the disintegrant. In some embodiments, the pharmaceutical composition comprises about 1 wt. % to about 10 wt. % of a disintegrant.

In some embodiments of the solid oral dosage forms disclosed herein, the disintegrant is present in an amount of about 1% to about 50%, about 1% to about 20%, about 1% to about 15%, about 1% to about 10%, about 5% to about 15%, or about 5% to about 8% of the total weight of the dosage form (i.e., wt. %). In some embodiments, the disintegrant is present in an amount of about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% of the total weight of the dosage form.

In some embodiments of the solid oral dosage forms disclosed herein, the disintegrant is present in an amount of about 1% to about 10% of the total weight of the dosage form. In some embodiments, the disintegrant is present in an amount of about 3% to about 7% of the total weight of the dosage form. In some embodiments, the disintegrant is present in an amount of about 5% of the total weight of the dosage form.

In some embodiments, the solid oral dosage form comprises the disintegrant in an amount of about 1 mg to about 200 mg. In some embodiments, the solid oral dosage form comprises the disintegrant in an amount of about 5 mg to about 150 mg, 5 mg to about 140 mg, 5 mg to about 130 mg, 5 mg to about 120 mg, 5 mg to about 111 mg, 5 mg to about 100 mg, 5 mg to about 90 mg, 5 mg to about 80 mg, 5 mg to about 70 mg, 5 mg to about 60 mg, 5 mg to about 50 mg, 5 mg to about 45 mg, 5 mg to about 40 mg, about 5 mg to about 30 mg, about 5 mg to about 20 mg, about 5 mg to about 10 mg, about 10 mg to about 50 mg, about 10 mg to about 40 mg, about 10 mg to about 30 mg, about 10 mg to about 20 mg, about 20 mg to about 50 mg, about 30 mg to about 50 mg, about 20 mg to about 30 mg, about 30 mg to about 40 mg, or about 40 mg to about 50 mg.

In some embodiments, the solid oral dosage form comprises the disintegrant in an amount of about 1 mg to about 100 mg. In some embodiments, the solid oral dosage form comprises the disintegrant in an amount of about 5 mg to about 80 mg. In some embodiments, the solid oral dosage form comprises the disintegrant in an amount of about 5 mg to about 45 mg. In some embodiments, the solid oral dosage form comprises the disintegrant in an amount of about 7 mg. In some embodiments, the solid oral dosage form comprises the disintegrant in an amount of about 8 mg. In some embodiments, the solid oral dosage form comprises the disintegrant in an amount of about 42 mg. In some embodiments, the solid oral dosage form comprises the disintegrant in an amount of about 54 mg. In some embodiments, the solid oral dosage form comprises the disintegrant in an amount of about 75 mg.

In some embodiments of the pharmaceutical compositions disclosed herein, the disintegrant comprises croscarmellose sodium. In some embodiments, the pharmaceutical composition comprises about 1 wt. % to about 50 wt. %, about 1 wt. % to about 20 wt. %, about 1 wt. % to about 15 wt. %, about 1 wt. % to about 10 wt. %, about 5 wt. % to about 15 wt. %, or about 5 wt. % to about 8 wt. % croscarmellose sodium. In some embodiments, the pharmaceutical composition comprises about 1 wt. % to about 10 wt. % croscarmellose sodium. In some embodiments, the pharmaceutical composition comprises about 3 wt. % to about 7 wt. % croscarmellose sodium. In some embodiments, the pharmaceutical composition comprises about 5 wt. % croscarmellose sodium.

In some embodiments of the solid oral dosage forms disclosed herein, the disintegrant comprises croscarmellose sodium. In some embodiments, the solid oral dosage form comprises about 1 wt. % to about 50 wt. %, about 1 wt. % to about 20 wt. %, about 1 wt. % to about 15 wt. %, about 1 wt. % to about 10 wt. %, about 5 wt. % to about 15 wt. %, or about 5 wt. % to about 8 wt. % croscarmellose sodium. In some embodiments, the solid oral dosage form comprises about 1 wt. % to about 10 wt. % croscarmellose sodium. In some embodiments, the solid oral dosage form comprises about 3 wt. % to about 7 wt. % croscarmellose sodium. In some embodiments, the solid oral dosage form comprises about 5 wt. % croscarmellose sodium.

In some embodiments, the solid oral dosage form comprises croscarmellose sodium in an amount of about 1 mg to about 200 mg. In some embodiments, the solid oral dosage form comprises croscarmellose sodium in an amount of about 1 mg to about 100 mg. In some embodiments, the solid oral dosage form comprises croscarmellose sodium in an amount of about 5 mg to about 80 mg. In some embodiments, the solid oral dosage form comprises croscarmellose sodium in an amount of about 5 mg to about 45 mg. In some embodiments, the solid oral dosage form comprises croscarmellose sodium in an amount of about 7 mg. In some embodiments, the solid oral dosage form comprises croscarmellose sodium in an amount of about 8 mg. In some embodiments, the solid oral dosage form comprises croscarmellose sodium in an amount of about 42 mg. In some embodiments, the solid oral dosage form comprises croscarmellose sodium in an amount of about 54 mg. In some embodiments, the solid oral dosage form comprises croscarmellose sodium in an amount of about 75 mg.

In some embodiments, the solid oral dosage form comprises the compound of Formula I in an amount of about 5 mg to about 500 mg, and the disintegrant in an amount of about 1 mg to about 100 mg. In some embodiments, the solid oral dosage form comprises the compound of Formula I in an amount of about 10 mg to about 450 mg, and the disintegrant in an amount of about 5 mg to about 80 mg. In some embodiments, the solid oral dosage form comprises the compound of Formula I in an amount of about 10 mg to about 250 mg, and the disintegrant in an amount of about 5 mg to about 45 mg. In some embodiments, the solid oral dosage form comprises the compound of Formula I in an amount of about 10 mg and the disintegrant in an amount of about 7 mg. In some embodiments, the solid oral dosage form comprises the compound of Formula I in an amount of about 50 mg and the disintegrant in an amount of about 8 mg. In some embodiments, the solid oral dosage form comprises the compound of Formula I in an amount of about 250 mg and the disintegrant in an amount of about 42 mg. In some embodiments, the solid oral dosage form comprises the compound of Formula I in an amount of about 325 mg and the disintegrant in an amount of about 54 mg. In some embodiments, the solid oral dosage form comprises the compound of Formula I in an amount of about 450 mg and the disintegrant in an amount of about 75 mg. In some embodiments, the solid oral dosage form is a tablet.

### Lubricant

In some embodiments of the pharmaceutical compositions (e.g., solid oral dosage forms) disclosed herein, the one or more excipients comprise a lubricant. Examples of lubricants that can be used in the pharmaceutical compositions (e.g., solid oral dosage forms) disclosed herein include, but are not limited to, magnesium stearate, calcium stearate, stearic acid, hydrogenated vegetable oil, glyceryl palmitostearate, glyceryl behenate, sodium stearyl fumarate, colloidal silicon dioxide, carnauba wax, polyethylene glycol, and talc powder. In some embodiments, the lubricant comprises stearic acid, sodium stearyl fumarate, magnesium stearate, or a combination thereof. In some embodiments, the lubricant comprises magnesium stearate.

In some embodiments, the filler comprises lactose and microcrystalline cellulose, the disintegrant comprises croscarmellose sodium, and the lubricant comprises magnesium stearate.

In some embodiments of the pharmaceutical compositions disclosed herein, the pharmaceutical composition comprises about 0.01 wt. % to about 10 wt. %, about 0.1 wt. % to about 5 wt. %, or about 0.1 wt. % to about 2 wt. % of the lubricant. In some embodiments, the pharmaceutical composition comprises about 0.1 wt. % to about 5 wt. % of a lubricant.

In some embodiments of the solid oral dosage forms disclosed herein, the lubricant is present in an amount of about 0.01% to about 10%, about 0.1% to about 5%, or about 0.1% to about 2% of the total weight of the dosage form (i.e., wt. %). In some embodiments, the lubricant is present in an amount of about 0.01% to about 10% of the total weight of the dosage form. In some embodiments, the lubricant is present in an amount of about 0.1% to about 5% of the total weight of the dosage form. In some embodiments, the lubricant is present in an amount of about 0.1% to about 2% of the total weight of the dosage form. In some embodiments, the lubricant is present in an amount of about 1% to about 2% of the total weight of the dosage form. In some embodiments, the lubricant is present in an amount of about 1.5% of the total weight of the dosage form.

In some embodiments, the solid oral dosage form comprises less than about 100 mg of lubricant. For example, the solid oral dosage form comprises less than about 90 mg, less than about 80 mg, less than about 70 mg, less than about 60 mg, less than about 50 mg, less than about 40 mg, less than about 30 mg, less than about 20 mg, less than about 15 mg, or less than about 10 mg of lubricant. In some embodiments, the solid oral dosage form comprises the lubricant in an amount of about 0.1 mg to about 100 mg, about 0.5 mg to about 50 mg, about 0.5 mg to about 40 mg, about 0.5 mg to about 30 mg, about 0.5 mg to about 20 mg, about 0.5 mg to about 15 mg, about 0.5 mg to about 10 mg, about 1 mg to about 15 mg, about 1.5 mg to about 12.5 mg, or about 2 mg to about 12 mg.

In some embodiments, the solid oral dosage form comprises the lubricant in an amount of about 0.5 mg to about 30 mg. In some embodiments, the solid oral dosage form comprises the lubricant in an amount of about 0.5 mg to about 25 mg. In some embodiments, the solid oral dosage form comprises the lubricant in an amount of about 0.5 mg to about 20 mg. In some embodiments, the solid oral dosage form comprises the lubricant in an amount of about 0.5 mg to about 10 mg. In some embodiments, the solid oral dosage form comprises the lubricant in an amount of about 2 mg to about 23 mg. In some embodiments, the solid oral dosage form comprises the lubricant in an amount of about 2 mg to about 12 mg. In some embodiments, the solid oral dosage form comprises the lubricant in an amount of about 2 mg. In some embodiments, the solid oral dosage form comprises the lubricant in an amount of about 2.5 mg. In some embodiments, the solid oral dosage form comprises the lubricant in an amount of about 12 mg. In some embodiments, the solid oral dosage form comprises the lubricant in an amount of about 16 mg. In some embodiments, the solid oral dosage form comprises the lubricant in an amount of about 22 mg.

In some embodiments, the pharmaceutical compositions disclosed herein comprise magnesium stearate. In some embodiments, the pharmaceutical composition comprises about 0.1 wt. % to about 2 wt. % magnesium stearate. In some embodiments, the pharmaceutical composition comprises about 1 wt. % to about 2 wt. % magnesium stearate. In some embodiments, pharmaceutical composition comprises about 1.5 wt. % magnesium stearate.

In some embodiments, the solid oral dosage forms disclosed herein comprises magnesium stearate. In some embodiments, the oral dosage form comprises about 0.1 wt. % to about 2 wt. % magnesium stearate. In some embodiments, the oral dosage form comprises about 1 wt. % to about 2 wt. % magnesium stearate. In some embodiments, solid oral dosage form comprises about 1.5 wt. % magnesium stearate. In some embodiments, the solid oral dosage form comprises magnesium stearate in an amount of about 0.1 mg to about 100 mg, about 0.5 mg to about 50 mg, about 0.5 mg to about 40 mg, about 0.5 mg to about 30 mg, about 0.5 mg to about 20 mg, about 0.5 mg to about 15 mg, about 0.5 mg to about 10 mg, about 1 mg to about 15 mg, about 1.5 mg to about 12.5 mg, or about 2 mg to about 12 mg.

In some embodiments, the solid oral dosage form comprises magnesium stearate in an amount of about 0.5 mg to about 30 mg. In some embodiments, the solid oral dosage form comprises magnesium stearate in an amount of about 0.5 mg to about 25 mg. In some embodiments, the solid oral dosage form comprises magnesium stearate in an amount of about 0.5 mg to about 20 mg. In some embodiments, the solid oral dosage form comprises magnesium stearate in an amount of about 0.5 mg to about 10 mg. In some embodiments, the solid oral dosage form comprises magnesium stearate in an amount of about 2 mg to about 23 mg. In some embodiments, the solid oral dosage form comprises magnesium stearate in an amount of about 2 mg to about 12 mg. In some embodiments, the solid oral dosage form comprises magnesium stearate in an amount of about 2 mg. In some embodiments, the solid oral dosage form comprises magnesium stearate in an amount of about 2.5 mg. In some embodiments, the solid oral dosage form comprises magnesium stearate in an amount of about 12 mg. In some embodiments, the solid oral dosage form comprises magnesium stearate in an amount of about 16 mg. In some embodiments, the solid oral dosage form comprises magnesium stearate in an amount of about 22 mg.

In some embodiments, the solid oral dosage form comprises the compound of Formula I in an amount of about 5 mg to about 500 mg and the lubricant in an amount of about 0.5 mg to about 30 mg. In some embodiments, the solid oral dosage form comprises the compound of Formula I in an amount of about 10 mg to about 450 mg and the lubricant in an amount of about 0.5 mg to about 25 mg. In some embodiments, the solid oral dosage form comprises the compound of Formula I in an amount of about 10 mg to about 250 mg and the lubricant in an amount of about 0.5 mg to about 10 mg. In some embodiments, the solid oral dosage form comprises the compound of Formula I in an amount of about 10 mg and the lubricant in an amount of about 2 mg. In some embodiments, the solid oral dosage form comprises the compound of Formula I in an amount of about 50 mg and the lubricant in an amount of about 2.5 mg. In some embodiments, the solid oral dosage form comprises the compound of Formula I in an amount of about 250 mg and the lubricant in an amount of about 12 mg. In some embodiments, the solid oral dosage form comprises the compound of Formula I in an amount of about 325 mg and the lubricant in an amount of about 16 mg. In some embodiments, the solid oral dosage form comprises the compound of Formula I in an amount of about 450 mg and the lubricant in an amount of about 22 mg. In some embodiments, the solid oral dosage form is a tablet.

In some embodiments, the pharmaceutical compositions (e.g., solid oral dosage forms) disclosed herein comprise intragranular lubricant and extragranular lubricant. In some embodiments, the pharmaceutical composition comprises about 0.05 wt. % to about 1 wt. % intragranular lubricant, and about 0.05 wt. % to about 1 wt. % extragranular lubricant. In some embodiments, the pharmaceutical composition comprises about 0.75 wt. % intragranular lubricant, and about 0.75 wt. % extragranular lubricant.

### Binder

In some embodiments of the pharmaceutical compositions (e.g., solid oral dosage forms) disclosed herein, the one or more excipients comprise a binder. Examples of binders that can be used in the pharmaceutical compositions (e.g., solid oral dosage forms) disclosed herein include, but are not limited to, celluloses (e.g., microcrystalline cellulose, hydroxypropyl cellulose), starches, gelatin, and acacia. In some embodiments, binder comprises a cellulose, starch, gelatin, acacia, or a combination thereof. In some embodiments, binder comprises microcrystalline cellulose, hydroxypropyl cellulose, starch, gelatin, acacia, or a combination thereof. In some embodiments, the binder comprises a cellulose. In some embodiments, the binder comprises hydroxypropyl cellulose.

In some embodiments, the filler comprises lactose and microcrystalline cellulose, the disintegrant comprises croscarmellose sodium, the lubricant comprises magnesium stearate, and the binder comprises hydroxypropyl cellulose.

In some embodiments of the pharmaceutical compositions disclosed herein, the pharmaceutical composition comprises about 1 wt. % to about 10 wt. % of a binder. In some embodiments of the pharmaceutical compositions disclosed herein, the pharmaceutical composition comprises about 2 wt. % to about 6 wt. % of a binder.

In some embodiments of the solid oral dosage forms disclosed herein, the binder is present in an amount of about 1% to about 10% of the total weight of the dosage form (i.e., wt. %). In some embodiments, the binder is present in an amount of about 1% to about 9%, about 1% to about 8%, about 1% to about 7%, about 1% to about 6%, about 1% to about 5%, about 2% to about 9%, about 2% to about 8%, about 2% to about 6%, about 2% to about 5%, about 3% to about 9%, about 3% to about 8%, about 3% to about 7%, about 3% to about 6%, about 3% to about 5%, or about 4% to about 6% of the total weight of the dosage form. In some embodiments, the binder is present in an amount of about 2% to about 6% of the total weight of the dosage form. In some embodiments, the binder is present in an amount of about 4% of the total weight of the dosage form.

In some embodiments, the solid oral dosage form comprises the binder in an amount of about 0.1 mg to about 100 mg. In some embodiments, the solid oral dosage form comprises the binder in an amount of about 1 mg to about 50 mg, about 1 mg to about 40 mg, about 1 mg to about 30 mg, about 1 mg to about 20 mg, about 1 mg to about 15 mg, about 1 mg to about 10 mg, about 5 mg to about 10 mg, about 10 mg to about 50 mg, about 10 mg to about 40 mg, about 20 mg to about 50 mg, about 20 mg to about 40 mg, about 20 mg to about 30 mg, about 30 mg to about 50 mg, about 30 mg to about 40 mg, or about 30 mg to about 35 mg.

In some embodiments, the solid oral dosage form comprises the binder in an amount of about 1 mg to about 80 mg. In some embodiments, the solid oral dosage form comprises the binder in an amount of about 5 mg to about 60 mg. In some embodiments, the solid oral dosage form comprises the binder in an amount of about 5 mg to about 35 mg. In some embodiments, the solid oral dosage form comprises the binder in an amount of about 5 mg. In some embodiments, the solid oral dosage form comprises the binder in an amount of about 7 mg. In some embodiments, the solid oral dosage form comprises the binder in an amount of about 33 mg. In some embodiments, the solid oral dosage form comprises the binder in an amount of about 43 mg. In some embodiments, the solid oral dosage form comprises the binder in an amount of about 60 mg.

In some embodiments, the pharmaceutical compositions disclosed herein comprises hydroxypropyl cellulose. In some embodiments, the pharmaceutical composition comprises about 1 wt. % to about 10 wt. % hydroxypropyl cellulose. In some embodiments, the pharmaceutical composition comprises about 2 wt. % to about 6 wt. % hydroxypropyl cellulose. In some embodiments, the pharmaceutical composition comprises about 4 wt. % hydroxypropyl cellulose.

In some embodiments, the solid oral dosage forms disclosed herein comprises hydroxypropyl cellulose. In some embodiments, the oral dosage form comprises about 1 wt. % to about 10 wt. % hydroxypropyl cellulose. In some embodiments, the oral dosage form comprises about 2 wt. % to about 6 wt. % hydroxypropyl cellulose. In some embodiments, the oral dosage form comprises about 4 wt. % hydroxypropyl cellulose.

In some embodiments, the solid oral dosage form comprises hydroxypropyl cellulose in an amount of about 0.1 mg to about 100 mg. In some embodiments, the solid oral dosage form comprises hydroxypropyl cellulose in an amount of about 1 mg to about 80 mg. In some embodiments, the solid oral dosage form comprises hydroxypropyl cellulose in an amount of about 1 mg to about 60 mg. In some embodiments, the solid oral dosage form comprises hydroxypropyl cellulose in an amount of about 1 mg to about 50 mg. In some embodiments, the solid oral dosage form comprises hydroxypropyl cellulose in an amount of about 5 mg to about 60 mg. In some embodiments, the solid oral dosage form comprises hydroxypropyl cellulose in an amount of about 5 mg to about 35 mg. In some embodiments, the solid oral dosage form comprises hydroxypropyl cellulose in an amount of about 5 mg. In some embodiments, the solid oral dosage form comprises hydroxypropyl cellulose in an amount of about 7 mg. In some embodiments, the solid oral dosage form comprises hydroxypropyl cellulose in an amount of about 33 mg. In some embodiments, the solid oral dosage form comprises hydroxypropyl cellulose in an amount of about 43 mg. In some embodiments, the solid oral dosage form comprises hydroxypropyl cellulose in an amount of about 60 mg.

In some embodiments, the solid oral dosage form comprises the compound of Formula I in an amount of about 5 mg to about 500 mg and the binder in an amount of about 1 mg to about 80 mg. In some embodiments, the solid oral dosage form comprises the compound of Formula I in an amount of about 10 mg to about 450 mg and the binder in an amount of about 5 mg to about 60 mg. In some embodiments, the solid oral dosage form comprises the compound of Formula I in an amount of about 10 mg to about 250 mg and the binder in an amount of about 5 mg to about 35 mg. In some embodiments, the solid oral dosage form comprises the compound of Formula I in an amount of about 10 mg and the binder in an amount of about 5 mg. In some embodiments, the solid oral dosage form comprises the compound of Formula I in an amount of about 50 mg and the binder in an amount of about 7 mg. In some embodiments, the solid oral dosage form comprises the compound of Formula I in an amount of about 250 mg and the binder in an amount of about 33 mg. In some embodiments, the solid oral dosage form comprises the compound of Formula I in an amount of about 325 mg and the binder in an amount of about 43 mg. In some embodiments, the solid oral dosage form comprises the compound of Formula I in an amount of about 450 mg and the binder in an amount of about 60 mg. In some embodiments, the solid oral dosage form is a tablet.

In some embodiments, provided herein is a pharmaceutical composition comprising a compound of Formula I or a pharmaceutically acceptable salt thereof; a filler; a disintegrant; a binder; and a lubricant. In some embodiments, provided herein is a pharmaceutical composition comprising a compound of Formula I or a pharmaceutically acceptable salt thereof; a filler; a disintegrant; a binder; and a lubricant; wherein the compound of Formula I has crystalline Form I.

In some embodiments, the pharmaceutical composition comprises:
about 5 wt. % to about 35 wt. % of the compound of Formula I, or a pharmaceutically acceptable salt thereof;
about 50 wt. % to about 90 wt. % of the filler;
about 1 wt. % to about 10 wt. % of the disintegrant;
about 1 wt. % to about 10 wt. % of the binder; and
about 0.1 wt. % to about 5 wt. % of the lubricant.

In some embodiments, provided herein is a pharmaceutical composition comprising a compound of Formula I or a pharmaceutically acceptable salt thereof; lactose, microcrystalline cellulose, croscarmellose sodium, hydroxypropyl cellulose, and magnesium stearate. In some embodiments, provided herein is a pharmaceutical composition comprising a compound of Formula I or a pharmaceutically acceptable salt thereof; lactose, microcrystalline cellulose, croscarmellose sodium, hydroxypropyl cellulose, and magnesium stearate, wherein the compound of Formula I has crystalline Form I. In some embodiments, provided herein is a pharmaceutical composition comprising a compound of Formula I or a pharmaceutically acceptable salt thereof; lactose, microcrystalline cellulose, croscarmellose sodium, hydroxypropyl cellulose, and magnesium stearate; wherein the lactose is lactose monohydrate. In some embodiments, provided herein is a pharmaceutical composition comprising a compound of Formula I or a pharmaceutically acceptable salt thereof; lactose, microcrystalline cellulose, croscarmellose sodium, hydroxypropyl cellulose, and magnesium stearate, wherein a weight ratio of the lactose to the microcrystalline cellulose is about 1:1.

In some embodiments, the pharmaceutical composition comprises:
about 5 wt. % to about 35 wt. % of the compound of Formula I, or a pharmaceutically acceptable salt thereof;
about 20 wt. % to about 50 wt. % lactose;
about 20 wt. % to about 50 wt. % microcrystalline cellulose;
about 1 wt. % to about 10 wt. % croscarmellose sodium;
about 1 wt. % to about 10 wt. % hydroxypropyl cellulose; and
about 0.1 wt. % to about 2 wt. % magnesium stearate.

In some embodiments, the pharmaceutical composition comprises:
about 5 wt. % to about 35 wt. % of the compound of Formula I;
about 25 wt. % to about 45 wt. % lactose;
about 25 wt. % to about 45 wt. % microcrystalline cellulose;
about 3 wt. % to about 7 wt. % croscarmellose sodium;
about 2 wt. % to about 6 wt. % hydroxypropyl cellulose; and about 0.1 wt. % to about 2 wt. % magnesium stearate.

In some embodiments, the pharmaceutical composition comprises:
about 5 wt. % to about 10 wt. % of the compound of Formula I;
about 35 wt. % to about 45 wt. % lactose;
about 35 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

In some embodiments, the pharmaceutical composition comprises:
about 7.4 wt. % of the compound of Formula I;
about 41 wt. % lactose;
about 41 wt. % microcrystalline cellulose;
about 5 wt. % croscarmellose sodium;
about 4 wt. % hydroxypropyl cellulose; and
about 1.5 wt. % magnesium stearate.

In some embodiments, the pharmaceutical composition comprises:
about 28 wt. % to about 32 wt. % of the compound of Formula I;
about 25 wt. % to about 35 wt. % lactose;
about 25 wt. % to about 35 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

In some embodiments, the pharmaceutical composition comprises:
about 30 wt. % of the compound of Formula I;
about 30 wt. % lactose;
about 30 wt. % microcrystalline cellulose;
about 5 wt. % croscarmellose sodium;
about 4 wt. % hydroxypropyl cellulose; and
about 1.5 wt. % magnesium stearate.

In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; a filler; a disintegrant; a binder; and a lubricant. In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; a filler; a disintegrant; a binder; and a lubricant; wherein the compound of Formula I has crystalline Form I.

In some embodiments, the oral dosage form comprises:
about 5 wt. % to about 35 wt. % of the compound of Formula I, or a pharmaceutically acceptable salt thereof;
about 50 wt. % to about 90 wt. % of the filler;
about 1 wt. % to about 10 wt. % of the disintegrant;
about 1 wt. % to about 10 wt. % of the binder; and
about 0.1 wt. % to about 5 wt. % of the lubricant.

In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; lactose, microcrystalline cellulose, croscarmellose sodium, hydroxypropyl cellulose, and magnesium stearate. In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; lactose, microcrystalline cellulose, croscarmellose sodium, hydroxypropyl cellulose, and magnesium stearate; wherein the compound of Formula I has crystalline Form I. In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; lactose, microcrystalline cellulose, croscarmellose sodium, hydroxypropyl cellulose, and magnesium stearate; wherein the lactose is lactose monohydrate. In some embodiments, provided herein is a solid oral dosage form (e.g., a tablet) comprising a compound of Formula I or a pharmaceutically acceptable salt thereof, in an amount of about 5 mg to about 1400 mg, wherein the amount is based on the free acid form; lactose, microcrystalline cellulose, croscarmellose sodium, hydroxypropyl cellulose, and magnesium stearate; wherein a weight ratio of the lactose to the microcrystalline cellulose is about 1:1.

In some embodiments, the oral dosage form comprises:
about 5 wt. % to about 35 wt. % of the compound of Formula I, or a pharmaceutically acceptable salt thereof;
about 20 wt. % to about 50 wt. % lactose;
about 20 wt. % to about 50 wt. % microcrystalline cellulose;
about 1 wt. % to about 10 wt. % croscarmellose sodium;
about 1 wt. % to about 10 wt. % hydroxypropyl cellulose; and
about 0.1 wt. % to about 2 wt. % magnesium stearate.

In some embodiments, the oral dosage form comprises:
about 5 wt. % to about 35 wt. % of the compound of Formula I;
about 25 wt. % to about 45 wt. % lactose;
about 25 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

In some embodiments, the oral dosage form comprises:
about 5 wt. % to about 10 wt. % of the compound of Formula I;
about 35 wt. % to about 45 wt. % lactose;
about 35 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

In some embodiments, the oral dosage form comprises:
about 7.4 wt. % of the compound of Formula I;
about 41 wt. % lactose;
about 41 wt. % microcrystalline cellulose;
about 5 wt. % croscarmellose sodium;
about 4 wt. % hydroxypropyl cellulose; and
about 1.5 wt. % magnesium stearate.

In some embodiments, the oral dosage form comprises:
about 28 wt. % to about 32 wt. % of the compound of Formula I;
about 25 wt. % to about 35 wt. % lactose;
about 25 wt. % to about 35 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

In some embodiments, the oral dosage form comprises:
about 30 wt. % of the compound of Formula I;
about 30 wt. % lactose;
about 30 wt. % microcrystalline cellulose;
about 5 wt. % croscarmellose sodium;
about 4 wt. % hydroxypropyl cellulose; and
about 1.5 wt. % magnesium stearate.

In some embodiments, the oral dosage form comprises:

the compound of Formula I in an amount of about 5 mg to about 500 mg;
the filler in an amount of about 60 mg to about 1000 mg;
the disintegrant in an amount of about 1 mg to about 100 mg;
the binder in an amount of about 1 mg to about 80 mg; and
the lubricant in an amount of about 0.5 mg to about 30 mg.

In some embodiments, the oral dosage form comprises:
the compound of Formula I in an amount of about 5 mg to about 500 mg;
lactose in an amount of about 30 mg to about 500 mg;
microcrystalline cellulose in an amount of about 30 mg to about 500 mg;
croscarmellose sodium in an amount of about 1 mg to about 100 mg;
hydroxypropyl cellulose in an amount of about 1 mg to about 80 mg; and
magnesium stearate in an amount of about 0.5 mg to about 30 mg.

In some embodiments, the oral dosage form comprises:
the compound of Formula I in an amount of about 10 mg to about 450 mg;
the filler in an amount of about 90 mg to about 900 mg;
the disintegrant in an amount of about 5 mg to about 80 mg;
the binder in an amount of about 5 mg to about 60 mg; and
the lubricant in an amount of about 0.5 mg to about 25 mg.

In some embodiments, the oral dosage form comprises:
the compound of Formula I in an amount of about 10 mg to about 450 mg;
lactose in an amount of about 45 mg to about 450 mg;
microcrystalline cellulose in an amount of about 45 mg to about 450 mg;
croscarmellose sodium in an amount of about 5 mg to about 80 mg;
hydroxypropyl cellulose in an amount of about 5 mg to about 60 mg; and
magnesium stearate in an amount of about 0.5 mg to about 25 mg.

In some embodiments, the oral dosage form comprises:
the compound of Formula I in an amount of about 10 mg to about 250 mg;
the filler in an amount of about 90 mg to about 500 mg;
the disintegrant in an amount of about 5 mg to about 45 mg;
the binder in an amount of about 5 mg to about 35 mg; and
the lubricant in an amount of about 0.5 mg to about 20 mg.

In some embodiments, the oral dosage form comprises:
the compound of Formula I in an amount of about 10 mg to about 250 mg;
lactose in an amount of about 45 mg to about 250 mg;
microcrystalline cellulose in an amount of about 45 mg to about 250 mg;
croscarmellose sodium in an amount of about 5 mg to about 45 mg;
hydroxypropyl cellulose in an amount of about 5 mg to about 35 mg; and
magnesium stearate in an amount of about 0.5 mg to about 20 mg.

In some embodiments, the oral dosage form comprises:
the compound of Formula I in an amount of about 10 mg;
lactose in an amount of about 55 mg;
microcrystalline cellulose in an amount of about 55 mg;
croscarmellose sodium in an amount of about 7 mg;
hydroxypropyl cellulose in an amount of about 5 mg; and
magnesium stearate in an amount of about 2 mg.

In some embodiments, the oral dosage form comprises:
the compound of Formula I in an amount of about 50 mg;
lactose in an amount of about 49 mg;
microcrystalline cellulose in an amount of about 49 mg;
croscarmellose sodium in an amount of about 8 mg;
hydroxypropyl cellulose in an amount of about 7 mg; and
magnesium stearate in an amount of about 2.5 mg.

In some embodiments, the oral dosage form comprises:
the compound of Formula I in an amount of about 250 mg;
lactose in an amount of about 246 mg;
microcrystalline cellulose in an amount of about 246 mg;
croscarmellose sodium in an amount of about 42 mg;
hydroxypropyl cellulose in an amount of about 33 mg; and
magnesium stearate in an amount of about 12 mg.

In some embodiments, the oral dosage form comprises:
the compound of Formula I in an amount of about 325 mg;
lactose in an amount of about 320 mg;
microcrystalline cellulose in an amount of about 320 mg;
croscarmellose sodium in an amount of about 54 mg;
hydroxypropyl cellulose in an amount of about 43 mg; and
magnesium stearate in an amount of about 16 mg.

In some embodiments, the oral dosage form comprises:
the compound of Formula I in an amount of about 450 mg;
lactose in an amount of about 444 mg;
microcrystalline cellulose in an amount of about 444 mg;
croscarmellose sodium in an amount of about 75 mg;
hydroxypropyl cellulose in an amount of about 60 mg; and
magnesium stearate in an amount of about 22 mg.

### Other Excipients

Any other suitable excipients may be used with the pharmaceutical compositions disclosed herein. In some embodiments, the pharmaceutical compositions (e.g., solid oral dosage forms) described herein comprise a surfactant. In some embodiments, the surfactant comprises sodium lauryl sulfate, d-α-Tocopheryl polyethylene glycol 1000 succinate (TPGS), or a combination thereof.

In some embodiments, the pharmaceutical compositions (e.g., solid oral dosage forms) described herein comprise a pH modifier. In some embodiments, the pH modifier is an alkaline excipient. In some embodiments, the pH modifier is sodium carbonate, sodium bicarbonate, or a combination thereof. In some embodiments, the pH modifier is sodium bicarbonate.

In some embodiments, the pharmaceutical compositions (e.g., solid oral dosage forms) described herein comprise a glidant. In some embodiments, the glidant is colloidal silicon dioxide.

### Film Coat

In some embodiments, the solid oral dosage forms (e.g. tablets) provided herein are uncoated. In some embodiments, the solid dosage forms (e.g. tablets) provided herein are coated (e.g., they include a film coat). In some embodiments, the coated solid dosage forms (e.g. tablets) comprise a non-enteric coating. Example film coats can be comprised of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though in some embodiments the water soluble material included in the film coat of the embodiments disclosed herein comprises a single polymer material, in certain other embodiments it is formed using a mixture of more than one polymer.

Suitable film coats include, but are not limited to, polymeric film coatings such as those comprising polyvinyl alcohol e.g. ‘Opadry® II’ (which comprises part-hydrolysed PVA, titanium dioxide, macrogol 3350 (PEG) and talc, with optional coloring such as iron oxide (e.g., iron oxide red or iron oxide black) or indigo carmine or iron oxide yellow or FD&C yellow #6) and Opadry® II (which comprises polyethylene glycol/macrogol polyvinyl alcohol graft copolymer, talc, titanium dioxide, glyceryl mono and dicaprylocaprate (glyceryl monocaprylocaprate type I), polyvinyl alcohol, and with optional coloring such as iron oxide (e.g., iron oxide red or iron oxide black) or indigo carmine or iron oxide yellow or FD&C yellow #6). In some embodiments, the film coat comprises Opadry® II. In some embodiments, the film coat is beige. In some embodiments, the film coat comprises Opadry® II beige. In some embodiments, the film coat is beige. In some embodiments, the film coat comprises Opadry® II beige 85F570135. In some embodiments, the oral solid dosage form comprises from about 1% to about 5% of the film coat (based on the tablet core weight). In some embodiments, the oral solid dosage form comprises from about 2% to about 4% of the film coat (based on the tablet core weight). In some embodiments, the amount of the film coat is about 3% by weight (based on the tablet core weight).

Unless specifically stated otherwise, where the dosage form is coated, it is to be understood that a reference to % weight is calculated with respect to the tablet core weight.

The “tablet core weight” as calculated herein is the sum total weight of the compound of Formula I, or pharmaceutically acceptable salt thereof, and the one or more excipients in the tablet core. In some embodiments, the tablet core weight is the sum total weight of the compound of Formula I or pharmaceutically acceptable salt thereof, the filler, the disintegrant, the binder, and the lubricant. The core tablet weight does not include the weight of the film coat.

The “tablet weight” as calculated herein is the sum total weight of (i) the compound of Formula I or pharmaceutically acceptable salt thereof, and one or more excipients, and (ii) the film coat. In some embodiments, the tablet weight is the sum total weight of the compound of Formula I or pharmaceutically acceptable salt thereof, the filler, the disintegrant, the binder, the lubricant, and the film coat.

In some embodiments of the solid oral dosage forms disclosed herein, the dosage form is a tablet comprising a tablet core and a film coat. The “tablet core” refers to the compound of Formula I, or pharmaceutically acceptable salt thereof, and the one or more excipients in admixture with the compound of Formula I, and does not include the film coat. In some embodiments, the tablet core comprises intragranular excipients and extragranular excipients. In some embodiments, the tablet core comprises the compound of Formula I or pharmaceutically acceptable salt thereof, the filler, the disintegrant, the binder, and the lubricant.

In some embodiments of the solid oral dosage forms disclosed herein, the dosage form is a tablet comprising a tablet core and a film coat, wherein the tablet core comprises the compound of Formula I or pharmaceutically acceptable salt thereof, the filler, the disintegrant, the binder, and the lubricant.

In some embodiments, the solid dosage form comprises:

| Ingredient | % w/w |
|---|---|
| Compound of Formula I | 7.4% |
| Filler | 82.1% |
| Disintegrant | 5% |
| Binder | 4% |
| Lubricant | 1.5% |
| Film Coat | 3% |

In some embodiments, the solid dosage form is a tablet comprising:

| Ingredient | % w/w |
|---|---|
| Compound of Formula I | 30.1% |
| Filler | 59.4% |
| Disintegrant | 5% |
| Binder | 4% |
| Lubricant | 1.5% |

In some embodiments, the solid dosage form is a tablet comprising:

| Ingredient | % w/w |
|---|---|
| Compound of Formula I | 30.1% |
| Filler | 59.4% |
| Disintegrant | 5% |
| Binder | 4% |
| Lubricant | 1.5% |
| Film Coat | 3% |

In some embodiments, the solid dosage form is a tablet comprising:

| | Ingredient | % w/w | Amount (mg) | |
|---|---|---|---|---|
| | Compound of Formula I | 7.4% | 10 | mg |
| | Filler | 82.1% | 110.8 | mg |
| | Disintegrant | 5.0% | 6.8 | mg |
| | Binder | 4.0% | 5.4 | mg |
| | Lubricant | 1.5% | 2 | mg |
| Tablet core weight | | | 135 | mg |
| | Film Coat | 3% | 4 | mg |

| | | |
|---|---|---|
| Tablet weight | 139 | mg |

In some embodiments, the solid dosage form is a tablet comprising:

| | Ingredient | % w/w | Amount (mg) | |
|---|---|---|---|---|
| | Compound of Formula I | 30.1% | 50 | mg |
| | Filler | 59.4% | 98.6 | mg |
| | Disintegrant | 5.0% | 8.3 | mg |
| | Binder | 4.0% | 6.6 | mg |
| | Lubricant | 1.5% | 2.5 | mg |
| Tablet core weight | | | 166 | mg |
| | Film Coat | 3% | 5 | mg |

| | | |
|---|---|---|
| Tablet weight | 171 | mg |

In some embodiments, the solid dosage form is a tablet comprising:

| | Ingredient | % w/w | Amount (mg) | |
|---|---|---|---|---|
| | Compound of Formula I | 30.1% | 250 | mg |
| | Filler | 59.4% | 492.9 | mg |
| | Disintegrant | 5.0% | 41.5 | mg |
| | Binder | 4.0% | 33.2 | mg |
| | Lubricant | 1.5% | 12.4 | mg |
| Tablet core weight | | | 830 | mg |
| | Film Coat | 3% | 25 | mg |

| | | |
|---|---|---|
| Tablet weight | 855 | mg |

In some embodiments, the solid dosage form is a tablet comprising:

| Ingredient | % w/w | Amount (mg) | |
|---|---|---|---|
| Compound of Formula I | 30.1% | 325 | mg |
| Filler | 59.4% | 640.8 | mg |
| Disintegrant | 5.0% | 54.0 | mg |
| Binder | 4.0% | 43.2 | mg |
| Lubricant | 1.5% | 16.2 | mg |

| | | |
|---|---|---|
| Tablet core weight | 1079 | mg |

| | | |
|---|---|---|
| Film Coat | 3.0% | 32.4 |

| | | |
|---|---|---|
| Tablet weight | 1111 | mg |

In some embodiments, the solid dosage form is a tablet comprising:

| | Ingredient | % w/w | Amount (mg) | |
|---|---|---|---|---|
| | Compound of Formula I | 30.1% | 450 | mg |
| | Filler | 59.4% | 887.2 | mg |
| | Disintegrant | 5.0% | 74.7 | mg |
| | Binder | 4.0% | 59.8 | mg |
| | Lubricant | 1.5% | 22.4 | mg |
| Tablet core weight | | | 1494 | mg |
| | Film Coat | 3.0% | 44.8 | mg |

| | | |
|---|---|---|
| Tablet weight | 1539 | mg |

In some embodiments, the solid dosage form is a tablet comprising:

| | | |
|---|---|---|
| Ingredient | % w/w | Amount (mg) |

Intragranular

| | | | |
|---|---|---|---|
| Compound of Formula I | 7.4% | 10 | mg |
| Filler (lactose monohydrate and | 82.1% | 110.8 | mg |
| microcrystalline cellulose, 1:1 w/w) | | | |
| Disintegrant (croscarmellose sodium) | 5.0% | 6.8 | mg |
| Binder (hydroxypropyl cellulose) | 4.0% | 5.4 | mg |
| Lubricant (magnesium stearate) | 0.75% | 1 | mg |

Extragranular

| Lubricant (magnesium stearate) | 0.75% | 1 | mg |
|---|---|---|---|
| Tablet core weight | | 135 | mg |
| Film Coat | 3% | 4 | mg |

| | | |
|---|---|---|
| Tablet weight | 139 | mg |

In some embodiments, the solid dosage form comprises:

| | | |
|---|---|---|
| Ingredient | % w/w | Amount (mg) |

Intragranular

| | | | |
|---|---|---|---|
| Compound of Formula I | 30.1% | 50 | mg |
| Filler (lactose monohydrate and | 59.4% | 98.6 | mg |
| microcrystalline cellulose, 1:1 w/w) | | | |
| Disintegrant (croscarmellose sodium) | 5.0% | 8.3 | mg |
| Binder (hydroxypropyl cellulose) | 4.0% | 6.6 | mg |
| Lubricant (magnesium stearate) | 0.75% | 1.25 | mg |

Extragranular

| Lubricant (magnesium stearate) | 0.75% | 1.25 | mg |
|---|---|---|---|
| Tablet core weight | | 166 | mg |
| Film Coat | 3% | 5 | mg |

| | | |
|---|---|---|
| Tablet weight | 171 | mg |

In some embodiments, the solid dosage form comprises:

| | | |
|---|---|---|
| Ingredient | % w/w | Amount (mg) |

Intragranular

| | | | |
|---|---|---|---|
| Compound of Formula I | 30.1% | 250 | mg |
| Filler (lactose monohydrate and | 59.4% | 492.9 | mg |
| microcrystalline cellulose, 1:1 w/w) | | | |
| Disintegrant (croscarmellose sodium) | 5.0% | 41.5 | mg |
| Binder (hydroxypropyl cellulose) | 4.0% | 33.2 | mg |
| Lubricant (magnesium stearate) | 0.75% | 6.2 | mg |

Extragranular

| Lubricant (magnesium stearate) | 0.75% | 6.2 | mg |
|---|---|---|---|
| Tablet core weight | | 830 | mg |
| Film Coat | 3% | 25 | mg |

| | | |
|---|---|---|
| Tablet weight | 855 | mg |

In some embodiments, the solid dosage form comprises:

| | | |
|---|---|---|
| Ingredient | % w/w | Amount (mg) |

Intragranular

| | | | |
|---|---|---|---|
| Compound of Formula I | 30.1% | 325 | mg |
| Filler (lactose monohydrate and | 59.4% | 640.8 | mg |
| microcrystalline cellulose, 1:1 w/w) | | | |
| Disintegrant (croscarmellose sodium) | 5.0% | 54.0 | mg |
| Binder (hydroxypropyl cellulose) | 4.0% | 43.2 | mg |
| Lubricant (magnesium stearate) | 0.75% | 8.1 | mg |

Extragranular

| Lubricant (magnesium stearate) | 0.75% | 8.1 | mg |
|---|---|---|---|
| Tablet core weight | | 1079 | mg |
| Film Coat | 3.0% | 32.4 | mg |

| | | |
|---|---|---|
| Tablet weight | 1111 | mg |

In some embodiments, the solid dosage form comprises:

| | | |
|---|---|---|
| Ingredient | % w/w | Amount (mg) |

Intragranular

| | | |
|---|---|---|
| Compound of Formula I | 30.1% | 450 mg |
| Filler (lactose monohydrate and | 59.4% | 887.2 mg |
| microcrystalline cellulose, 1:1 w/w) | | |
| Disintegrant (croscarmellose sodium) | 5.0% | 74.7 mg |
| Binder (hydroxypropyl cellulose) | 4.0% | 59.8 mg |
| Lubricant (magnesium stearate) | 0.75% | 11.2 mg |

Extragranular

| | | |
|---|---|---|
| Lubricant (magnesium stearate) | 0.75% | 11.2 mg |
| Tablet core weight | | 1494 mg |
| Film Coat | 3.0% | 44.8 mg |
| Tablet weight | | 1539 mg |

In some embodiments, the solid dosage form comprises 10 mg of the compound of Formula I, lactose monohydrate, microcrystalline cellulose, croscarmellose sodium, hydroxypropyl cellulose, magnesium stearate, and a film coat. In some embodiments, the solid dosage form comprises 10 mg of the compound of Formula I, lactose monohydrate, microcrystalline cellulose, croscarmellose sodium, hydroxypropyl cellulose, magnesium stearate, and Opadry® II Beige.

In some embodiments, the solid dosage form comprises 50 mg of the compound of Formula I, lactose monohydrate, microcrystalline cellulose, croscarmellose sodium, hydroxypropyl cellulose, magnesium stearate, and a film coat. In some embodiments, the solid dosage form comprises 50 mg of the compound of Formula I, lactose monohydrate, microcrystalline cellulose, croscarmellose sodium, hydroxypropyl cellulose, magnesium stearate, and Opadry® II Beige.

In some embodiments, the solid dosage form comprises 250 mg of the compound of Formula I, lactose monohydrate, microcrystalline cellulose, croscarmellose sodium, hydroxypropyl cellulose, magnesium stearate, and a film coat. In some embodiments, the solid dosage form comprises 250 mg of the compound of Formula I, lactose monohydrate, microcrystalline cellulose, croscarmellose sodium, hydroxypropyl cellulose, magnesium stearate, and Opadry® II Beige.

In some embodiments, the solid dosage form comprises 325 mg of the compound of Formula I, lactose monohydrate, microcrystalline cellulose, croscarmellose sodium, hydroxypropyl cellulose, magnesium stearate, and a film coat. In some embodiments, the solid dosage form comprises 325 mg of the compound of Formula I, lactose monohydrate, microcrystalline cellulose, croscarmellose sodium, hydroxypropyl cellulose, magnesium stearate, and Opadry® II Beige.

In some embodiments, the solid dosage form comprises 450 mg of the compound of Formula I, lactose monohydrate, microcrystalline cellulose, croscarmellose sodium, hydroxypropyl cellulose, magnesium stearate, and a film coat. In some embodiments, the solid dosage form comprises 450 mg of the compound of Formula I, lactose monohydrate, microcrystalline cellulose, croscarmellose sodium, hydroxypropyl cellulose, magnesium stearate, and Opadry® II Beige.

In some embodiments of the solid oral dosage forms disclosed herein, the dosage form was prepared by wet granulation. In some embodiments of the solid oral dosage forms disclosed herein, the dosage form was prepared by dry granulation.

### IV. Methods of Use

Provided herein are methods for treating or preventing an HIV infection in a human, comprising orally administering to the human a therapeutically effective amount of a compound of Formula I, or a pharmaceutically acceptable salt thereof. The compound of Formula I, or a pharmaceutically acceptable salt thereof, can be formulated as a pharmaceutical composition described herein.

Accordingly, provided herein is a method for treating or preventing an HIV infection in a human, comprising orally administering to the human a therapeutically effective amount of any of the pharmaceutical compositions provided herein. In some embodiments, provided herein is a method for treating or preventing an HIV infection in a human, comprising orally administering to the human a solid oral dosage form or pharmaceutical composition provided herein. In some embodiments, the method comprises orally administering to the human a solid oral dosage form provided herein. In some embodiments, the method comprises orally administering to the human a tablet provided herein.

Also provided herein is the use of any of the pharmaceutical compositions provided herein for treating or preventing an HIV infection.

In some embodiments of the methods provided herein, the pharmaceutical composition (e.g., solid oral dosage form) is administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, 7, 8, 9, or once every 10 days. A single dose can also be administered once every 1 week, 2, 3, or once every 4 weeks. In certain embodiments, a single dose can be administered once every week.

In some embodiments, the pharmaceutical composition or solid oral dosage form disclosed herein is administered once every 1 day, 2 days, 3 days, 4 days, 5 days, 6 days, 7 days, 8 days, 9 days, or 10 days. In some embodiments, the pharmaceutical composition or solid oral dosage form disclosed herein is administered once every day (QD). In some embodiments, the pharmaceutical composition or solid oral dosage form disclosed herein is administered once every two days. In some embodiments, the pharmaceutical composition or solid oral dosage form disclosed herein is administered once every three days. In some embodiments, the pharmaceutical composition or solid oral dosage form disclosed herein is administered once every four days. In some embodiments, the pharmaceutical composition or solid oral dosage form disclosed herein is administered once every five days. In some embodiments, the pharmaceutical composition or solid oral dosage form disclosed herein is administered once every six days. In some embodiments, the pharmaceutical composition or solid oral dosage form disclosed herein is administered once every seven days. In some embodiments, the pharmaceutical composition or solid oral dosage form disclosed herein is administered once every eight days. In some embodiments, the pharmaceutical composition or solid oral dosage form disclosed herein is administered once every seven days. In some embodiments, the pharmaceutical composition or solid oral dosage form disclosed herein is administered once every nine days. In some embodiments, the pharmaceutical composition or solid oral dosage form disclosed herein is administered once every seven days. In some embodiments, the pharmaceutical composition or solid oral dosage form disclosed herein is administered once every ten days.

In some embodiments, the pharmaceutical composition or solid oral dosage form disclosed herein is administered once weekly (QW). In some embodiments, the pharmaceutical composition or solid oral dosage form disclosed herein is administered twice weekly. In some embodiments, the pharmaceutical composition or solid oral dosage form disclosed herein is administered once every two weeks (Q2W). In some embodiments, the pharmaceutical composition or solid oral dosage form disclosed herein is administered once every three weeks (Q3W).

In some embodiments, the method comprises administering a dose amount of from about 1 mg to about 2500 mg of the compound of Formula I, or a pharmaceutically acceptable salt thereof, wherein the dose amount is based on the free acid form. In some embodiments, the method comprises administering a dose amount of from about 10 mg to about 1500 mg of the compound of Formula I, or a pharmaceutically acceptable salt thereof, wherein the dose amount is based on the free acid form. In some embodiments, the method comprises administering a dose amount of from about 10 mg to about 1450 mg, about 10 mg to about 1400 mg, about 10 mg to about 1350 mg, about 10 mg to about 750 mg, about 10 mg to about 500 mg, about 10 mg to about 400 mg, about 10 mg to about 300 mg, about 10 mg to about 200 mg, about 10 mg to about 100 mg, about 10 mg to about 50 mg, about 10 mg to about 30 mg, about 50 mg to about 1000 mg, about 50 mg to about 800 mg, about 50 mg to about 600 mg, about 50 mg to about 400 mg, about 50 mg to about 300 mg, about 50 mg to about 100 mg, about 100 mg to about 1000 mg, about 100 mg to about 800 mg, about 100 mg to about 600 mg, about 100 mg to about 400 mg, about 100 mg to about 200 mg, about 200 mg to about 1000 mg, about 200 mg to about 800 mg, about 200 mg to about 700 mg, about 200 mg to about 600 mg, about 200 mg to about 400 mg, about 200 mg to about 300 mg, about 300 mg to about 1000 mg, about 300 mg to about 800 mg, about 300 mg to about 600 mg, about 300 mg to about 500 mg, about 300 mg to about 400 mg, about 400 mg to about 1000 mg, about 400 mg to about 800 mg, about 400 mg to about 700 mg, about 400 mg to about 600 mg, about 400 mg to about 500 mg, about 500 mg to about 1000 mg, about 500 mg to about 800 mg, about 500 mg to about 600 mg, about 600 mg to about 1000 mg, about 600 mg to about 900 mg, about 600 mg to about 800 mg, about 600 mg to about 700 mg, about 700 mg to about 1000 mg, about 700 mg to about 900 mg, about 700 mg to about 800 mg, about 800 mg to about 1000 mg, about 800 mg to about 900 mg, about 900 mg to about 1000 mg, about 200 mg to about 1500 mg, about 200 mg to about 1400 mg, about 400 mg to about 1300 mg, about 600 mg to about 1350 mg, about 800 mg to about 1200 mg, about 800 mg to about 1000 mg, about 1000 mg to about 1500 mg, about 1000 mg to about 1200 mg, or about 1200 mg to about 1400 mg of the compound of Formula I, or a pharmaceutically acceptable salt thereof, wherein the dose amount is based on the free acid form.

In some embodiments, the method comprises administering a dose amount of from about 5 mg to about 1350 mg of the compound of Formula I, or a pharmaceutically acceptable salt thereof, wherein the dose amount is based on the free acid form. In some embodiments, the method comprises administering a dose amount of from about 10 mg to about 1350 mg of the compound of Formula I, or a pharmaceutically acceptable salt thereof, wherein the dose amount is based on the free acid form. In some embodiments, the method comprises administering a dose amount of from about 50 mg to about 500 mg of the compound of Formula I, or a pharmaceutically acceptable salt thereof, wherein the dose amount is based on the free acid form. In some embodiments, the method comprises administering a dose amount of from about 10 mg to about 250 mg of the compound of Formula I, or a pharmaceutically acceptable salt thereof, wherein the dose amount is based on the free acid form. In some embodiments, the method comprises administering a dose amount of from about 200 mg to about 300 mg of the compound of Formula I, or a pharmaceutically acceptable salt thereof, wherein the dose amount is based on the free acid form. In some embodiments, the method comprises administering a dose amount of from about 500 mg to about 1000 mg of the compound of Formula I, or a pharmaceutically acceptable salt thereof, wherein the dose amount is based on the free acid form. In some embodiments, the method comprises administering a dose amount of from about 400 mg to about 700 mg of the compound of Formula I, or a pharmaceutically acceptable salt thereof, wherein the dose amount is based on the free acid form. In some embodiments, the method comprises administering a dose amount of from about 600 mg to about 700 mg of the compound of Formula I, or a pharmaceutically acceptable salt thereof, wherein the dose amount is based on the free acid form. In some embodiments, the method comprises administering a dose amount of from about 400 mg to about 500 mg of the compound of Formula I, or a pharmaceutically acceptable salt thereof, wherein the dose amount is based on the free acid form.

In some embodiments, the method comprises administering a dose amount of from about 5 mg to about 1000 mg of the compound of Formula I, or a pharmaceutically acceptable salt thereof, wherein the dose amount is based on the free acid form. In some embodiments, the method comprises administering a dose amount of from about 5 mg to about 750 mg of the compound of Formula I, or a pharmaceutically acceptable salt thereof, wherein the dose amount is based on the free acid form. In some embodiments, the method comprises administering a dose amount of from about 5 mg to about 650 mg of the compound of Formula I, or a pharmaceutically acceptable salt thereof, wherein the dose amount is based on the free acid form. In some embodiments, the method comprises administering a dose amount of from about 5 mg to about 500 mg of the compound of Formula I, or a pharmaceutically acceptable salt thereof, wherein the dose amount is based on the free acid form. In some embodiments, the method comprises administering a dose amount of from about 10 mg to about 450 mg of the compound of Formula I, or a pharmaceutically acceptable salt thereof, wherein the dose amount is based on the free acid form. In some embodiments, the method comprises administering a dose amount of from about 10 mg to about 250 mg of the compound of Formula I, or a pharmaceutically acceptable salt thereof, wherein the dose amount is based on the free acid form.

In some embodiments, the method comprises administering a dose amount of about 10 mg, about 40 mg, about 50 mg, about 62.5 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg of the compound of Formula I, or a pharmaceutically acceptable salt thereof, wherein the dose amount is based on the free acid form.

In some embodiments, the method comprises administering a dose amount of about 10 mg, about 30 mg, about 50 mg, about 150 mg, about 250 mg, about 325 mg, about 450 mg, about 900 mg, about 1300 mg, or about 1350 mg of the compound of Formula I, or a pharmaceutically acceptable salt thereof, wherein the dose amount is based on the free acid form. In some embodiments, the method comprises administering a dose amount of about 30 mg, about 150 mg, about 450 mg, about 900 mg, or about 1350 mg of the compound of Formula I, or a pharmaceutically acceptable salt thereof, wherein the dose amount is based on the free acid form.

In some embodiments, the method comprises administering a dose amount of about 10 mg of the compound of Formula I, or a pharmaceutically acceptable salt thereof, wherein the dose amount is based on the free acid form. In some embodiments, the method comprises administering a dose amount of about 30 mg of the compound of Formula I, or a pharmaceutically acceptable salt thereof, wherein the dose amount is based on the free acid form. In some embodiments, the method comprises administering a dose amount of about 50 mg of the compound of Formula I, or a pharmaceutically acceptable salt thereof, wherein the dose amount is based on the free acid form. In some embodiments, the method comprises administering a dose amount of about 150 mg of the compound of Formula I, or a pharmaceutically acceptable salt thereof, wherein the dose amount is based on the free acid form. In some embodiments, the method comprises administering a dose amount of about 250 mg of the compound of Formula I, or a pharmaceutically acceptable salt thereof, wherein the dose amount is based on the free acid form. In some embodiments, the method comprises administering a dose amount of about 325 mg of the compound of Formula I, or a pharmaceutically acceptable salt thereof, wherein the dose amount is based on the free acid form. In some embodiments, the method comprises administering a dose amount of about 450 mg of the compound of Formula I, or a pharmaceutically acceptable salt thereof, wherein the dose amount is based on the free acid form. In some embodiments, the method comprises administering a dose amount of about 650 mg of the compound of Formula I, or a pharmaceutically acceptable salt thereof, wherein the dose amount is based on the free acid form. In some embodiments, the method comprises administering a dose amount of about 900 mg of the compound of Formula I, or a pharmaceutically acceptable salt thereof, wherein the dose amount is based on the free acid form. In some embodiments, the method comprises administering a dose amount of about 1300 mg of the compound of Formula I, or a pharmaceutically acceptable salt thereof, wherein the dose amount is based on the free acid form. In some embodiments, the method comprises administering a dose amount of about 1350 mg of the compound of Formula I, or a pharmaceutically acceptable salt thereof, wherein the dose amount is based on the free acid form.

In some embodiments, the dose amount is administered in a single solid dosage form (e.g., one tablet). In some embodiments, the dose amount is administered in one or more solid dosage forms (e.g., two or more tablets).

In some embodiments, the pharmaceutical formulations, for example a solid dosage form (e.g., tablet) described herein comprises 10 mg of the compound of Formula I and is administered once weekly. In some embodiments, the pharmaceutical formulations, for example a solid dosage form (e.g., tablet) described herein comprises 50 mg of the compound of Formula I and is administered once weekly (QW). In some embodiments, the pharmaceutical formulations, for example a solid dosage form (e.g., tablet) described herein comprises 150 mg of the compound of Formula I and is administered once weekly (QW). In some embodiments, the pharmaceutical formulations, for example a solid dosage form (e.g., tablet) described herein comprises 250 mg of the compound of Formula I and is administered once weekly (QW). In some embodiments, the pharmaceutical formulations, for example a solid dosage form (e.g., tablet) described herein comprises 325 mg of the compound of Formula I and is administered once weekly (QW). In some embodiments, the pharmaceutical formulations, for example a solid dosage form (e.g., tablet) described herein comprises 450 mg of the compound of Formula I and is administered once weekly (QW). In some embodiments, the pharmaceutical formulations, for example a solid dosage form (e.g., tablet) described herein comprises 650 mg of the compound of Formula I and is administered once weekly (QW). In some embodiments, 650 mg of the compound of Formula I is administered once weekly with two solid dosage forms, wherein each solid dosage form (e.g., tablet) comprises 325 mg of the compound of Formula I.

In some embodiments of the methods provided herein, the human is infected with HIV. In some embodiments of the methods provided herein, the human has an HIV-1 RNA copy number of less than about 50 copies/mL (i.e., is virologically suppressed). In some embodiments of the methods provided herein, the human is infected with HIV but has not developed AIDS. In some embodiments of the methods provided herein, the human is infected with HIV and has developed AIDS. In some embodiments of the methods provided herein, the human has an HIV-1 RNA copy number of about 5000 to about 400,000 copies/mL.

In some embodiments, the human is treatment experienced, meaning that the patient has previously taken one or more forms of HIV medication. In some embodiments, the human is treatment naive. In some embodiments, the human is treatment experienced and naive to treatment with an integrase strand transfer inhibitor (INSTI). In some embodiments, the human is treatment experienced and has been off antiretroviral therapy for at least twelve weeks.

In some embodiments, the HIV infection is HIV-1 infection characterized by HIV-1 mutant resistance to one or more antiviral medications. In some embodiments, the HIV infection is an HIV-1 infection characterized by HIV-1 mutant resistance to two or more antiretroviral medications. In some embodiments, the HIV infection is an HIV-1 infection characterized by HIV-1 mutant resistance to three or more antiretroviral medications.

In some embodiments, the HIV-1 mutant is resistant to a protease inhibitor (PI), a capsid assembly inhibitor (CAI), a nucleoside or nucleotide reverse transcriptase inhibitor (NRTI), a non-nucleoside or non-nucleotide reverse transcriptase inhibitor (NNRTI), or an integrase strand transfer inhibitor (INSTI). In some embodiments, the HIV-1 mutant is resistant to a protease inhibitor (PI), a nucleoside or nucleotide reverse transcriptase inhibitor (NRTI), a non-nucleoside or non-nucleotide reverse transcriptase inhibitor (NNRTI), or an integrase strand transfer inhibitor (INSTI). In certain embodiments, the HIV-1 mutant resistant to a protease inhibitor is selected from 150V, 184V/L90M, G48V/V82A/L90M, and G48V/V82S. In certain embodiments, the HIV-1 mutant resistant to a nucleoside or nucleotide reverse transcriptase inhibitor is selected from K65R, M184V, and 6TAMs. In certain embodiments, the HIV-1 mutant resistant to a nucleoside or nucleotide reverse transcriptase inhibitor is selected from K65R, M184V, M41L/T215Y, and 6TAMs. In certain embodiments, the HIV-1 mutant resistant to a non-nucleoside or non-nucleotide reverse transcriptase inhibitor is selected from K103N, Y181C, Y188L, L100I/K103N, and K103N/Y181C. In certain embodiments, the HIV-1 mutant resistant to a capsid assembly inhibitor is selected from L561, N57H, M66I, K70N, Q67H/N74D, and Q67H/N74D.

In certain embodiments, the HIV-1 mutant resistant to an integrase strand transfer inhibitor comprises one or more integrase substitutions associated with INSTI class resistance. In certain embodiments, the HIV-1 mutant resistant to an integrase strand transfer inhibitor comprises one or two integrase substitutions associated with INSTI class resistance. In certain embodiments, the HIV-1 mutant resistant to a integrase strand transfer inhibitor is selected from Y143R, E138K/Q148K, G140S/Q148R, E92Q/N155H, N155H/Q148R, and R263K/M50I. In certain embodiments, the HIV-1 mutant resistant to a integrase strand transfer inhibitor is selected from E92Q, Y143R, Q148R, N155H, R263K, E138K/Q148K, G140S/Q148R, E92Q/N155H, N155H/Q148R, and R263K/M50I. In certain embodiments, the HIV-1 mutant resistant to an integrase strand transfer inhibitor is selected from G140A/S, Q148H/K, L741, T97A, E138A/K, S153F, S153Y, and a combination thereof. In certain embodiments, the HIV-1 mutant resistant to an integrase strand transfer inhibitor is a S153 variant.

In some embodiments, the human (or “patient”) is infected with HIV-1 resistant to at least one antiretroviral medication. In some embodiments, the patient is infected with multidrug resistant HIV-1 which is resistant to at least one antiretroviral medication from each of two different classes of antiretroviral medications. In some embodiments, the patient is infected with multidrug resistant HIV-1 which is resistant to at least one antiretroviral medication from each of three different classes of antiretroviral medications. In some embodiments, the different classes of antiretroviral medications are selected from a nucleoside or nucleotide reverse transcriptase inhibitor (NRTI), a non-nucleoside or non-nucleotide reverse transcriptase inhibitor (NNRTI), a protease inhibitor (PI), a capsid assembly inhibitor (CAI), and an integrase strand transfer inhibitor (INSTI). In some embodiments, the different classes of antiretroviral medications are selected from a nucleoside or nucleotide reverse transcriptase inhibitor (NRTI), a non-nucleoside or non-nucleotide reverse transcriptase inhibitor (NNRTI), a protease inhibitor (PI), and an integrase strand transfer inhibitor (INSTI).

In some embodiments, the NRTI is selected from emtricitabine, lamivudine (3TC), zidovudine (azidothymidine (AZT)), didanosine (ddI), dideoxyinosine, tenofovir, tenofovir alafenamide, tenofovir alafenamide hemifumarate, tenofovir disoproxil fumarate, stavudine (d4T), zalcitabine (dideoxycytidine, ddC), and abacavir.

In some embodiments, the NNRTI is selected from efavirenz, etravirine, rilpivirine, nevirapine, and delavirdine.

In some embodiments, the PI is selected from amprenavir, atazanavir, darunavir, fosamprenavir, indinavir, lopinavir, nelfinavir, ritonavir, saquinavir, and tipranavir.

In some embodiments, the CAI is lenacapavir.

In some embodiments, the INSTI is selected from raltegravir, elvitegravir, and dolutegravir.

In some embodiments of the method, the patient had been previously treated with at least one antiretroviral medication for at least 3 months, at least 6 months, at least 9 months, or at least 12 months.

In some embodiments, the patient failed a prior HIV treatment regimen including administration of at least one antiretroviral medication. In certain embodiments, the prior treatment regimen included administration of at least one antiretroviral medication from each of two different classes of antiretroviral medications. In certain embodiments, the prior treatment regimen included administration of at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI).

In some embodiments of the methods provided herein, the compound of Formula I, or a pharmaceutically acceptable salt thereof, is administered in combination with one or more additional therapeutic agents (e.g., one, two, three, or four additional therapeutic agents). In some embodiments of the methods provided herein, the method comprises administering to the human one, two, three, or four additional therapeutic agents.

In certain embodiments, the present description provides a method for treating an HIV infection, comprising administering to a patient in need thereof a therapeutically effective amount of a pharmaceutical composition (e.g., solid oral dosage form) described herein, in combination with a therapeutically effective amount of one or more additional therapeutic agents.

Combination or co-administration of the compound of Formula I, or a pharmaceutically acceptable salt thereof, with one or more additional therapeutic agents generally refers to simultaneous or sequential administration of the compound of Formula I, or a pharmaceutically acceptable salt thereof, and one or more additional therapeutic agents, such that therapeutically effective amount of the compound of Formula I, or a pharmaceutically acceptable salt thereof, and the one or more additional therapeutic agents are both present in the body of the patient. When administered sequentially, the combination may be administered in two or more administrations.

Co-administration includes administration of unit dosages of the compounds disclosed herein before or after administration of unit dosages of one or more additional therapeutic agents. For example, the compound disclosed herein may be administered within seconds, minutes, or hours of the administration of the one or more additional therapeutic agents. In some embodiments, a unit dose of a compound disclosed herein is administered first, followed within seconds or minutes by administration of a unit dose of one or more additional therapeutic agents. Alternatively, a unit dose of one or more additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of a compound disclosed herein.

In some embodiments, the additional therapeutic agent may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CDK-4 inhibitors, CDK-6 inhibitors, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, TAT protein inhibitors, Prolylendopeptidase inhibitors, Phospholipase A2 inhibitors, pharmacokinetic enhancers, HIV gene therapy, HIV vaccines, and combinations thereof.

In some embodiments, the additional therapeutic agents are selected from the group consisting of HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gpl20 inhibitors, CCR5 inhibitors, latency reversing agents, capsid polymerization inhibitors, HIV bNAbs, TLR7 agonists, pharmacokinetic enhancers, other drugs for treating HIV, or combinations thereof.

In some embodiments, the additional therapeutic agent is selected from the group consisting of combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agents are each independently selected from HIV capsid inhibitors, HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, and CCR5 inhibitors. In some embodiments, the additional therapeutic agents are each independently selected from a HIV nucleoside or nucleotide inhibitor of reverse transcriptase and an HIV capsid inhibitor or an HIV capsid polymerization inhibitor.

In some embodiments of the methods disclosed herein, the compound of Formula I or pharmaceutically acceptable salt thereof is administered in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine. In some embodiments of the methods disclosed herein, the compound of Formula I or pharmaceutically acceptable salt thereof is administered in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine.

In some embodiments of the methods disclosed herein, the compound of Formula I or pharmaceutically acceptable salt thereof is administered in combination with at least one additional therapeutic agent selected from the group consisting of:
(1) nucleoside reverse transcriptase translocation inhibitors (“NRTTIs”), such as 4′-Ethynyl-2-fluoro-2′-deoxyadenosine triphosphate (also known as MK-8591 and EFdA);
(2) nucleoside or nucleotide reverse transcriptase inhibitors (“NRTIs”), such as tenofovir alafenamide fumarate, tenofovir alafenamide, tenofovir alafenamide hemifumarate, GS-9131, and GS-9148;
(3) non-nucleoside or non-nucleotide reverse transcriptase inhibitors (“NNRTIs”), such as efavirenz, etravirine, rilpivirine, nevirapine, and delavirdine;
(4) protease Inhibitors (“PIs”), such as amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-31091; and
(5) integrase strand transfer inhibitors (“INSTIs”), such as Bictegravir, cabotegravir, raltegravir, and dolutegravir.

### HIV Combination Drug Products

Examples of combination drug products include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (KIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs

Examples of gp 120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp 160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### Rna Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18 (11): 671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL 16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the crystalline forms, salts and co-crystals as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRIKE) include, but are not limited to, OXS-3550, HIV-TriKE, and CD16-IL-15-B7H3 Trike.

Indoleamine-pyrrole-2,3-dioxygenase (IDO1) Inhibitors

In various embodiments, the crystalline forms, salts and co-crystals as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the crystalline forms, salts and co-crystals as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### Cdk Inhibitors or Antagonists

In some embodiments, the crystalline forms, salts and co-crystals described herein are combined with an inhibitor or antagonist of CDK. In some embodiments, the CDK inhibitor or antagonist is selected from the group consisting of VS2-370.

### Sting Agonists, RIG-I and NOD2 Modulators

In some embodiments, the crystalline forms, salts and co-crystals described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### Lag-3 and TIM-3 Inhibitors

In certain embodiments, the crystalline forms, salts and co-crystals as described herein are combined with an anti-TIM-3 antibody, such as TSR-022, LY-3321367, MBG-453, INCAGN-2390.

In certain embodiments, the crystalline forms, salts and co-crystals herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the crystalline forms, salts and co-crystals described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include without limitation CYT-107.

Examples of additional immune-based therapies that can be combined with the crystalline forms, salts and co-crystals of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

Phosphatidylinositol 3-kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

Alpha-4 beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al, Nucleic Acids Res., 42 (Database issue): D1 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173 (7): 1783-1795; Sajadi et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al., Nucleic Acids Res., 42 (Database issue): D1 133-9 (2014), Mascola et al., Immunol Rev., 254 (1): 225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, DID2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat, and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp 140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp 120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PTI, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, Maga Vax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp 160 HIV vaccine, RNActive HIV vaccine, SCB-703, Tat Oyi vaccine, TBC-M4, UBI HIV gp120, Vacc-4x+romidepsin, variant gp 120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb 12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (KIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the crystalline forms, salts and co-crystals disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, the crystalline forms, salts and co-crystals disclosed herein is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, the crystalline forms, salts and co-crystals disclosed herein are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the crystalline forms, salts and co-crystals described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupTI cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the crystalline forms, salts and co-crystals disclosed herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the crystalline forms, salts and co-crystals disclosed herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp 120, the CD4-induced binding site on gp 120, N glycan on gp120, the V2 of gp 120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### Tcr T-Cell Therapy

In certain embodiments, the crystalline forms, salts and co-crystals disclosed herein are combined with a population of TCR-T-cells. TCR-T-cells are engineered to target HIV derived peptides present on the surface of virus-infected cells, for example, ImmTAV.

### B-Cell Therapy

In certain embodiments, the crystalline forms, salts and co-crystals disclosed herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

The crystalline form, salt or co-crystal disclosed herein may be combined with one, two, three, or four additional therapeutic agents in any dosage amount of the crystalline form, salt or co-crystal (e.g., from 1 mg to 1000 mg of compound).

In one embodiment, kits comprising crystalline form, salt or co-crystal disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

HIV Long acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

Also provided herein are uses of any of the disclosed pharmaceutical compositions for treating or preventing an HIV infection. In some embodiments, provided herein is the use of an oral dosage form disclosed herein for treating or preventing an HIV infection.

Also provided herein are any of the pharmaceutical compositions provided herein for use in a method for treating or preventing HIV infection. In some embodiments, provided herein are any of the solid oral dosage forms disclosed herein for use in a method for treating or preventing HIV infection.

Also provided herein are uses of any of the pharmaceutical compositions provided herein in the manufacture of a medicament for treating or preventing an HIV infection in a human. In some embodiments, provided herein are uses of any of the solid oral dosage forms provided herein in the manufacture of a medicament for treating or preventing an HIV infection in a human.

### V. Methods of Manufacture

Methods for producing the pharmaceutical formulations, for example the solid oral dosage forms (e.g., tablets) disclosed herein are also provided.

In general, tableting methods are well known in the art of pharmacy. Techniques and formulations generally are found in Remington's Pharmaceutical Sciences (Mack Publishing Co., Easton, PA), which is hereby incorporated by reference herein in its entirety.

A tablet can be made by compression or molding, optionally with one or more excipients. Compressed tablets may be prepared by compressing in a suitable machine the active ingredient in a free-flowing form such as a powder or granules, optionally mixed with excipients.

In some embodiments, the pharmaceutical composition, solid oral dosage form, or tablet is manufactured using a method comprising dry granulation. The dry granulation may be carried out by roller compaction. The dry granulation may be performed on a blend of the compound of Formula I, the filler, the disintegrant, the lubricant, and the binder. In some embodiments, the composition comprises microcrystalline cellulose and lactose as a filler, croscarmellose sodium as a disintegrant, magnesium stearate as a lubricant, and hydroxypropyl cellulose as a binder.

In some embodiments, the pharmaceutical composition, solid oral dosage form, or tablet is manufacturing using a method comprising wet granulation.

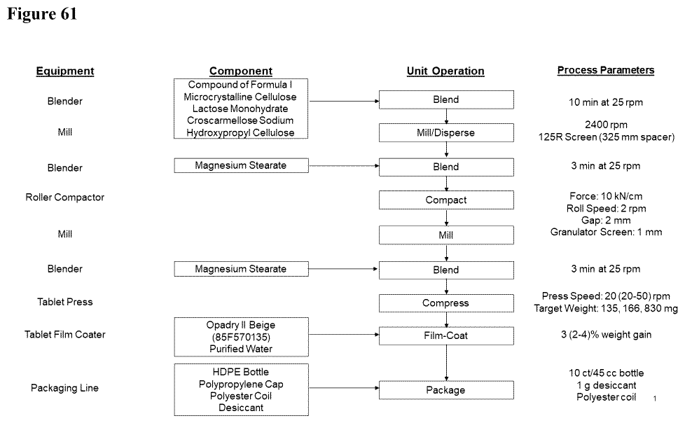

In some embodiments, the method comprises mixing the drug substance with the excipients. In some embodiments the manufacturing process comprises co-blending and lubricating the compound of Formula I with intragranular excipients. In some embodiments, the method further comprises roller compaction and/or milling. The resulting Formula I granules are then blended with extragranular excipients. The resulting mixture is then compressed into core tablets. In some embodiments, the tablets are further coated with a film coat. An exemplary manufacturing process is shown in FIG. 61.

### EXAMPLES

### General Methods

XRPD patterns were collected with a PANalytical Empyrean diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcel 1D-Medipix3 PASS (programmable anti-scatter slit), located 240 mm from the sample and Data Collector software v. 7.2b.

DSC was run on a Q2000 (TA Instruments, New Castle, DE) by loading 1-10 mg of material into a crimped or open Tzero standard aluminum pan and heating the sample at 10° C./min from 20 to 300° C. or above. The sample and reference pans were under a 50 mL/min nitrogen purge. Data analysis was completed using Universal Analysis 2000 Version 4.5A (TA Instruments, New Castle, DE).

TGA was used to evaluate sample weight loss as a function of temperature on either a Q5000 or Q500 (TA Instruments, New Castle, DE), by loading 1-10 mg of material onto a weigh pan and heating the sample to 350° C. or above at a rate of 10° C./min. The sample and reference pans were under a 60 mL/min and 40 mL/min nitrogen purge, respectively. Data analysis was completed using Universal Analysis 2000 Version 4.5A (TA Instruments, New Castle, DE).

Hygroscopicity was studied using dynamic vapor sorption (DVS, TA Q5000 SA, TA Instruments, New Castle, DE or DVS, DVS Intrinsic, Surface Measurement Systems, London, UK). A sample (1-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5% or starting at 10% RH and increasing to 90% RH and then back down to 10% RH. Each relative humidity increment had an equilibration time of 180 minutes, unless weight change % was less than 0.002% in 30 minutes. Data analysis was performed using Universal Analysis 2000 Version 4.7A (TA Instruments, New Castle, DE) for TA DVS runs and Microsoft Excel for SMS DVS runs.

### Example 1. Compound of Formula I, Form I

Compound A was dissolved in NMP with LiCl. Reaction was heated to 100 C for about 3 hours. The reaction was cooled to room temperature and about 80 mL of 0.5M HCl was added dropwise. About 20 mL water was added and the reaction was stirred for about 1 hour. EtOAc was used to extract the product, followed by the addition of NaCl to saturate the aq. layer and another extraction with EtOAc. The EtOAc layers were combined with brine, dried over sodium sulfate, filtered, and concentrated under vacuum. Silica gel column chromatography was used to purify the product. Fractions were combined and recrystallized from EtOAc/Hexanes. The product was dissolved in EtOAc and decoloring carbon was added. This was stirred for 30 minutes, then filtered and washed with EtOAc and MeOH. The solution was concentrated under vacuum and solid material was obtained of Formula I.

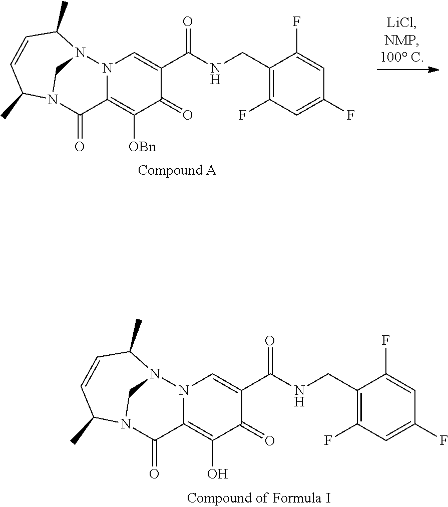

A solution of Compound B in ethyl acetate was charged to a reactor. N-methyl-2-pyrrolidinone (3 V) was charged to the solution. The solution was then distilled to about 4V. LiCl (3 equiv.) was then charged before adjusting the temperature to about 80° C. for about 4 hours. The reaction was then cooled to about 20° C. before washing the organic layer with HCl (0.5M, 5 V), NaCl (5%, 10 V), and water (5 V). The organic layer was then distilled to about 6 V before charging 2-propanol (10 V). The solution was then distilled to about 6 V before charging more 2-propanol (2 V). The temperature was then adjusted to about 60° C. for about 1 hour before adjusting to about 20° C. over about 2 hours. The slurry was aged for about 1 hour to overnight before putting the slurry through a high shear wet mill for about 1 hour. The slurry was then filtered and the cake was rinsed with isopropanol before drying at about 40° C.

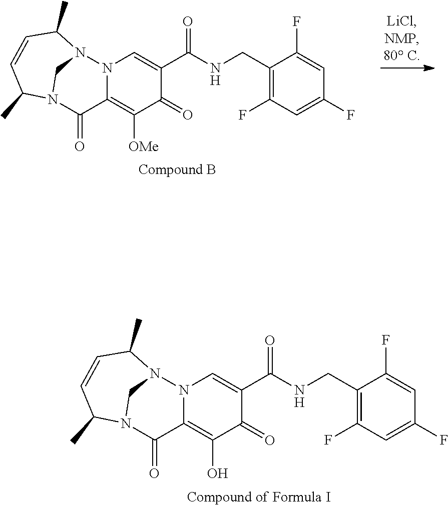

The Compound of Formula I, Form I XRPD pattern is shown in FIG. 1, and is characterized by Tier 1 reflections at 7.0, 27.9, 13.9° 2θ, but also Tier 2 at 12.3, 24.6, 17.4° 2θ, and Tier 3 29.2, 23.2, 21.4° 2θ. A list of 2-theta peaks is provided below:

Peak Table

| Pos. | Rel. Int. |
|---|---|
| [°2Th.] | [%] |
| 7.0 | 100 |
| 12.3 | 44 |
| 13.9 | 57 |
| 15.9 | 9 |
| 16.7 | 10 |
| 17.1 | 34 |
| 17.4 | 40 |
| 20.1 | 8 |
| 21.4 | 12 |
| 22.6 | 4 |
| 23.2 | 18 |
| 24.6 | 41 |
| 25.6 | 7 |
| 26.3 | 5 |
| 26.7 | 7 |
| 27.8 | 42 |
| 27.9 | 76 |
| 29.2 | 20 |
| 31.2 | 4 |
| 35.0 | 12 |
| 36.6 | 2 |
| 37.4 | 2 |

The DSC curve is shown in FIG. 2 and displays one endothermic transition at about 192° C. The TGA curve is shown in FIG. 3 and indicates that the phase is unsolvated. The DVS curve is shown in FIG. 4 and the data indicate that the form absorbs about 0.15% of water up to 95% RH at 25° C. The material was found to not have changed form post experiment.

Single crystal data was collected on the compound of Formula I, Form I and the data are summarized Table 1 and FIG. 5.

The crystal system is monoclinic and the space group is P21. The cell parameters and calculated volume are: a=8.9146 (2) Å, b=8.6717 (2) Å, c=12.7101 (3) Å, α=90°, β=93.3170 (10)°, γ=90°, V=980.95 (4) Å³. The molecular weight is 448.40 g mol⁻¹ with Z=2, resulting in a calculated density of 1.518 g cm⁻³.

### Example 2. Compound of Formula I, Form II

Compound of Formula I, Form II was isolated when excess Compound of Formula I, Form I was added to about 0.5 mL THF to create a slurry, then the slurry was stirred at room temperature for about 6 days. The slurry was filtered and XRPD was taken of the wet solids. The sample was dried in a vacuum oven then further characterized.

The XRPD pattern for the compound of Formula I, Form II is shown in FIG. 6 and is characterized by Tier 1 reflections at 5.7, 19.9, 26.8° 2θ, but also Tier 2 at 28.6, 11.4, 17.6° 2θ, and Tier 3 at 22.3, 25.1, 10.7° 2θ. A list of 2-theta peaks is provided below:

Peak Table

| Pos. | Rel. Int. |
|---|---|
| [°2Th.] | [%] |
| 5.7 | 100 |
| 10.7 | 11 |
| 11.4 | 23 |
| 11.7 | 11 |
| 13.3 | 8 |
| 14.9 | 4 |
| 16.5 | 3 |
| 17.1 | 7 |
| 17.6 | 19 |
| 19.6 | 9 |
| 19.9 | 35 |
| 21.4 | 4 |
| 22.3 | 14 |
| 22.9 | 3 |
| 23.6 | 3 |
| 24.5 | 7 |
| 25.1 | 12 |
| 25.5 | 5 |
| 26.8 | 25 |
| 28.6 | 25 |
| 30.5 | 4 |
| 30.9 | 4 |
| 34.7 | 2 |

The DSC curve for the compound of Formula I, Form II is shown in FIG. 7 and displays an endothermic transition at about 194° C. The TGA curve for the compound of Formula I, Form II is shown in FIG. 8 and indicates that the phase is unsolvated.

### Example 3. Compound of Formula I, Sodium Salt, Form I

Compound of Formula I, sodium salt, Form I was isolated when about 1000 mg Compound of Formula I, Form I was dissolved in about 6 mL methanol at about 45° C. About 133 mg of sodium hydroxide was added. The sample was sonicated for about 1 hour, then left to stir at room temperature as an open vial until all the sample was evaporated and dry solids remained. The dry solids were combined with about 1 mL methanol and about 3 mL water and sonicated for about 30 minutes, then left to stir at room temperature as an open vial until all the sample was evaporated and dry solids remained. The dry solids were combined with about 1 mL methanol and about 3 mL water and sonicated for about 30 minutes, then left to stir at room temperature as an open vial until all the sample was evaporated and dry solids remained. The dry solids were reslurried in 30% methanol in water at room temperature for about 3 weeks. The slurry was filtered and dried in a vacuum oven overnight at about 50° C.

The Compound of Formula I, sodium salt, Form I XRPD pattern is shown in FIG. 9 and is characterized by Tier 1 reflections at 13.1, 26.8, 14.8° 2θ, but also Tier 2 at 28.2, 20.7, 17.8° 2θ, and Tier 3 at 6.2, 21.2, 23.3° 2θ. A list of 2-theta peaks is provided below:

Peak Table

| Pos. | Rel. Int. |
|---|---|
| [°2Th.] | [%] |
| 4.9 | 11 |
| 6.2 | 47 |
| 7.7 | 21 |
| 9.9 | 22 |
| 11.9 | 22 |
| 12.4 | 34 |
| 13.1 | 100 |
| 14.8 | 76 |
| 16.6 | 32 |
| 17.8 | 50 |
| 18.7 | 20 |
| 19.7 | 27 |
| 20.7 | 54 |
| 21.2 | 43 |
| 21.9 | 40 |
| 23.3 | 35 |
| 25.2 | 32 |
| 25.6 | 28 |
| 26.8 | 79 |
| 28.2 | 73 |
| 29.9 | 23 |
| 36.6 | 15 |

The DSC curve is shown in FIG. 10 and displays an endothermic transition at about 25° C., 81° C., and 124° C., and exothermic transition at about 151° C. The TGA curve is shown in FIG. 11 and indicates that there is weight loss starting at around ambient temperature. The DVS curve is shown in FIG. 12 and the data indicate that the form absorbs about 4% of water up to 95% RH at 25° C. The material was found to not have changed form post experiment.

### Example 4. Compound of Formula I, Sodium Salt, Form II

Compound of Formula I, sodium salt, Form II was isolated when about 1000 mg Compound of Formula I, Form I was added to a vial with about 19 mL of methanol. In a separate vial, about 1 mL water was combined with 2 mol equiv. sodium hydroxide to create a solution. The two solutions were combined, and 1 mL was withdrawn and dispensed into a 4 mL vial. A miVac system was used to remove the solvent resulting in a dry solid in the vial. About 1 mL water was added to the dry solids and stirred at room temperature overnight. The resulting solids were suction filtered and XRPD was taken of the wet solids. The solids were then dried and further characterized.

Compound of Formula I, sodium salt, Form II XRPD pattern is shown in FIG. 13 and is characterized by reflections at 5.3°, 7.1°, 13.4°, and 10.7° 2θ. A list of 2-theta peaks is provided below.

Peak Table

| Pos. | Rel. Int. |
|---|---|
| [°2Th.] | [%] |
| 5.3 | 100 |
| 7.1 | 44 |
| 10.7 | 15 |
| 13.4 | 41 |

The DSC curve for the Compound of Formula I, sodium salt, Form II is shown in FIG. 14 and displays an endothermic transition at about 32° C. and an endothermic transition at about 122° C. The TGA curve for the Compound of Formula I, sodium salt, Form II is shown in FIG. 15 and indicates that the phase loses mass starting at about ambient temperature.

### Example 5. Compound of Formula I, Sodium Salt, Form III

Compound of Formula I, sodium salt, Form III was isolated when about 0.1 g Compound of Formula I, Form I and about 2.5 equiv. of sodium acetate was combined with 4 volumes of isopropyl acetate and stirred with a stir bar. A solution was observed. Heptane was added dropwise until solids precipitated out of solution. The solids were filtered and characterized by XRPD.

Compound of Formula I, sodium salt, Form III XRPD pattern is shown in FIG. 16 and is characterized by Tier 1 reflections at 7.9, 8.8, 23.1° 2θ, and 26.2, 26.6, 30.3 Tier 2 at ° 20. A list of 2-theta peaks is provided below.

Peak Table

| Pos. | Rel. Int. |
|---|---|
| [°2Th.] | [%] |
| 7.9 | 18 |
| 8.8 | 100 |
| 23.1 | 17 |
| 26.2 | 9 |
| 26.6 | 7 |
| 30.3 | 5 |

### Example 6. Compound of Formula I, Sodium Salt, Form IV

Compound of Formula I, sodium salt, Form IV was combined with about 1.1 equiv sodium hydroxide, 4.35 V water, and 2.3 V methanol in a vial with a stir bar. The vial was placed on a hot plate at about 50° C. Seeds of Compound of Formula I sodium were charged and a slurry was observed. The vial was removed from heat. Solids were filtered and the filter cake was rinsed with water. Solids were dried in a vacuum oven at about 50° C. several days. XRPD data was collected.

Compound of Formula I, sodium salt, Form IV XRPD pattern is shown in FIG. 17 and is characterized by Tier 1 reflections at 5.3, 6.4, 12.8° 2θ, and Tier 2 at 14.1, 24.5, 26.4° 2θ, and Tier 3 at 8.4, 15.8, 20.7° 2θ. A list of 2-theta peaks is provided below.

Peak Table

| Pos. | Rel. Int. |
|---|---|
| [°2Th.] | [%] |
| 5.3 | 100 |
| 6.4 | 41 |
| 8.4 | 13 |
| 10.5 | 11 |
| 12.8 | 30 |
| 14.1 | 24 |
| 15.8 | 18 |
| 17.5 | 11 |
| 18.6 | 8 |
| 19.4 | 6 |
| 20.7 | 21 |
| 21.7 | 17 |
| 22.8 | 17 |
| 23.2 | 17 |
| 24.5 | 23 |
| 26.4 | 27 |
| 27.5 | 8 |
| 29.7 | 7 |

### Example 7. Compound of Formula I, Potassium Salt, Form I

Compound of Formula I, potassium salt, Form I was isolated when approximately 1000 mg of compound of Formula I, Form I was dissolved in about 6 mL acetone at about 45° C. About 188 mg of potassium hydroxide was added and the sample was sonicated for about 1 hour then left to stir at room temperature. The slurry was filtered and dried in a vacuum oven overnight at about 50° C.

The XRPD pattern for the Compound of Formula I, potassium salt, Form I is shown in FIG. 18 and is characterized by Tier 1 reflections at 7.1°, 20.1°, 25.8° 2θ, but also Tier 2 at 6.2°, 19.2°, 13.0° 2θ, and Tier 3 at 15.5°, 22.9°, 14.2° 2θ. A list of 2-theta peaks is provided below.

Peak Table

| Pos. | Rel. Int. |
|---|---|
| [°2Th.] | [%] |
| 6.2 | 85 |
| 7.1 | 100 |
| 13.0 | 60 |
| 14.2 | 47 |
| 15.5 | 57 |
| 16.4 | 45 |
| 18.1 | 45 |
| 19.2 | 63 |
| 20.1 | 98 |
| 21.9 | 45 |
| 22.9 | 55 |
| 23.7 | 28 |
| 25.8 | 90 |
| 28.6 | 21 |
| 32.1 | 29 |
| 33.8 | 25 |

The DSC curve for compound of Formula I, potassium salt Form I, is shown in FIG. 19 and displays an endothermic transition at about 17° C. and an endothermic transition at about 230° C. The TGA curve for compound of Formula I, potassium salt Form I, is shown in FIG. 20 and indicates that the phase loses mass in multiple stages with one stage starting at about ambient temperature and the next stage starting at about 100° C. The DVS curve for compound of Formula I, potassium salt Form I, is shown in FIG. 21 and the data indicate that the form absorbs more than about 18% of water up to 95% RH at 25° C. The material was found to not have changed form post experiment.

The Single crystal data was collected on compound of Formula I, potassium salt Form I, and the data are summarized in Table 2 and FIG. 22. The crystal system is trigonal and the space group is P3221. The cell parameters and calculated volume are: a=28.6496 (4) Å, b=28.6496 (4) Å, c=6.89340 (10) Å, α=90°, β=90°, γ=120°, V=4900.06 (15) Å³. The molecular weight is 494.49 g mol⁻¹ with Z=6, resulting in a calculated density of 1.005 g cm⁻³.

### Example 8. Compound of Formula I, Potassium Salt, Form II

Compound of Formula I, potassium salt, Form II was isolated when Compound of Formula I, Form I was placed in a vial with EtOH and a magnetic stir bar. Some potassium acetate was added and the experiment was stirred at room temperature. The solids were characterized by XRPD and TGA.

The Compound of Formula I, potassium salt Form II XRPD pattern is shown in FIG. 23 and is characterized by Tier 1 reflections at 30.7°, 6.8°, and 31.4° 2θ, but also Tier 2 at 12.5°, 32.6°, and 28.0° 2θ, and Tier 3 at 24.2°, 14.3°, and 25.7° 2θ.

The TGA curve is shown in FIG. 24 and indicates that the phase loses mass in multiple stages with one stage starting at about ambient temperature and the next stage starting at about 100° C. A list of 2-theta peaks is provided below.

Peak Table

| Pos. | Rel. Int. |
|---|---|
| [°2Th.] | [%] |
| 6.8 | 94 |
| 12.5 | 41 |
| 14.3 | 11 |
| 20.3 | 8 |
| 24.2 | 14 |
| 25.7 | 10 |
| 26.9 | 6 |
| 28.0 | 21 |
| 29.7 | 10 |
| 30.7 | 100 |
| 31.4 | 43 |
| 32.3 | 12 |
| 32.6 | 23 |
| 35.0 | 9 |

### Example 9. Compound of Formula I, Potassium Salt, Form III

Compound of Formula I, potassium salt, Form III was isolated when 8.5 g of Compound of Formula I, Form I and 2.4 mL (about 1 equiv) potassium hydroxide in water (50 m/v %) was combined with 5 V water in a bottle with a stir bar. A thin slurry was observed. After overnight stirring the slurry was thicker and an additional volume of water was added. Slurry was immobile. Slurry was sonicated for 1 hour, seeded, then continued to stir. This was repeated daily for about 8 days. About 1 mL of slurry was combined with 0.1 mL methanol, then heated to 50° C. A solution formed. The solution was removed from the heat. Seed was added. About 0.3 mL methanol was added. Solids were observed and filtered. Solids characterized by XRPD.

The Compound of Formula I, potassium salt Form III XRPD pattern is shown in FIG. 25 and is characterized by Tier 1 reflections at 5.2, 13.2, 13.7° 2θ, but also Tier 2 at 11.2, 12.4, 18.7° 2θ, and Tier 3 at 6.5 and 15.0° 2θ. A list of 2-theta peaks is provided below:

Peak Table

| Pos. | Rel. Int. |
|---|---|
| [°2Th.] | [%] |
| 5.2 | 100 |
| 6.5 | 13 |
| 11.2 | 27 |
| 12.4 | 25 |
| 13.2 | 31 |
| 13.7 | 58 |
| 15.0 | 14 |
| 18.7 | 22 |

### Example 10. Compound of Formula I, Diethylamine salt

Compound of Formula I, diethylamine salt was isolated when about 1000 mg of Compound of Formula I, Form I was dissolved in about 6 mL of acetone at about 45° C. About 0.25 mL of diethylamine was added and the sample was sonicated for about 15 min to yield a slurry. The sample was left to stir at room temperature. The slurry was filtered and dried in a vacuum oven overnight at about 50° C.

The XRPD pattern for the compound of Formula I, diethylamine salt is shown in FIG. 26 and is characterized by Tier 1 reflections at 19.5°, 9.7°, and 20.5° 2θ, but also Tier 2 at 21.9°, 26.5°, and 27.2° 2θ, and Tier 3 at 17.1°, 6.2°, and 10.7° 2θ. A list of 2-theta peaks is provided below:

Peak Table

| Pos. | Rel. Int. |
|---|---|
| [°2Th.] | [%] |
| 6.2 | 29 |
| 9.0 | 24 |
| 9.7 | 72 |
| 10.7 | 28 |
| 11.9 | 20 |
| 12.4 | 9 |
| 13.2 | 13 |
| 15.7 | 19 |
| 17.1 | 32 |
| 17.8 | 25 |
| 18.6 | 26 |
| 19.5 | 100 |
| 20.5 | 69 |
| 21.5 | 49 |
| 21.9 | 68 |
| 23.2 | 15 |
| 24.7 | 21 |
| 26.5 | 48 |
| 27.2 | 38 |
| 30.7 | 23 |
| 32.5 | 13 |
| 35.4 | 7 |

The DSC curve for the compound of Formula I, diethylamine salt is shown in FIG. 27 and displays an endothermic transition at about 140° C. The TGA curve for the compound of Formula I, diethylamine salt is shown in FIG. 28 and indicates that the phase loses mass in multiple stages with one stage starting at about 100° C. and the next stage starting at about 175° C. The DVS curve for the compound of Formula I, diethylamine salt is shown in FIG. 29 and the data indicate that the form absorbs less than about 1% of water up to 95% RH at 25° C. The material was found to not have changed form post experiment.

### Example 11. Compound of Formula I, Ammonia Salt

Compound of Formula I, ammonia salt was isolated when about 1000 mg of the compound of Formula I, Form I was dissolved in about 6 mL acetone at 45° C. About 100 uL ammonium hydroxide was added. The sample was sonicated for about 15 minutes and then left to stir at room temperature. The slurry was filtered and dried in a vacuum oven overnight at about 50° C.

The ammonia salt XRPD pattern is shown in FIG. 30 and is characterized by Tier 1 reflections at 12.1, 6.9, 12.5° 2θ, but also Tier 2 at 18.4, 21.0, 25.4° 2θ, and Tier 3 at 9.2, 23.1, 30.6° 2θ. A list of 2-theta peaks is provided below:

Peak Table

| Pos. | Rel. Int. |
|---|---|
| [°2Th.] | [%] |
| 6.9 | 56 |
| 9.2 | 6 |
| 12.1 | 100 |
| 12.5 | 38 |
| 15.6 | 2 |
| 17.1 | 4 |
| 18.4 | 25 |
| 21.0 | 15 |
| 23.1 | 6 |
| 25.4 | 11 |
| 26.4 | 5 |
| 30.6 | 6 |

The DSC curve is shown in FIG. 31 and displays an endothermic transition at about 121° C. and an endothermic transition at about 194° C. The TGA curve is shown in FIG. 32 and indicates that the phase loses mass starting at about 80° C. The DVS curve is shown in FIG. 33 and the data indicate that the form absorbs less than about 1% of water up to 95% RH at 25° C. The material was found to not have changed form post experiment.

### Example 12. Compound of Formula I, Calcium Salt, Form I

Compound of Formula I, calcium salt, Form I was isolated when about 1000 mg of compound of Formula I, Form I was dissolved in about 6 mL acetone at 45° C. Separately, about 200 mg of potassium hydroxide was dissolved in about 6 mL water. The two solutions were combined and then about 124 mg of calcium chloride was added to yield a precipitate. About 28 mL of water was added and the sample was sonicated for about 30 minutes then left to stir at room temperature. The slurry was filtered and dried in a vacuum oven overnight at about 50° C.

The calcium salt, Form I XRPD pattern can be found in FIG. 34. The DSC curve is shown in FIG. 35 and displays a glass transition at about 130° C.

### Example 13. Compound of Formula I, Calcium Salt, Form II

Compound of Formula I, calcium salt, Form II was isolated when about 0.02 g of Compound B:

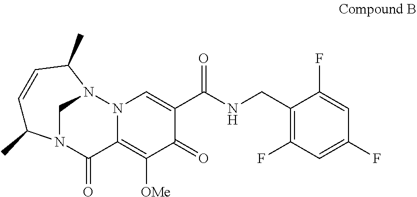

and about 0.02 g of calcium chloride was added to a vial with a stir bar and 10 V isopropanol. The vial was placed on a hot plate at 80° C. A solution was observed. Vial was removed from heat. Solids were observed and were filtered. XRPD was taken of the solids.

The calcium salt, Form II XRPD pattern is shown in FIG. 36 and is characterized by Tier 1 reflections at 7.1, 8.5, 11.7° 2θ, and Tier 2 at 14.1, 16.5, 20.1° 2θ. A list of 2-theta peaks is provided below:

Peak Table

| Pos. | Rel. Int. |
|---|---|
| [°2Th.] | [%] |
| 7.1 | 100 |
| 8.5 | 7 |
| 11.7 | 5 |
| 14.1 | 6 |
| 16.5 | 7 |
| 20.1 | 7 |
| 21.5 | 5 |
| 24.8 | 5 |

### Example 14. Compound of Formula I, Calcium Salt, Form III

Compound of Formula I, calcium salt, Form III was isolated when about 1 g of Compound B:

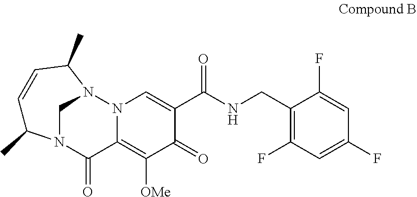

and about 0.7 g of calcium bromide was added to a vial with a stir bar and 4 V isopropanol. The vial was placed on a hot plate at 80° C. A solution was observed. After stirring for about an hour solids were observed. Vial was removed from heat. Solids were observed and were filtered. XRPD was taken of the solids.

The calcium salt, Form III XRPD pattern is shown in FIG. 37 and is characterized by Tier 1 reflections at 6.3, 7.2, 25.5° 2θ, and Tier 2 at 21.2, 28.4, 31.4° 2θ. A list of 2-theta peaks is provided below:

Peak Table

| Pos. | Rel. Int. |
|---|---|
| [°2Th.] | [%] |
| 6.3 | 100 |
| 7.2 | 35 |
| 21.2 | 11 |
| 25.5 | 22 |
| 28.4 | 11 |
| 31.4 | 9 |

### Example 15. Compound of Formula I, Magnesium Salt, Form I

Compound of Formula I, magnesium salt, Form I was isolated when about 1000 mg of compound of Formula I, Form I was dissolved in about 6 mL acetone at 45° C. Separately, about 200 mg of potassium hydroxide was dissolved in about 6 mL water. The two solutions were combined and then about 0.55 mL of 2 M magnesium chloride was added to yield a precipitate. About 28 mL of water was added and the sample was sonicated for about 30 minutes then left to stir at room temperature. The slurry was filtered and dried in a vacuum oven overnight at about 50° C.

The XRPD pattern can be found in FIG. 38. The DSC curve is shown in FIG. 39 and displays a glass transition at about 155° C.

### Example 16. Compound of Formula I, Magnesium Salt, Form II

Compound of Formula I, magnesium salt, Form II was isolated when about 1 g of compound of Compound B:

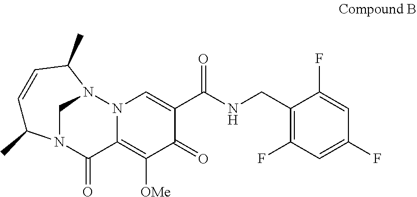

and about 1.3 g of magnesium bromide hexahydrate was added to a vial with a stir bar and 3 V isopropanol. The vial was placed on a hot plate at 90° C. Mostly a solution was observed. Solids were observed. Vial was removed from heat. Solids were observed and were filtered. XRPD was taken of the solids.

The XRPD pattern is shown in FIG. 40 is characterized by Tier 1 reflections at 7.0, 19.8, 24.3° 2θ, but also Tier 2 at 6.5, 13.0, 21.2° 2θ. A list of 2-theta peaks is provided below:

Peak Table

| Pos. | Rel. Int. |
|---|---|
| [°2Th.] | [%] |
| 6.5 | 51 |
| 7.0 | 88 |
| 13.0 | 65 |
| 19.8 | 100 |
| 21.2 | 44 |
| 24.3 | 77 |
| 25.9 | 38 |

### Example 17. Compound of Formula I, N-Butylamine Salt

Compound of Formula I, N-butylamine salt was isolated when compound of Formula I, Form I was placed in a vial with EtOH and a magnetic stir bar. Some N-butylamine was added and the experiment was stirred at room temperature. The solids were characterized by XRPD. The solids were dried then further characterized.

The XRPD pattern is shown in FIG. 41 and is characterized by Tier 1 reflections at 8.0, 10.7, 5.8° 2θ, but also Tier 2 at 19.2, 9.7, 11.6° 2θ, and Tier 3 at 18.7, 26.5, 22.3° 2θ. A list of 2-theta peaks is provided below:

Peak Table

| Pos. [°2Th.] | Rel. Int. [%] |
|---|---|
| 5.8 | 48 |
| 8.0 | 100 |
| 9.7 | 45 |
| 10.2 | 17 |
| 10.7 | 78 |
| 11.6 | 26 |
| 16.0 | 5 |
| 16.7 | 10 |
| 18.7 | 16 |
| 19.2 | 45 |
| 20.3 | 11 |
| 22.3 | 13 |
| 23.0 | 3 |
| 23.7 | 11 |
| 25.3 | 10 |
| 26.1 | 10 |
| 26.5 | 14 |
| 27.9 | 7 |
| 31.6 | 6 |

The DSC curve is shown in FIG. 42 and displays an endothermic transition at about 166° C. The TGA curve is shown in FIG. 43 and indicates that the phase loses mass in multiples stages starting at about 80° C. and another stage starting at about 175° C. The DVS curve is shown in FIG. 44 and the data indicate that the form absorbs less than about 0.2% of water up to 95% RH at 25° C. The material was found to not have changed form post experiment.

### Example 18. Compound of Formula I, Diethanolamine Salt

Compound of Formula I, diethanolamine salt was isolated when about 17.4 g of compound of Formula I, Form I was added to a 100 ml bottle with a large stir bar and about 60 mL of isopropanol and about 4.1 g diethanolamine. A solution with some solids was observed. Seed was added. After about an hour of stirring, 15 mL heptane was added along with an additional seed charge. Slurry was stirred for about 24 hours. Solids were suction filtered and the cake was rinsed with isopropanol. The solids were placed in a vacuum oven at about 40° C. Solids were characterized by XRPD.

The XRPD pattern is shown in FIG. 45 and is characterized by Tier 1 reflections at 6.1, 18.4, 19.5° 2θ, but also Tier 2 at 9.9, 20.4, 21.8° 2θ, and Tier 3 at 16.9, 18.9, 26.5° 2θ. A list of 2-theta peaks is provided below:

Peak Table

| Pos. [°2Th.] | Rel. Int. [%] |
|---|---|
| 6.1 | 100 |
| 9.9 | 16 |
| 11.9 | 9 |
| 13.2 | 1 |
| 15.6 | 12 |
| 16.9 | 13 |
| 18.4 | 79 |
| 18.9 | 14 |
| 19.5 | 38 |
| 20.4 | 20 |
| 21.4 | 19 |
| 21.8 | 29 |
| 22.9 | 5 |
| 24.6 | 11 |
| 26.5 | 15 |
| 30.5 | 10 |
| 33.5 | 2 |

### Example 19. Compound of Formula I, Ethylenediamine Salt

Compound of Formula I, ethylenediamine salt was isolated when about 1.2 g of compound of Formula I, Form I was combined with about 66 mL methanol in a 100 ml bottle with stir bar. About 0.08 g of ethylenediamine was combined with about 3.3 mL methanol. The ethylenediamine solution was charged to the compound of Formula I, Form I solution. Immediate precipitation was observed. The solids were suction filtered and placed in a vacuum oven for about 18 hours at about 40° C. XRPD was taken of the solids.

The XRPD pattern is shown in FIG. 46 and is characterized by Tier 1 reflections at 3.5, 6.9, 12.0° 2θ, but also Tier 2 at 9.2, 12.5, 20.2° 2θ. A list of 2-theta peaks is provided below:

Peak Table

| Pos. [°2Th.] | Rel. Int. [%] |
|---|---|
| 3.5 | 100 |
| 6.9 | 6 |
| 9.2 | 1 |
| 12.0 | 4 |
| 12.5 | 1 |
| 15.2 | 1 |
| 19.4 | 1 |
| 20.2 | 1 |

### Example 20. Compound of Formula I, Morpholine Salt

Compound of Formula I, morpholine salt was isolated when about 15 g of compound of Formula I, Form I was combined in a 100 ml bottle with a large stir bar with about 93 mL acetonitrile and about 4.5 g of 2-morpholinoethanol. Mostly a solution with some solids was observed. Seeds were added. Slurry stirred for about 24 hours. The slurry was suction filtered and the cake was rinsed with acetonitrile. The filtered solids were placed in a vacuum oven at 40° C. for about 24 hours. XRPD was taken of the solids.

The XRPD pattern is shown in FIG. 47 and is characterized by Tier 1 reflections at 14.3, 19.0, 22.7° 2θ, but also Tier 2 at 9.0, 14.5, 22.3° 2θ, and Tier 3 at 4.7, 17.2, 26.0° 2θ. A list of 2-theta peaks is provided below:

Peak Table

| Pos. [°2Th.] | Rel. Int. [%] |
|---|---|
| 4.7 | 15 |
| 9.0 | 47 |
| 9.3 | 32 |
| 11.0 | 15 |
| 13.9 | 18 |
| 14.3 | 100 |
| 14.5 | 69 |
| 17.2 | 40 |
| 18.5 | 28 |
| 19.0 | 49 |
| 19.3 | 28 |
| 22.3 | 47 |
| 22.7 | 50 |
| 26.0 | 31 |
| 28.4 | 24 |
| 29.2 | 19 |
| 29.9 | 28 |
| 36.2 | 11 |

### Example 21. Compound of Formula I, trans-ferulic Co-crystal, Form I

Compound of Formula I, trans-ferulic co-crystal, Form I was first isolated when about 50 mg of compound of Formula I was combined in a vial with about 1 mL methyl tetrahydrofuran or about 1 mL ethyl acetate and trans-ferulic acid. The cap was removed from the vial and the solvent was allowed to evaporate at room temperature. After the solvent was removed, dry solids remained. The solids were characterized by XRPD.

Compound of Formula I, trans-ferulic co-crystal, Form I was also isolated when about 8.5 g of compound of Formula I, Form I, about 4.2 g of ferulic acid, and about 250 mL isopropyl acetate was charged to a 250 mL reactor with the jacket temperature set to about 70° C. A solution was achieved at an internal temperature of about 71° C. The jacket temperature was lowered to about 45° C. and seeds were added. Excess ferulic acid was charged and the system was heat cycled up about 70° C. and down to about 45° C. twice. The system was then cooled to about 20° C. over about 3 hours. The solids were filtered and the cake was rinsed with isopropyl acetate. The solids were dried in a vacuum oven at about 30° C.

The Compound of Formula I, trans-ferulic co-crystal, Form I XRPD pattern is shown in FIG. 51 and is characterized by Tier 1 reflections at 6.4, 25.0, 16.3° 2θ, but also Tier 2 at 24.2, 28.8, 8.7° 2θ, and Tier 3 at 21.3, 26.9, 22.7° 2θ. A list of 2-theta peaks is provided below:

Peak Table

| Pos. [°2Th.] | Rel. Int. [%] |
|---|---|
| 6.4 | 100 |
| 7.6 | 9 |
| 8.7 | 23 |
| 10.7 | 14 |
| 13.7 | 9 |
| 14.2 | 11 |
| 15.1 | 14 |
| 16.3 | 38 |
| 18.9 | 10 |
| 19.5 | 6 |
| 21.3 | 20 |
| 22.7 | 15 |
| 24.2 | 31 |
| 25.0 | 74 |
| 26.9 | 19 |
| 28.0 | 7 |
| 28.8 | 28 |

The DSC curve is shown in FIG. 52 and displays an endothermic transition at about 139° C. and an endothermic transition at about 180° C. The TGA curve is shown in FIG. 53 and indicates that the phase loses mass starting at about 115° C. The DVS curve is shown in FIG. 54 and the data indicate that the form absorbs less than about 0.35% of water up to 90% RH at 25° C. The material was found to not have changed form post experiment.

### Example 22. Compound of Formula I, Trans-Ferulic Co-Crystal, Form II

Compound of Formula I, trans-ferulic co-crystal, Form II was isolated when about 100 g of compound of Formula I, Form I was combined with about 1.1 equiv. (66 g) ferulic acid in a reactor. About 3000 mL of isopropyl acetate was added. The reactor temperature was increased to about 80° C. and a solution was observed. The reactor was cooled to 45° C. over about 1 hour. Seed was charged. Temperature was cycled from about 45° C. to about 20° C. six times. An aliquot was suction filtered and XRPD was taken and found to me a mixture of forms. Temperature was again cycled from about 45° C. to about 20° C. twelve times. About 82 g of solids were isolated. The reactor was rinsed with the liquors to recover the remaining solids. The isopropyl acetate was distilled portion wise down to about 1000 mL. The slurry was heat cycled from about 45° C. to about 20° C. four times. Slurry stirred for about 24 hours. The slurry was heat cycled from about 45° C. to about 20° C. six times. The slurry stirred for about 24 hours at about 45° C. The slurry was heated to about 80° C. About 15.7 g of compound of Formula I, Form I was added to bring the components back to a 1:1.5 Formula I:trans ferulic acid ratio. A solution was observed. Reactor temperature was set to about 45° C. Seed was charged. Immobile slurry formed. About 150 mL isopropyl acetate was added. Slurry stirred for about 3 hours at 45° C. The slurry was heat cycled from about 45° C. to about 20° C. six times. Solids were suction filtered and characterized by XRPD.

The Compound of Formula I, trans-ferulic co-crystal, Form II XRPD pattern is shown in FIG. 55 and is characterized by Tier 1 reflections at 4.7, 5.9, 25.9° 2θ, but also Tier 2 at 15.7, 18.9, 24.4° 2θ, and Tier 3 at 4.3, 9.3, 14.4° 2θ. A list of 2-theta peaks is provided below:

Peak Table

| Pos. [°2Th.] | Rel. Int. [%] |
|---|---|
| 4.3 | 20 |
| 4.7 | 100 |
| 5.9 | 63 |
| 9.3 | 19 |
| 11.7 | 17 |
| 14.4 | 21 |
| 15.7 | 45 |
| 16.7 | 8 |
| 18.9 | 22 |
| 19.5 | 15 |
| 21.3 | 6 |
| 23.5 | 16 |
| 24.4 | 37 |
| 25.9 | 60 |
| 27.4 | 6 |
| 28.9 | 13 |
| 30.0 | 11 |

The DSC curve is shown in FIG. 56 and displays an endothermic transition at about 136° C., 153° C., and 222° C. and an exothermic transition at about 290° C. The TGA curve is shown in FIG. 57 and indicates that the phase is unsolvated.

### Example 23. Compound of Formula I, Tromethamine Co-Crystal

Compound of Formula I, tromethamine co-crystal was isolated when about 8.8 g of compound of Formula I, Form I, about 2.6 g of tromethamine, and about 150 mL isopropanol was charged to a reactor and was agitated at 500 rpm. An additional about 55 mL isopropanol was charged to create a mobile slurry. The slurry stirred overnight. Solids were vacuum filtered and the cake was rinsed with isopropanol. The solids were dried in vacuum oven at 40° C. overnight.

The Compound of Formula I, tromethamine co-crystal XRPD pattern is shown in FIG. 58 and is characterized by Tier 1 reflections at 6.8, 20.4, 27.3° 2θ, but also Tier 2 at 16.4, 25.5, 17.3° 2θ, and Tier 3 at 21.6, 12.6, 23.7° 2θ. A list of 2-theta peaks is provided below:

Peak Table

| Pos. [°2Th.] | Rel. Int. [%] |
|---|---|
| 6.2 | 5 |
| 6.8 | 100 |
| 12.6 | 3 |
| 14.3 | 1 |
| 16.4 | 12 |
| 17.3 | 6 |
| 20.4 | 24 |
| 21.6 | 6 |
| 23.7 | 3 |
| 25.5 | 7 |
| 26.8 | 3 |
| 27.3 | 15 |
| 32.9 | 2 |
| 37.3 | 3 |

The DSC curve is shown in FIG. 59 and displays an endothermic transition at about 63° C. The TGA curve is shown in FIG. 60 and indicates that the phase loses mass in multiples stages starting at about 50° C. and another stage starting at about 140° C.

### Example 24. Compound of Formula I, L-Arginine Salt Form I

About 83 mg of the compound of Formula I was combined with about 59 mg L-arginine in a 4 mL vial with 1 mL acetone. A suspension was observed. About 1 mL THF was added. A sample was suction filtered and XRPD data was collected. Its XRPD pattern is shown in FIG. 48 and is characterized by Tier 1 reflections at 15.0, 23.3, 27.7° 2θ, but also Tier 2 at 19.4, 24.6, 29.9° 2θ, but also Tier 3 at 11.3, 16.7, 22.7° 2θ. A list of 2-theta peaks is provided below:

Peak Table

| Pos. [°2Th.] | Rel. Int. [%] |
|---|---|
| 11.3 | 8 |
| 15.0 | 54 |
| 16.0 | 7 |
| 16.7 | 12 |
| 17.4 | 8 |
| 18.3 | 4 |
| 19.4 | 35 |
| 20.8 | 8 |
| 22.7 | 13 |
| 23.3 | 100 |
| 24.6 | 15 |
| 27.7 | 58 |
| 28.7 | 14 |
| 29.9 | 15 |
| 32.7 | 11 |
| 33.6 | 4 |
| 36.1 | 6 |

### Example 25. Compound of Formula I, L-Arginine Salt Form II

About 439 mg of the compound of Formula I was combined with about 185 mg L-arginine and about 5 mL ethanol in a 20 mL vial with no stir bar. Sample was placed on nutating mixer overnight. A sample was suction filtered and XRPD data was collected. Its XRPD pattern is shown in FIG. 49 and is characterized by Tier 1 reflections at 6.8, 14.5, 20.5° 2θ, but also Tier 2 at 8.9, 11.8, 17.2° 2θ.

Peak Table

| Pos. [°2Th.] | Rel. Int. [%] |
|---|---|
| 6.8 | 100 |
| 8.9 | 3 |
| 10.5 | 2 |
| 11.8 | 4 |
| 12.9 | 3 |
| 14.5 | 6 |
| 17.2 | 7 |
| 17.9 | 3 |
| 19.2 | 2 |
| 20.5 | 11 |
| 22.1 | 4 |
| 24.4 | 2 |
| 25.5 | 2 |

### Example 26. Compound of Formula I, L-Arginine Salt Form III

The compound of Formula I, L-Arginine Salt Form II was dried in a vacuum oven at room temperature overnight. XRPD was collected from the dried solids. Its XRPD pattern is shown in FIG. 50 and is characterized by Tier 1 reflections at 7.3 and 9.6° 2θ. A list of 2-theta peaks is provided below:

Peak Table

| Pos. [°2Th.] | Rel. Int. [%] |
|---|---|
| 7.3 | 100 |
| 9.6 | 10 |

### Example 27. Compound of Formula I Tablets

Compound of Formula I tablets were manufactured using a dry granulation process. The compound of Formula I (free acid) was blended with microcrystalline cellulose, lactose monohydrate, croscarmellose sodium, and hydroxypropyl cellulose. The mixture was milled/dispersed, blended with intragranular magnesium stearate, dry-granulated by roller compaction and milled, and blended with extragranular magnesium stearate.

Example tablets were prepared having the following formulations.

### Example 27-1 (10 mg Formula I)

| Component | Wt. % | Mg/unit |
|---|---|---|
| Intragranular | | |
| Formula I | 7.410 | 10.00 |
| Lactose monohydrate | 41.05 | 55.41 |
| Microcrystalline cellulose | 41.05 | 55.41 |
| Croscarmellose sodium | 5.00 | 6.75 |
| Hydroxypropyl cellulose | 4.00 | 5.40 |
| Magnesium sterate | 0.750 | 1.02 |
| Extragranular | | |
| Magnesium Stearate | 0.750 | 1.02 |
| Total - Tablet Core | 100 | 135.0 |
| Opadry ® II Beige 85F570135 | 3.0 | 4.05 |

The total tablet weight was 139.05 mg.

### Example 27-2 (50 mg Formula I)

| Component | Wt. % | Mg/unit |
|---|---|---|
| Intragranular | | |
| Formula I | 30.12 | 50.00 |
| Lactose monohydrate | 29.69 | 49.29 |
| Microcrystalline cellulose | 29.69 | 49.29 |
| Croscarmellose sodium | 5.00 | 8.30 |
| Hydroxypropyl cellulose | 4.00 | 6.64 |
| Magnesium sterate | 0.750 | 1.25 |
| Extragranular | | |
| Magnesium Stearate | 0.750 | 1.25 |
| Total - Tablet Core | 100 | 166.0 |
| Opadry ® II Beige 85F570135 | 3.0 | 4.98 |

The total tablet weight was 170.98 mg.

### Example 27-3 (250 mg Formula I)

| Component | Wt. % | Mg/unit |
|---|---|---|
| Intragranular | | |
| Formula I | 30.120 | 250.00 |
| Lactose monohydrate | 29.690 | 246.43 |
| Microcrystalline cellulose | 29.690 | 246.43 |
| Croscarmellose sodium | 5.000 | 41.50 |
| Hydroxypropyl cellulose | 4.000 | 33.20 |
| Magnesium sterate | 0.750 | 6.23 |
| Extragranular | | |
| Magnesium Stearate | 0.750 | 6.23 |
| Total - Tablet Core | 100 | 830.0 |
| Opadry ® II Beige 85F570135 | 3.0 | 24.90 |

The total tablet weight was 854.90 mg.

### Example 27-4 (325 mg Formula I)

| Component | Wt. % | Mg/unit |
|---|---|---|
| Intragranular | | |
| Formula I | 30.12 | 325.0 |
| Lactose monohydrate | 29.69 | 320.4 |
| Microcrystalline cellulose | 29.69 | 320.4 |
| Croscarmellose sodium | 5.00 | 54.0 |
| Hydroxypropyl cellulose | 4.00 | 43.2 |
| Magnesium sterate | 0.750 | 8.1 |
| Extragranular | | |
| Magnesium Stearate | 0.750 | 8.1 |
| Total - Tablet Core | 100 | 1079 |
| Opadry ® II Beige 85F570135 | 3.0 | 32.4 |

The total tablet weight was about 1111 mg.

### Example 27-5 (450 mg Formula I)

| Component | Wt. % | Mg/unit |
|---|---|---|
| Intragranular | | |
| Formula I | 30.12 | 450.0 |
| Lactose monohydrate | 29.69 | 443.6 |
| Microcrystalline cellulose | 29.69 | 443.6 |
| Croscarmellose sodium | 5.00 | 74.7 |
| Hydroxypropyl cellulose | 4.00 | 59.8 |
| Magnesium sterate | 0.750 | 11.2 |
| Extragranular | | |
| Magnesium Stearate | 0.750 | 11.2 |
| Total - Tablet Core | 100 | 1494 |
| Opadry ® II Beige 85F570135 | 3.0 | 44.8 |

The total tablet weight was about 1539 mg.

For tablet Example 27-1 (7.4% w/w compound of Formula I), the final powder blend was compressed into tablet cores with a target weight of 135 mg to produce 10 mg strength tablets. For tablet Examples 27-2 and 27-3 (30.12% w/w compound of Formula I), the final powder blend was compressed into tablet cores with target weights of 166 mg and 830 mg to produce 50 mg and 250 mg strength tablets, respectively. For tablet Examples 27-4 and 27-5 (30.12% w/w compound of Formula I), the final powder blend was compressed into tablet cores with target weights of 1079 mg and 1494 mg to produce 325 mg and 450 mg strength tablets, respectively.

The tablet cores were film-coated with Opadry II Beige 85F570135 to a target weight gain of 3%. Purified water was used to prepare the film-coating suspension and was removed during the film-coating process to prepare the film coat.

### Example 28. Spray-Dried Dispersions

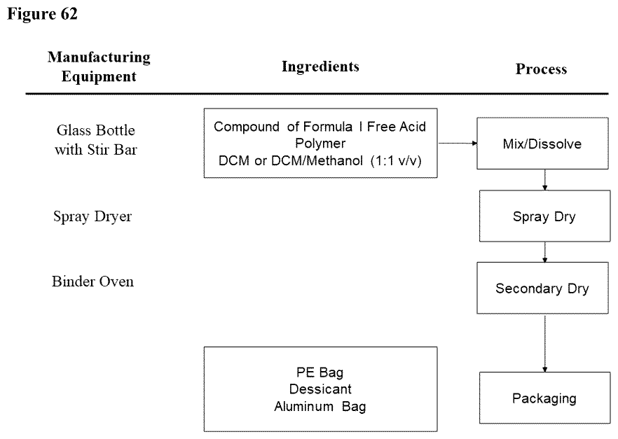

An example procedure for preparing a spray-dried dispersion of the compound of Formula I is depicted in the flow diagram of FIG. 62. A spray-dried dispersion (SDD) of the compound of Formula I was prepared using crystalline compound of Formula I free acid. The compound of Formula I and polymer were dissolved in dichloromethane (DCM, 100%) or DCM/methanol (1:1 v/v) to produce a feed solution containing 3.5-7% w/w solids. The feed solution was spray-dried using a Buchi Spray dryer and then further secondary-dried in a Binder oven set at 40-47° C. to yield the compound of Formula I SDD.

A similar process was used to prepare tablets as in Example 27, replacing the compound of Formula I with the SDD.

Exemplary SDDs prepared are shown in Table 3 below.

TABLE 3
Exemplary SDDs of the compound of Formula I

| | | | | | | PSD | | | |
|---|---|---|---|---|---|---|---|---|---|
| | | | | | Cpd. | (D₉₀, | | | |
| | | | | | Formula I | D₅₀, | T_{g} | | % Weight gain |
| | T_{g} Polymer (° C.) | Solids | Flow Rate | | to polymer | D₁₀ | SDD | Wt. % | at 25° C./80% |
| Polymer | (literature/measured) | (wt. %) | (mL/min.) | Solventª | ratio | (μm)) | (° C.) | volatiles^{b} | RH |
| None | n/a | 7 | 4 | DCM | n/a | NT | 84 | 0.5 | 1 |
| Copovidone | 106/111 | 7 | 16 | DCM | 1:1 | 10, 4, 1 | 97 | 1.8 | 10 |
| | | | | | 2:1 | 11, 4, 1 | 94 | 1.1 | 6 |
| HPMC-AS | 130/24 | 7 | 4 | DCM: | 1:1 | 16, 8, 2 | 89 | 0.7 | 3 |
| | | | | MeOH | 2:1 | 10, 5, 2 | 87 | 0.5 | NT |
| | | | | (1:1 v/v) | | | | | |
| Povidone | 171/165 | 7 | 4 | DCM | 1:1 | 6, 3, 1 | 118 | 2.1 | 21 |
| Eudragit L | —/—c | 3.5 | 4 | DCM: | 1:1 | 27, 14, | 152 | 1.6 | 4 |
| 100 | | | | MeOH | | 3 | | | |
| | | | | (1:1 v/v) | | | | | |
| Eudragit L | 110/125 | 3.5 | 4 | DCM: | 1:1 | 27, 12, | 110 | 0.9 | 3 |
| 100-55 | | | | MeOH | | 1 | | | |
| | | | | (1:1 v/v) | | | | | |

NT: Not tested
^{a}SDD was dried at ~40-50° C. overnight to remove remaining solvent.
^{b}At 100° C. by TGA.
^{c}Literature Tg past decomposition, could not identify by DSC.
All SDD samples were amorphous by XPRD and stayed amorphous after DVS runs

### Example 29. Antiviral Potency of Compound of Formula I Against a Panel of HIV-1 Mutants

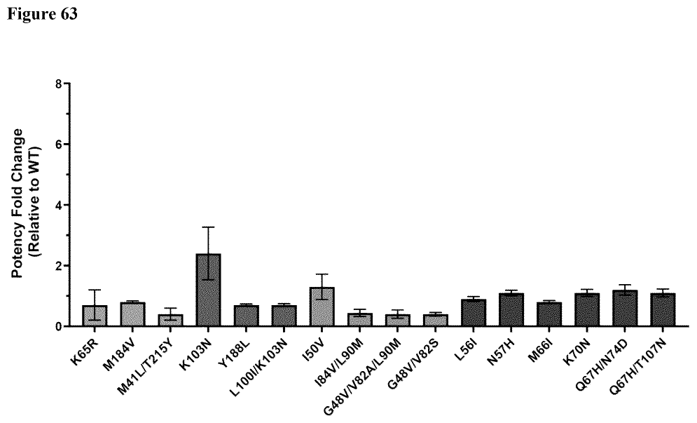

As shown in FIG. 63, the compound of Formula I showed antiviral potency against a panel of HIV-1 mutants resistant to other drug classes, including NRTI-resistant (NRTI-r), NNRTI-resistant (NNRTI-r), PI-resistant (PI-r), and CAI-resistant (CAI-r) mutants.

### Cells and Viruses:

The human T-lymphoblastoid MT-2 and SupTI cell lines were obtained from the NIH AIDS Reagent Program (Germantown, MD) and maintained in RPMI-1640 medium supplemented with 10% heat-inactivated fetal bovine serum, 100 units/mL penicillin, and 10 μg/mL streptomycin (complete RPMI media). Cells were passaged twice per week and kept at densities less than 800,000 cells per mL. Adherent HEK293T cells were obtained from the Gladstone Institute for Virology and Immunology (San Francisco, CA) and maintained at densities below 80% confluency in Dulbecco's Modified Eagle Medium (DMEM) (Gibco) supplemented with 10% heat-inactivated FBS, 100 units/mL penicillin, and 10 μg/mL streptomycin (complete DMEM media). Unless otherwise noted, HIV-1 recombinant strains carrying mutations were prepared by transfecting infectious proviral pLAI-based or pHXB2-based cDNA clones into MT-2 cells and harvesting the cell supernatants. The NNRTI-resistant HXB2-based viruses encoding reverse transcriptase (RT) mutations (K103N, Y181C, Y188L, L100I+K103N, and G190A) were constructed by site-directed mutagenesis. The NRTI-resistant LAI-based viruses encoding RT mutations K65R, M184V, and M41L+T215Y were constructed by site-directed mutagenesis. The PI-resistant HXB2-based viruses encoding mutations in the HIV-1 protease (PR) coding sequence (150V, 184V+L90M, G48V+V82A+L90M, and G48V+V82S) were produced in electroporated SupTI cells via homologous recombination {Cihlar 2003}. The single cycle wild-type (WT) and CAI-resistant reporter viruses encoding capsid (CA) mutations (L56I, N57H, M66I, K70N, Q67H+N74D, and Q67H+T107N) were produced by transient co-transfection of subconfluent HEK293T cells with the NL4.3-based env-deleted plasmid pKS13 encoding firefly luciferase {Balakrishnan 2013} (plus WT or capsid mutations) together with the pseudotyping expression vector pHCMV-G {Yee 1994} encoding the vesicular stomatitis virus G (VSV-G) envelope protein and then harvesting the cell supernatants 3 days later.

Table 4 shows the EC50 fold change for the compound of Formula I compared to control compounds when tested against a panel of HIV-1 mutants. The EC50 fold change range is the mean±standard deviation from at least three experiments run in triplicate. Controls: NRTIs (TAF, FTC), NNRTI (EFV), PIs (DRV, ATV), CAI (LEN).

TABLE 4
EC₅₀ Fold Change for Compound of Formula I
compared to Controls in HIV-1 Mutants Panel

| | EC₅₀ Fold Change, Range | | | |
|---|---|---|---|---|
| | NRTI-r | NNRTI-r | PI-r | CAI-r |
| Compound of Formula I | 0.4-0.8 | 0.7-2.4 | 0.4-1.3 | 0.8-1.2 |
| Controls | 2.7->131 | 66->82 | 5.3-31 | 20->350 |

### Example 30. Antiviral Potency of Compound of Formula I Against a INSTI Resistant Site-Directed HIV-1 Mutants

The compound of Formula I, along with control compounds bictegravir and raltegravir, were tested in a high-throughput 384-well assay format for ability to inhibit the replication of HIV-1 (HXB2) in MT-4 cells. Compounds were serially diluted (1:3) in 10 ul DMSO on 384-well plates and then diluted 187-fold into complete RPMI media using the uFLow and Biomek FX. 0.4 uL of compound was added in quadruplicate to 384-well assay plates. Each plate contained 8 test compounds, plus DMSO negative and 5 uM AZT positive controls. 1 mL aliquots of 2M MT-4 cells were pre-infected for 1 hr, @ 37° C. with 25 uL (MT-4) of either RPMI (mock-infected) or a fresh 1:250 dilution of an HIV-1 (HXB2 WT or site directed mutants containing either single INSTI Mutations E92Q, Y143R, Q148R, N155H, or R263K or double mutations E92Q/N155H, E138K/Q148K, or G140S/Q148R) concentrated ABI stock. Infected and uninfected cells were diluted in RPMI and 35 uL containing 2000 MT-4 cells was added to each well of assay plates, using the uFLow and subsequently incubated for 5 days at 37° C. in an incubator set at 5% CO2 and 90% humidity. After 5 days, 22 uL Cell Titer Glo (Promega, Madison, WI, Cat #G7573) was added to the assay plates with a Biotek uFlow Workstation. Plates were subsequently placed on a Perkin Elmer Envision Plate Reader for 5 minutes before the luminescence signal was read. EC50 values were defined as the compound concentration that causes a 50 decrease in luminescence signal, a measure of HIV-1 replication, and were calculated in Accord (online tool) using a one-site dose-response model to generate sigmoidal curve fits. Fold shifts for mutant viruses were obtained by calculating EC50 values relative to the HXB2 WT control.

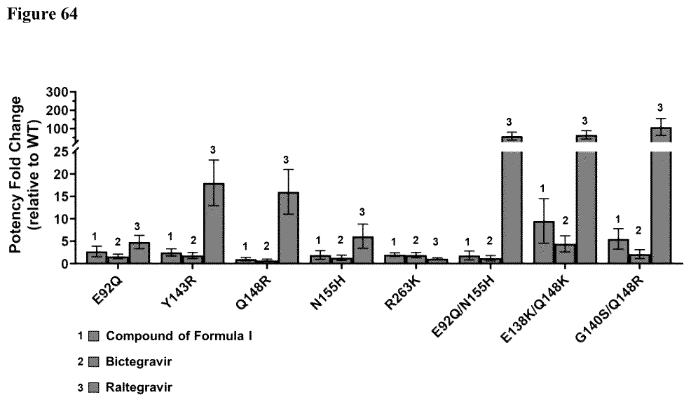

The results are shown in FIG. 64. The potency fold change (relative to WT) in FIG. 64 shows the mean value±standard deviation.

Table 5 shows the mean EC50 Fold Change for the compound of Formula I, bictegravir, and raltegravir for the INSTI resistant mutants tested. The mean fold change values (relative to WT) are from at least 3 independent experiments assayed in quadruplicate.

TABLE 5
Mean EC₅₀ Fold Change for the Compound of Formula I, bictegravir, and
raltegravir for INSTI resistant HIV-1 mutants

| | Mean EC₅₀ Fold Change (relative to WT) | | | | | | | |
|---|---|---|---|---|---|---|---|---|
| | | | | | | E92Q/ | E138K/ | G140S/ |
| | E92Q | Y143R | Q148R | N155H | R263K | N155H | Q148K | Q148R |
| Cpd. | 2.7 | 2.5 | 1.0 | 1.9 | 2.0 | 1.8 | 9.5 | 5.5 |
| Formula I | | | | | | | | |
| Bictegravir | 1.6 | 1.8 | 0.7 | 1.3 | 1.9 | 1.2 | 4.4 | 2.1 |
| Raltegravir | 4.8 | 18 | 16 | 6.1 | 1.1 | 58 | 6.5 | 108 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A solid oral dosage form comprising a compound of Formula I: 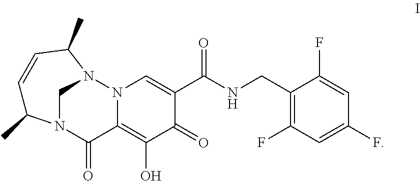
or a pharmaceutically acceptable salt thereof, and one or more excipients; wherein the compound of Formula I, or pharmaceutically acceptable salt thereof, is present in an amount of about 5 mg to about 1400 mg, wherein the amount is based on the free acid form.

2-18. (canceled)

19. The solid oral dosage form of claim 1, wherein the compound of Formula I, or pharmaceutically acceptable salt thereof, is present in an amount of about 1% to about 50% of the total weight of the dosage form.

20-27. (canceled)

28. The solid oral dosage form of claim 1, wherein the dosage form is a tablet.

29. (canceled)

30. (canceled)

31. The solid oral dosage form of claim 1, wherein the one or more excipients comprise a filler, a disintegrant, a lubricant, a surfactant, a pH modifier, a binder, or combinations thereof.

32-83. (canceled)

84. A solid oral dosage form comprising a compound of Formula I: 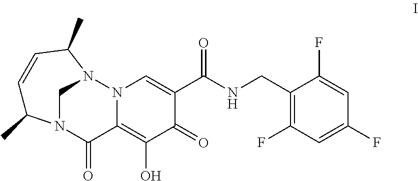
or a pharmaceutically acceptable salt thereof, in an amount of about 5 mg to about 1400 mg, wherein the amount is based on the free acid form;
a filler;
a disintegrant;
a binder; and
a lubricant.

85. The solid oral dosage form of claim 84, comprising:
about 5 wt. % to about 35 wt. % of the compound of Formula I, or a pharmaceutically acceptable salt thereof;
about 50 wt. % to about 90 wt. % of the filler;
about 1 wt. % to about 10 wt. % of the disintegrant;
about 1 wt. % to about 10 wt. % of the binder; and
about 0.1 wt. % to about 5 wt. % of the lubricant.

86. The solid oral dosage form of claim 1, comprising:
about 5 wt. % to about 35 wt. % of the compound of Formula I, or a pharmaceutically acceptable salt thereof;
about 20 wt. % to about 50 wt. % lactose;
about 20 wt. % to about 50 wt. % microcrystalline cellulose;
about 1 wt. % to about 10 wt. % croscarmellose sodium;
about 1 wt. % to about 10 wt. % hydroxypropyl cellulose; and
about 0.1 wt. % to about 2 wt. % magnesium stearate.

87. The solid oral dosage form of claim 86, comprising:
about 5 wt. % to about 35 wt. % of the compound of Formula I;
about 25 wt. % to about 45 wt. % lactose;
about 25 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

88. The solid oral dosage form of claim 86, comprising:
about 5 wt. % to about 10 wt. % of the compound of Formula I;
about 35 wt. % to about 45 wt. % lactose;
about 35 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

89. The solid oral dosage form of claim 86, comprising:
about 7.4 wt. % of the compound of Formula I;
about 41 wt. % lactose;
about 41 wt. % microcrystalline cellulose;
about 5 wt. % croscarmellose sodium;
about 4 wt. % hydroxypropyl cellulose; and
about 1.5 wt. % magnesium stearate.

90. The solid oral dosage form of claim 86, comprising:
about 28 wt. % to about 32 wt. % of the compound of Formula I;
about 25 wt. % to about 35 wt. % lactose;
about 25 wt. % to about 35 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

91. The solid oral dosage form of claim 86, comprising:
about 30 wt. % of the compound of Formula I;
about 30 wt. % lactose;
about 30 wt. % microcrystalline cellulose;
about 5 wt. % croscarmellose sodium;
about 4 wt. % hydroxypropyl cellulose; and
about 1.5 wt. % magnesium stearate.

92. The solid oral dosage form of claim 84, comprising:
the compound of Formula I in an amount of about 5 mg to about 500 mg;
the filler in an amount of about 60 mg to about 1000 mg;
the disintegrant in an amount of about 1 mg to about 100 mg;
the binder in an amount of about 1 mg to about 80 mg; and
the lubricant in an amount of about 0.5 mg to about 30 mg.

93. The solid oral dosage form of claim 86, comprising:
the compound of Formula I in an amount of about 5 mg to about 500 mg;
lactose in an amount of about 30 mg to about 500 mg;
microcrystalline cellulose in an amount of about 30 mg to about 500 mg;
croscarmellose sodium in an amount of about 1 mg to about 100 mg;
hydroxypropyl cellulose in an amount of about 1 mg to about 80 mg; and
magnesium stearate in an amount of about 0.5 mg to about 30 mg.

94. The solid oral dosage form of claim 84, comprising:
the compound of Formula I in an amount of about 10 mg to about 450 mg;
the filler in an amount of about 90 mg to about 900 mg;
the disintegrant in an amount of about 5 mg to about 80 mg;
the binder in an amount of about 5 mg to about 60 mg; and
the lubricant in an amount of about 0.5 mg to about 25 mg.

95. The solid oral dosage form of claim 86, comprising:
the compound of Formula I in an amount of about 10 mg to about 450 mg;
lactose in an amount of about 45 mg to about 450 mg;
microcrystalline cellulose in an amount of about 45 mg to about 450 mg;
croscarmellose sodium in an amount of about 5 mg to about 80 mg;
hydroxypropyl cellulose in an amount of about 5 mg to about 60 mg; and
magnesium stearate in an amount of about 0.5 mg to about 25 mg.

96. The solid oral dosage form of claim 84, comprising:
the compound of Formula I in an amount of about 10 mg to about 250 mg;
the filler in an amount of about 90 mg to about 500 mg;
the disintegrant in an amount of about 5 mg to about 45 mg;
the binder in an amount of about 5 mg to about 35 mg; and
the lubricant in an amount of about 0.5 mg to about 20 mg.

97. The solid oral dosage form of claim 86, comprising:
the compound of Formula I in an amount of about 10 mg to about 250 mg;
lactose in an amount of about 45 mg to about 250 mg;
microcrystalline cellulose in an amount of about 45 mg to about 250 mg;
croscarmellose sodium in an amount of about 5 mg to about 45 mg;
hydroxypropyl cellulose in an amount of about 5 mg to about 35 mg; and
magnesium stearate in an amount of about 0.5 mg to about 20 mg.

98. The solid oral dosage form of claim 97, comprising:
the compound of Formula I in an amount of about 10 mg;
lactose in an amount of about 55 mg;
microcrystalline cellulose in an amount of about 55 mg;
croscarmellose sodium in an amount of about 7 mg;
hydroxypropyl cellulose in an amount of about 5 mg; and
magnesium stearate in an amount of about 2 mg.

99. The solid oral dosage form of claim 97, comprising:
the compound of Formula I in an amount of about 50 mg;
lactose in an amount of about 49 mg;
microcrystalline cellulose in an amount of about 49 mg;
croscarmellose sodium in an amount of about 8 mg;
hydroxypropyl cellulose in an amount of about 7 mg; and
magnesium stearate in an amount of about 2.5 mg.

100. The solid oral dosage form of claim 97, comprising:
the compound of Formula I in an amount of about 250 mg;
lactose in an amount of about 246 mg;
microcrystalline cellulose in an amount of about 246 mg;
croscarmellose sodium in an amount of about 42 mg;
hydroxypropyl cellulose in an amount of about 33 mg; and
magnesium stearate in an amount of about 12 mg.

101. The solid oral dosage form of claim 95, comprising:
the compound of Formula I in an amount of about 325 mg;
lactose in an amount of about 320 mg;
microcrystalline cellulose in an amount of about 320 mg;
croscarmellose sodium in an amount of about 54 mg;
hydroxypropyl cellulose in an amount of about 43 mg; and
magnesium stearate in an amount of about 16 mg.

102. The solid oral dosage form of claim 95, comprising:
the compound of Formula I in an amount of about 450 mg;
lactose in an amount of about 444 mg;
microcrystalline cellulose in an amount of about 444 mg;
croscarmellose sodium in an amount of about 75 mg;
hydroxypropyl cellulose in an amount of about 60 mg; and
magnesium stearate in an amount of about 22 mg.

103. The solid oral dosage form of claim 84, wherein the dosage form is a tablet comprising a tablet core and a film coat; and wherein the tablet core comprises:
the compound of Formula I or pharmaceutically acceptable salt thereof;
the filler;
the disintegrant;
the binder; and
the lubricant.

104-133. (canceled)

134. A pharmaceutical composition comprising:
about 5 wt. % to about 35 wt. % of a compound of Formula I: 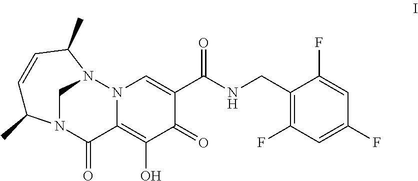
or a pharmaceutically acceptable salt thereof;
about 50 wt. % to about 90 wt. % of a filler;
about 1 wt. % to about 10 wt. % of a disintegrant;
about 1 wt. % to about 10 wt. % of a binder; and
about 0.1 wt. % to about 5 wt. % of a lubricant.

135. A pharmaceutical composition, comprising:
about 5 wt. % to about 35 wt. % of a compound of Formula I: 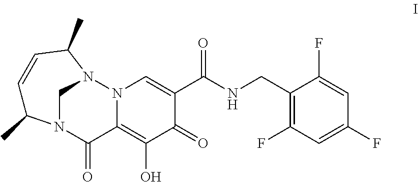
or a pharmaceutically acceptable salt thereof;
about 20 wt. % to about 50 wt. % lactose;
about 20 wt. % to about 50 wt. % microcrystalline cellulose;
about 1 wt. % to about 10 wt. % croscarmellose sodium;
about 1 wt. % to about 10 wt. % hydroxypropyl cellulose; and
about 0.1 wt. % to about 2 wt. % magnesium stearate.

136. The pharmaceutical composition of claim 135, comprising:
about 5 wt. % to about 35 wt. % of the compound of Formula I;
about 25 wt. % to about 45 wt. % lactose;
about 25 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

137. The pharmaceutical composition of claim 135, comprising:
about 5 wt. % to about 10 wt. % of the compound of Formula I;
about 35 wt. % to about 45 wt. % lactose;
about 35 wt. % to about 45 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

138. The pharmaceutical composition of claim 135, comprising:
about 7.4 wt. % of the compound of Formula I;
about 41 wt. % lactose;
about 41 wt. % microcrystalline cellulose;
about 5 wt. % croscarmellose sodium;
about 4 wt. % hydroxypropyl cellulose; and
about 1.5 wt. % magnesium stearate.

139. The pharmaceutical composition of claim 135, comprising:
about 28 wt. % to about 32 wt. % of the compound of Formula I;
about 25 wt. % to about 35 wt. % lactose;
about 25 wt. % to about 35 wt. % microcrystalline cellulose;
about 3 wt. % to about 7 wt. % croscarmellose sodium;
about 2 wt. % to about 6 wt. % hydroxypropyl cellulose; and
about 0.1 wt. % to about 2 wt. % magnesium stearate.

140. The pharmaceutical composition of claim 135, comprising:
about 30 wt. % of the compound of Formula I;
about 30 wt. % lactose;
about 30 wt. % microcrystalline cellulose;
about 5 wt. % croscarmellose sodium;
about 4 wt. % hydroxypropyl cellulose; and
about 1.5 wt. % magnesium stearate.

141-143. (canceled)

144. A method of treating or preventing HIV infection in a human, comprising orally administering to the human the solid oral dosage form of claim 1.

145-167. (canceled)

168. A method of treating or preventing HIV infection in a human, comprising orally administering to the human the solid oral dosage form of claim 84.
